(19) United States
(12) Patent Application Publication
(10) Pub. No.: US 2025/0346938 A1
(43) Pub. Date: Nov. 13, 2025

(54) ATP-DEPENDENT C-TERMINAL MODIFICATION OF POLYPEPTIDES

(71) Applicant: Wisconsin Alumni Research Foundation, Madison, WI (US)

(72) Inventors: Amy Weeks, Madison, WI (US); Clara Frazier, Madison, WI (US); Debashrito Deb, Madison, WI (US)

(73) Assignee: Wisconsin Alumni Research Foundation, Madison, WI (US)

(21) Appl. No.: 19/203,611

(22) Filed: May 9, 2025

Related U.S. Application Data
(60) Provisional application No. 63/646,193, filed on May 13, 2024.

(51) Int. Cl. C12P 21/02; C12N 9/54; C12N 9/88

(52) U.S. Cl. CPC C12P 21/02; C12N 9/54; C12N 9/88; C12Y 304/21062; C12Y 404/01001; C07K 2319/00

(57) ABSTRACT

A polypeptide fusion comprising a polypeptide having a C-terminus and a Thioesterification C-terminal Handle (TeCH-tag) fused to the C-terminus of the polypeptide, and a method of modifying the C-terminus of a polypeptide using the polypeptide fusion. The TeCH-tag comprises a sequence of formula (X),X′, wherein X is any amino acid, n is an integer from 6 to 55, and X′ is an amino acid other than asparagine. The TeCH-tag is a substrate of an E1-like superfamily enzyme, and the method comprises reacting the polypeptide fusion, the E1-like superfamily enzyme, and ATP under conditions to O-AM Pylate the C-terminus of the polypeptide fusion; and reacting the C-terminally O-AM Pylated polypeptide fusion with a nucleophile comprising a functional group to provide a modified polypeptide fusion comprising the C-terminal functional group.

### CROSS REFERENCE TO RELATED APPLICATIONS

Priority is hereby claimed to provisional application Ser. No. 63/646,193, filed May 13, 2024, which is incorporated herein by reference.

### FEDERAL FUNDING STATEMENT

This invention was made with government support under GM 149548 awarded by the National Institutes of Health. The government has certain rights in the invention.

### SEQUENCE LISTING

The instant application contains a Sequence Listing which has been submitted in an XML file with the USPTO through Patent Center and is hereby incorporated by reference in its entirety. The Sequence Listing XML, created on Mar. 3, 2025, is named “SEQ_LIST--09824603-P240096U S02.xml” and is 100,611 bytes in size.

### FIELD OF THE DISCLOSURE

The present disclosure is related to a method of modifying polypeptides, specifically methods of modifying the C-termini of polypeptides including the addition of C-terminal thioesters.

### BACKGROUND

Protein-bound C-terminal thioesters are electrophilic groups that function in enzymatic catalysis, serve as intermediates in protein splicing, and enable installing post-translational modifications including ubiquitin and ubiquitin-like proteins. Although nature has evolved several strategies to generate C-terminal thioesters, only one strategy, intein-mediated protein splicing, has been harnessed as a tool for producing recombinant protein C-terminal thioesters. Thioester-modified proteins serve as versatile intermediates for synthesis of chemically tailored proteins, and thus are desirable. Recombinant C-terminal thioesters can be deployed for native chemical ligation to N-terminal Cys peptides in a method termed expressed protein ligation (EPL). Recombinant C-terminal thioesters can also be used as substrates for the engineered peptide ligase subtiligase, which catalyzes Cys-independent ligation to synthetic peptides of arbitrary sequence in a process known as enzyme-catalyzed EPL. However, subtiligase-catalyzed thioester hydrolysis competes with ligation and results in a C-terminal carboxylate that is not a substrate for subtiligase. Moreover, the thioester substrate cannot be regenerated because intein-mediated C-terminal thioester generation is not a multi-turnover process. Therefore, although engineered inteins have enabled precise manipulation of protein structure to advance the understanding of a broad range of biological questions, a method for enzyme-catalyzed protein C-terminal thioester synthesis would improve the broad applicability of protein ligation for precision tailoring of protein chemical structure.

In living systems, ATP provides an energetic driving force for peptide bond synthesis. For example, activation of the α-carboxylate of amino acids or protein/peptide C termini by adenylation enables formation of ester and thioester intermediates that function in diverse pathways including ribosomal protein synthesis, non-ribosomal peptide synthesis, and the ubiquitination cascade. Cleavage of ATP to AMP and pyrophosphate (PPi) provides a large thermodynamic driving force (ΔG°′=−45.6 kj/mol for hydrolysis) for otherwise unfavorable biosynthetic reactions. In both protein translation and non-ribosomal peptide synthesis, amino acids are initially activated by ATP-dependent adenylation to hydrolytically unstable aminoacyl-AM Ps, which are converted to aminoacyl-tRNAs or aminoacyl-peptidyl carrier proteins that can be used in protein synthesis or non-ribosomal peptide synthesis, respectively. In contrast, enzyme-generated adenylates have not been used as activated intermediates for in vitro protein bioconjugation by bioorganic chemists. Instead, the most widely adopted protein bioconjugation methods rely on reversible enzyme-catalyzed transpeptidation reactions or non-enzymatic pre-synthesis of activated precursors to provide a driving force for peptide bond formation.

The E1-like (ThiF) enzyme superfamily is comprised of structurally and mechanistically related proteins that share a common mechanistic step involving adenylation of a C-terminal α-carboxylate to generate a reactive peptidyl-O-AMP mixed anhydride electrophile. Based on its capacity to react with diverse nucleophiles, this shared intermediate provides biological systems with access to protein and peptide C-terminal thioesters, thiocarboxylates, succinimides and (iso) peptide bonds that function in biological processes ranging from ubiquitination to biosynthesis of metabolites including thiamin, molybdopterin, and ribosomally synthesized and post-translationally modified peptide (RiPP) natural products. Despite their versatility, E1-like enzymes have not been developed for in vitro applications in peptide bond synthesis as many of the best studied E1-like enzymes require ubiquitin-like proteins as their substrates. In contrast to many other E1-like enzymes, E. coli M ccB, which natively functions in formation of a C-terminal phosphoramidate linkage in biosynthesis of the RiPP natural product microcin C7, accepts a short heptapeptide as its substrate. This feature makes M ccB a promising candidate tool for protein bioconjugation. However, MccB's native reaction chemistry does not couple C-terminal adenylation to a peptide bond formation reaction that would be useful for protein bioconjugation.

What is needed are alternative methods of producing C-terminal protein modifications including thioester modification.

### SUMMARY

In biological systems, ATP provides an energetic driving force for peptide bond formation, but protein chemists lack tools that emulate this strategy. The present disclosure developed an ATP-driven platform for C-terminal activation and peptide ligation based on M ccB, a bacterial ancestor of ubiquitin-activating (E1) enzymes. It is shown that M ccB can act on non-native substrates to generate an O-A M Pylated electrophile that reacts with exogenous nucleophiles to form diverse C-terminal functional groups including thioesters, a versatile class of biological intermediates that have been exploited for protein C-terminal bioconjugation. By mining the natural diversity of the M ccB family, the present disclosure identifies both epitope-specific and more promiscuous M ccBs. It is shown that epitope-specific M ccB activity can be directed toward specific proteins of interest to enable high-yield, ATP-driven protein bioconjugation, while promiscuous MccB activity can be deployed for synthesis of peptide thioester substrates for bioconjugation. The method disclosed herein mimics the chemical logic of biological peptide bond synthesis for high-yield in vitro manipulation of protein structure with molecular precision.

Specifically, disclosed and claimed herein is a polypeptide fusion comprising a polypeptide having a C-terminus and a peptide tag fused to the C-terminus of the polypeptide, wherein the peptide tag comprises a sequence of formula (X),X′, wherein X is any amino acid, n is an integer from 6 to 55, and X′ is an amino acid other than asparagine, and wherein the polypeptide is not fused to the peptide tag in nature.

In certain versions, X′ is selected from alanine, glycine, serine, and threonine. The peptide tag may be derived from a peptide selected from SEQ ID NOs: 5-51, wherein the C-terminal asparagine of the peptide is replaced with X′. In certain embodiments, the peptide tag is selected from SEQ ID NOs: 52-55.

Also disclosed herein is a method of modifying a C-terminus of a polypeptide, comprising:
providing the polypeptide, wherein the polypeptide is a substrate of an E1-like superfamily enzyme;
reacting the polypeptide, the E1-like superfamily enzyme, and ATP under conditions to O-A M Pylate the C-terminus of the polypeptide; and
reacting the C-terminally O-A M Pylated polypeptide with a nucleophile comprising a functional group to provide a modified polypeptide comprising the C-terminal functional group.

The polypeptide may be any peptide, modified or unmodified, that comprises a C-terminal carboxylate. In certain versions, the polypeptide is the polypeptide fusion described above. Non-limiting examples of the nucleophile include a thiol, a hydrazine, an alkoxyamine, a hydroxylamine, an amine, and an alcohol.

In one version, the nucleophile is a thiol nucleophile and the C-terminal functional group is a thioester. Non-limiting examples of the thiol nucleophile include N-acetyl-L-cysteine, N-acetylcysteamine, sodium 2-mercaptoethane sulfonate (M esna), dithiothreitol (DTT), and L-cysteine (Cys).

The method may further comprise transthiolating the C-terminal thioester polypeptide. The C-terminal thioester polypeptide may be transthiolated with dithiothreitol (DTT), betamercaptoethanol (BM E), 4-mercaptophenylacetic acid (MPAA), or other thiols.

The method may further comprise reacting the C-terminal functional group with a bioconjugation agent. The C-terminal functional group may be a thioester, and the bioconjugation agent may comprise (but is not limited to) an azide, a malemide, a para-fluoro compound, an alkene, an alkyne, or a vinyl sulfone. The bioconjugation agent may further comprise a cargo molecule. The method may further comprise reacting the bioconjugation agent with a reactive molecule comprising a cargo molecule to provide a fusion polypeptide labeled with the cargo molecule. Non-limiting examples of the cargo molecule include biotin, an imaging agent, a pharmaceutical agent, a nanoparticle, a radiolabel, a polymer, and an amino acid. In some embodiments, peptides modified with azide or alkyne groups, or with cysteine, are ligated to modify the C terminus with cargoes.

The method may further comprise exchanging the thioester with reactive moiety comprising an N-terminal cysteine to form a peptide bond.

The method may further comprise reacting the C-terminal thioester-modified polypeptide with a target peptide in the presence of a ligase to provide a target peptide fused to the C-terminus of the polypeptide.

In certain versions, the E1-like superfamily enzyme is selected from the group consisting of Escherichia coli M ccB (SEQ ID NO: 1), Helicobacter pylori M ccB (SEQ ID NO: 2), Lactobacillus johnsonii M ccB (SEQ ID NO: 3), and Histophilus somni M ccB (SEQ ID NO: 4), or a polypeptide with at least 90%, 91%, 92%, 93%, 94%, 95%, 96%, 97%, 98%, or 99% sequence identity to any of the foregoing enzymes.

The objects and advantages of the disclosure will appear more fully from the following detailed description of the preferred embodiment of the disclosure made in conjunction with the accompanying drawings.

### BRIEF DESCRIPTION OF THE DRAWINGS

FIGS. 1A-1F. M ccB catalyzes C-terminal O-A M Pylation of non-native substrates. (FIG. 1A) M ccB enzymes are members of the E1-like/ThiF superfamily, which have in common the formation of a C-terminal peptidyl-O-AMP intermediate. (FIG. 1B) Members of the E1-like superfamily that act on C-termini generally recognize ubiquitin-like proteins that terminate in Gly as their substrates; the M ccB family is an exception as it acts on short peptides (M ccA s) that terminate in Asn. (FIG. 1C) The reaction catalyzed by M ccB in its native context. (FIG. 1D) The C-terminal residue of E. coli M ccA was varied to the 19 non-native amino acids and MccB's ability to catalyze C-terminal O-A M Pylation was assayed using a colorimetric assay for pyrophosphate. (FIG. 1E) M ccA-N7A, N7G, N7S, and N 7T stimulate M ccB-catalyzed pyrophosphate formation (left column), while only wt M ccA supports formation of a stably A M Pylated product detectable by HPLC-MS (right column). (FIG. 1F) Steady-state kinetic parameters for M ccB-catalyzed pyrophosphate release when wt MccA or M ccA variants are used as substrates. Kinetic parameters are given as value+standard error.

FIGS. 2A-2E. M ccB catalyzes formation of a peptidyl-O-A M P intermediate that can react with exogenous nucleophiles. (FIG. 2A) MccA -O-AMP undergoes reaction with its C-terminal Asn side chain to form a succinimide intermediate and subsequently a stably N-AM Pylated product that can be detect by LC-MS (top). In contrast, M ccA-N 7G-O-AM P is expected to undergo hydrolysis on a timescale incompatible with LC-MS detection. (FIG. 2B) LC-MS analysis of M ccB reactions with wild-type M ccA (left) or M ccA-N 7G (right) as a substrate. (FIG. 2C) Hypothesized M ccA-N7G-O-AMP reactivity with exogenous nucleophiles. (FIG. 2D) M ccB-catalyzed hydrazine modification of M ccA-N 7G. ESI-MS spectra of unmodified M ccA-N7G (left) and M ccA-N 7G (250 μM) incubated with M ccB (5 μM), ATP (5 mM), and hydrazine (150 mM) reveal that M ccA-N7G-O-AMP can react with exogenous nucleophiles. (FIG. 2E) Radial heatmaps show the percent conversion of MccA to nucleophile-modified M ccA for a panel of alkoxyamine, hydrazine, amine, and thiol nucleophiles. Percent conversion was calculated using peak areas from reactant and product extracted ion chromatograms.

FIG. 3. HPLC-MS quantification of nucleophile ligation of M ccA-N 7G. (Panel A) Quantification of percent modified M ccA-N 7G generated by M ccB in presence of 150 mM nitrogen nucleophile, 250 μM peptide substrate, and 5 mM ATP after a 16 h reaction. (Panel B) Quantification of percent modified M ccA-N 7G generated by M ccB in presence of 150 mM thiol nucleophile, 250 μM peptide substrate, and 5 mM ATP after a 16 h reaction.

FIG. 4. M ccB-generated thioesters undergo transthioesterification, S-to-N acyl transfer, and native chemical ligation. (Panel A) MccA-N7G thioester can undergo transthioesterification with M PAA, a thiol nucleophile that cannot directly capture M cc-N7G-O-AMP. (Panel B) MccA-N7G-O-AMP can undergo thioesterification and S-to-N acyl shift with Cys to form a peptide bond. (Panel C) In the presence of ATP and Mesna, M ccB catalyzes native chemical ligation between an unactivated peptide and an N-terminal Cys peptide.

FIG. 5. Transthioesterification of M ccA-N7G Mesna thioester with dithiothreitol (DTT). (Panel A) Scheme of initial Mesna thioesterification of MccA N7G followed by transthiolation with competing DTT. (Panel B) Extracted ion chromatograms of the M ccA N 7G reactant, M esna thioester product, and DTT thioester product. The masses of both singly and doubly charged species are included. (Panel C) HPLC-MS quantification of DTT transthiolation of M ccA N7G thioester.

FIG. 6. Transthioesterification of MccA-N7G Mesna thioester with 4-mercaptophenylacetic acid (M PAA). (Panel A) Scheme of initial Mesna thioesterification of M ccA N7G followed by transthiolation with competing M PAA. (Panel B) Extracted ion chromatograms of the M ccA N 7G reactant, M esna thioester product, and M PAA thioester product. The masses of both singly and doubly charged species are included. (Panel C) HPLC-MS quantification of M PAA transthiolation of M ccA N7G thioester.

FIG. 7. Cysteine thioesterification of MccA-N 7G undergoes S-to-N acyl shift to form a new amide bond. (Panel A) Scheme of initial Cysteine thioesterification of M ccA-N 7G followed by S-to-N acyl shift to free the thiol side chain for maleimide ligation. (Panel B) Extracted ion chromatograms of the M ccA N7G reactant, Cys captured product, and Cys-maleimide product. The masses of both singly and doubly charged species are included. (Panel C) HPLC-MS quantification of Cys capture and maleimide modification.

FIG. 8. Native chemical ligation (NCL) application of M ccA-N 7G M esna thioester with CysTrp dipeptide. (Panel A) Scheme of initial M esna thioesterification of M ccA-N 7G followed by transthiolation with a CysTrp dipeptide. The thioester undergoes an S-to-N acyl shift to form a stable amide bond. (Panel B) Extracted ion chromatograms of the M ccA N 7G reactant, Mesna thioester, and CysTrp ligation product. The masses of both singly and doubly charged species are included. (Panel C) HPLC-MS quantification of M esna thioesterification and CysTrp ligation.

FIGS. 9A-9E. Fusion of the Thioesterification C-terminal Handle (TeCH-tag) to proteins enables M ccB-catalyzed, ATP-dependent formation of C-terminal thioesters. (FIG. 9A) Fusion of the TeCH-tag to GFP for C-terminal thioesterification. (FIG. 9B) M ccB catalyzes ATP-dependent thioesterification of GFP-TeCH-tag within 30 min. (FIG. 9C) M ccB catalyzes C-terminal thioesterification of TeCH-tag fusions of MBP, the catalytic domain of protein tyrosine phosphatase 1B (PT P1B 1-321), protein L, an α-GFP recombinant antibody, and an EGFR-targeting affibody. The * indicates an α-gluconylated form of protein L that is an artifact of His-tag purification. (FIG. 9D) M ccB catalyzes C-terminal thioesterification of GFP-TeCH-tag with cysteine, with subsequent S-to-N acyl shift leading to formation of a peptide bond as evidenced by the maleimide reactivity of the bioconjugate. (FIG. 9E) GFP-TeCH-tag can be modified by expressed protein ligation via a Mesna thioester intermediate in a one-pot reaction with M ccB, ATP, Mesna, and the peptide CGAGS-azidoalanine.

FIGS. 10A-10S. Deconvoluted mass spectrum from RP-HPLC-ESI-MS data. (FIG. 10A) GFP-TeCH. Calc: 27,947 Da. Observed: 27,947 Da. (FIG. 10B) M ccB-generated GFP-TeCH-Mes C-terminal thioester. Calc: 28,071 Da. Observed: 28,071 Da. (FIG. 10C) Intein-generated GFP-TeCH-Mes C-terminal thioester. Calc: 27,927 Da (M es thioester), 27,803 Da (hydrolyzed thioester). Observed: 27,927 Da (Mes thioester), 27,803 Da (hydrolyzed thioester). (FIG. 10D) Intein-generated GFP-TeCH-Mes C-terminal thioester treated with MccB, ATP, and Mesna. Calc: 27,927 Da (Mes thioester), 27,803 Da (hydrolyzed thioester). Observed: 27,927 Da (M es thioester). (FIG. 10E) MBP-TeCH. Calc: 43,599 Da. Observed: 43,603 Da. (FIG. 10F) M ccB-generated MBP-TeCH-M es C-terminal thioester. Calc: 43,723 Da. Observed: 43,724 Da. (FIG. 10G) PTP1B₁₋₃₂₁-TeCH. Calc: 38,373 Da. Observed: 38,375 Da. (FIG. 10H) M ccB-generated PTP1B₁₋₃₂₁-M es C-terminal thioester. Calc: 38,497 Da. Observed: 38,499 Da. (FIG. 10I) Anti-GFP recombinant antibody (aGFP rAb). Calc: 48,000 Da. Observed: 48,001 Da. (FIG. 10J) Anti-GFP recombinant antibody (aGFP rAb) with an M ccB-generated C-terminal M es thioester on the heavy chain. Calc: 48,124 Da. Observed: 48,125 Da. (FIG. 10K) Protein L-TeCH. Calc: 42,679 Da (unmodified), 42,852 Da (N-gluconylated). Observed: 42,682 Da, 42,857 Da. (FIG. 10L) M ccB-generated protein L-TeCH-Mes C-terminal thioester. Calc: 42,803 Da (unmodified), 42,976 Da (N-gluconylated). Observed: 42,804 Da, 42,989 Da. (FIG. 10M) ZEGFR-TeCH. Calc: 10,571 Da (M et excised). Observed: 10,571 Da. (FIG. 10N) M ccB-generated zEGFR-TeCH-M es C-terminal thioester. Calc: 10,695 Da. Observed: 10,695 Da. (FIG. 10O) M ccB-generated GFP—TeCH-Cys. Calc: 28,050 Da. Observed: 28,050 Da. (FIG. 10P) GFP—TeCH-Cys-biotin-maleimide. Calc: 28,576 Da. Observed: 28,576 Da. (FIG. 10Q) GFP—TeCH-Cys-Cy5-maleimide. Calc: 28,655 Da. Observed: 28,655 Da. (FIG. 10R) GFP—TeCH-CGAGSazA. Calc: 28,433 Da. Observed: 28,434 Da. (FIG. 10S) GFP—TeCH-CGAGSazA after reaction with dibenzocyclooctyne-biotin. Calc: 29,183 Da. Observed: 29,184 Da.

FIG. 11. Thioesterification of eGFP-TeCH using Mesna. (Panel A) Scheme of cysteine free eGFP-TeCH fusion thieosterification with Mesna. Reactions were performed with 5 μM MccB, 50 μM eGFP-TeCH fusion, 5 mM ATP, and the appropriate concentration of M esna/TCEP in 75 mM Tris, pH 8.0. Reactions were quenched at selected time points by double volume addition of 0.6% TFA. (Panel B) Representative deconvoluted traces of HPLC-MS intact protein analysis. (Panel C) HPLC-MS quantification of M esna thioesterification with 5 mM or 1 mM M esna/TCEP solutions.

FIG. 12. C-terminal ligation of eGFP-TeCH with Cys. (Panel A) Scheme of cysteine free eGFP-TeCH fusion ligation with C-terminal Cys. Reactions were performed with 5 μM MccB, 50 μM eGFP-TeCH fusion, 5 mM ATP, and the appropriate concentration of Cys/TCEP in 75 mM Tris, pH 8.0. Reactions were quenched at selected time points by double volume addition of 0.6% TFA. (Panel B) Representative deconvoluted traces of HPLC-MS intact protein analysis of eGFP-TeCH tag fusion reactions with 5 mM Cys/TCEP. (Panel C) HPLC-MS quantification of Cys ligation with 5 mM or 1 mM Cys/TCEP solutions.

FIG. 13. ESI-MS spectrum for CGAGS-3-azido-L-Ala C-terminal amide. The peptide was observed in its oxidized disulfide form.

FIGS. 14A-14F. Natural MccA/M ccB diversity encompasses orthogonal enzymes for C-terminal protein modification. (FIG. 14A) A positional scanning peptide library revealed that M ccB is an epitope-specific enzyme. (FIG. 14B) The M ccB enzyme family harbors numerous homologs that act on substrates distinct from E. coli M ccA. Enzymes from H. pylori, L. johnsonii, and H. somni are highlighted in cyan. The E. coli, H. pylori, and L. johnsonii M ccB were found to be mutually orthogonal enzymes. (FIG. 14C) The E. coli, H. pylori, and L. johnsonii enzymes catalyze C-terminal thioesterification of their respective M ccA-N 7G sequences and are orthogonal to one another. (FIG. 14D) LC-MS analysis of GFP-EcTeCH, GFP-HpTeCH, and GFP-LjTeCH shows that M ccA-N 7Gs from E. coli, H. pylori, and L. johnsonii can be deployed as TeCH-tags for C-terminal protein modification. (FIG. 14E) M ccBs from E. coli, H. pylori, and L. johnsonii modify only proteins tagged with their cognate TeCH-tags. (FIG. 14F) In a mixture of GFPs fused to TeCH-tags from E. coli, H. pylori, and L. johnsonii, the M ccB homologs from E. coli, H. pylori, and L. johnsonii selectively modify their cognate TeCH-tags.

FIG. 15. Quantification of conversion of M ccA positional scanning peptide library variants to N-AM Pylated product. Reactions contained 5 mM M ccB, 250 mM M ccA variant, and 5 mM ATP and were incubated for 16 h at room temperature. Percent conversion was calculated from product and reactant extracted ion chromatograms using the formula (product peak area)/(product peak area+reactant peak area)*100. (A) M 1X variants. (B) R 2X variants. (C) T3X variants. (D) G4X variants. (E) N5X variants. (F) A 6X variants. (G) N 7X variants.

FIG. 16. Steady-state kinetic analysis of MccA with alanine substitutions at positions 1-5 and 7 or a serine substitution at position 6. A ssays were performed using an enzyme-coupled pyrophosphate release assat at the M ccB concentration indicated on each plot. (A) Wild-type M ccA. (B) MccA-M 1A. (C) M ccA -R2A. (D) MccA-T3A. (E) M ccA-G4A. (F) M ccA-N5A. (G) MccA-A6S. (H) M ccA-N7A.

FIG. 17. C-terminal thioesterification activity of E. coli, H. pylori, and L. johnsonii M ccB homologs measured by LC-TOF MS. Reactions contained 5 mM M ccB, 250 mM MccA-N7G, 100 mM Mesna, and 5 mM ATP and were incubated for 16 h at room temperature.

FIGS. 18A-18D. Deconvoluted mass spectrum from RP-HPLC-ESI-MS data. (FIG. 18A) GFP-LjTeCH. Calc: 28,113 Da. Observed: 28,114 Da. (FIG. 18B) M ccB-generated GFP-LjTeCH-M es C-terminal thioester. Calc: 28,237 Da. Observed: 28,238 Da. (FIG. 18C) GFP-HpTeCH. Calc: 28,096 Da. Observed: 28,096 Da. (FIG. 18D) M ccB-generated GFP-HpTeCH-Mes C-terminal thioester. Calc: 28,220 Da. Observed: 28,219 Da.

FIGS. 19A-19D. Deconvoluted mass spectrum from RP-HPLC-ESI-MS data. (FIG. 19A) A mixture of GFP-EcTeCH, GFP-LjTeCH, and GFP-HpTeCH. Calc: GFP-EcTeCH, 27,947 Da; GFP-LjTeCH, 28,113 Da; GFP-HpTeCH, 28,096 Da. Observed: GFP-EcTeCH, 27,948 Da; GFP-LjTeCH, 28,114 Da; GFP-HpTeCH, 28,096 Da. (FIG. 19B) A mixture of GFP-EcTeCH, GFP-LjTeCH, and GFP-HpTeCH modified with EcM ccB. Calc: GFP-EcTeCH, 27,947 Da; GFP-LjTeCH, 28,113 Da; GFP-HpTeCH, 28,096 Da; GFP-EcTeCH-Mes, 28,071 Da; GFP-LjTeCH-Mes, 28, 237; GFP-HpTeCH-Mes, 28,220 Da. Observed: GFP-EcTeCH-Mes, 28,072 Da; GFP-LjTeCH, 28,114 Da; GFP-HpTeCH, 28,095 Da. (FIG. 19C) A mixture of GFP-EcTeCH, GFP-LjTeCH, and GFP-HpTeCH modified with LjM ccB. Calc: GFP-EcTeCH, 27,947 Da; GFP-LjTeCH, 28,113 Da; GFP-HpTeCH, 28,096 Da; GFP-EcTeCH-Mes, 28,071 Da; GFP-LjTeCH-Mes, 28,237; GFP-HpTeCH-Mes, 28,220 Da. Observed: GFP-EcTeCH, 27,948 Da; GFP-LjTeCH-Mes, 28,238 Da; GFP-HpTeCH, 28,096 Da. (FIG. 19D) A mixture of GFP-EcTeCH, GFP-LjTeCH, and GFP-HpTeCH modified with HpM ccB. Calc: GFP-EcTeCH, 27,947 Da; GFP-LjTeCH, 28,113 Da; GFP-HpTeCH, 28,096 Da; GFP-EcTeCH-Mes, 28,071 Da; GFP-LjTeCH-Mes, 28,237; GFP-HpTeCH-Mes, 28,220 Da. Observed: GFP-EcTeCH, 27,948 Da; GFP-LjTeCH-Mes, 28,114 Da; GFP-HpTeCH-Mes, 28,220 Da.

FIGS. 20A-20E. M ccB enables ATP-dependent thi oester formation and regeneration for high-yield enzyme-catalyzed expressed protein ligation. (FIG. 20A) Enzyme-catalyzed expressed protein ligation is limited by subtiligase-catalyzed hydrolysis of the thioester substrate, generating a dead-end product. We used M ccB for ATP-dependent thioester formation and regeneration, enabling reactivation of the dead-end hydrolytic product. (FIG. 20B) High yield one-pot M ccB- and subtiligase-catalyzed ATP-dependent peptide ligation to GFP-TeCH. (FIG. 20C) MccB- and subtiligase-catalyzed ATP-dependent peptide ligation to TeCH-tag fusions of MBP, the catalytic domain of protein tyrosine phosphatase 1B (PTP1B 1-321), protein L, an α-GFP recombinant antibody, and an EGFR-targeting affibody. The * indicates an α-gluconylated form of protein L that is an artifact of His-tag purification. (FIG. 20D) MccB- and subtiligase-catalyzed peptide ligation and strain promoted azide-alkyne cycloaddition were used to synthesize α-GFP rAb-Cy3 for staining of a HEK 293T cell line engineered for doxycycline-inducible expression of cell surface GFP. (FIG. 20E) Dual N- and C-terminal labeling of TeCH-tagged MBP with 5-FAM-LPETGG (SEQ ID NO: 60) and AFAGAGS-azidoA la (SEQ ID NO: 59) using eSrtA and M B P/subtiligase under one-pot (center) or telescoping (right) conditions.

FIGS. 21A-21Y. Deconvoluted mass spectrum from RP-HPLC-ESI-MS data. (FIG. 21A) GFP-EcTeCH ligated to AFAGAGSazK (SEQ ID NO: 57). Calc: GFP-EcTeCH-AFAGAGSazK, 28,662 Da. Observed: 28,662 Da. The peak marked with a * corresponds to subtiligase. (FIG. 21B) GFP-HpTeCH ligated to AFA GA GSazA (SEQ ID NO: 59). Calc: 28,768 Da. Observed: 28,769 Da. The peak marked with a * corresponds to subtiligase. (FIG. 21C) GFP-LjTeCH ligated to AFAGAGSazA. Calc: 28,785 Da. Observed: 28,786 Da. The peak marked with a * corresponds to subtiligase. (FIG. 21D) MBP-EcTeCH ligated to AFAGAGSazK. Calc: 44,314 Da. Observed: 44,316 Da. The peak marked with a * corresponds to subtiligase. (FIG. 21E) PTP1B 1-321-TeCH ligated to AFAGAGSazK. Calc: 39,088 Da. Observed: 39,090 Da. The peak marked with a * corresponds to subtiligase. (FIG. 21F) Anti-GFP recombinant antibody (aGFP rAb)-TeCH ligated to AFA GAGSazK. Calc: 48,715 Da. Observed: 48,716 Da. (FIG. 21G) Protein L-TeCH ligated to AFAGAGSazK. Calc: 43,394 Da (unmodified), 43,567 Da (N-gluconylated). Observed: 43,396 Da, 43,581 Da. (FIG. 21H) zEGFR-TeCH ligated to AFA GA GSazK. Calc: 11,286 Da (M et excised). Observed: 11,285 Da. (FIG. 211) Anti-GFP recombinant antibody (aG FP rA b) ligated to AFA GAGSazK and modified with DBCO-Cy3 by strain-promoted azide-alkyne cycloaddition (SPAAC). Calc: 49,698 Da. Observed: 49,699 Da. (FIG. 21J) GS-GFP-LPETGG. Calc: 27,814 Da. Observed: 27,814 Da. (FIG. 21K) GS-GFP-LPETGG treated with 2.5 mM eSrtA (no GGG) control. Calc: unmodified, 27,814 Da; cyclized, 27,681 Da. Observed: 27,681 Da, 27,814 Da. (FIG. 21L) RP-HPLC-ESI-MS data for GS-GFP-L PETGG treated with 2.5 mM eSrtA and 5 mM GGG. Calc: unmodified, 27,814 Da; ligation product, 27,870 Da; cyclized, 27,681 Da. Observed: 27,682 Da, 27,814 Da. (FIG. 21M) GS-GFP-LPETGG treated with 2.5 mM eSrtA and 10 mM GGG. Calc: unmodified, 27,814 Da; ligation product, 27,870 Da; cyclized, 27,681 Da. Observed: 27,681 Da, 27,814 Da. (FIG. 21N) MV-GFP-LPETGG. Calc: unmodified, 28,408 Da; ligation product, 27,762 Da; cyclized, 27,557 Da. Observed: 28,408 Da. (FIG. 21O) MV-GFP-LPETGG treated with 2.5 mM eSrtA (no GGG control). Calc: unmodified, 28,408 Da; ligation product, 27,762 Da; cyclized, 27,557 Da. Observed: 28,409 Da. (FIG. 21P) MV-GFP-LPETGG treated with 2.5 mM eSrtA and 5 mM GGG. Calc: unmodified, 28,408 Da; ligation product, 27,762 Da; cyclized, 27,557 Da. Observed: 27,762 Da. (FIG. 21Q) Dual N- and C-terminal labeling reaction with eSrtA and M ccB/subtiligase. Calc: unmodified, 43,599 Da; sortase-modified, 44,399 Da; 43,724 Da, M es thioester only; M ccB/subiligase-modified, 44,274 Da; 44,523 Da, sortase-modified M es thioester; 45,073 Da, dual labeled. Observed: 45,072 Da; 44,524 Da; 44,400 Da. (FIG. 21R) Dual N- and C-terminal labeling reaction with eSrtA and M ccB/subtiligase-no subtiligase control. Calc: unmodified, 43,599 Da; sortase-modified, 44,399 Da; 43,724 Da, M es thioester only; M ccB/subiligase-modified, 44,274 Da; 44,523 Da, sortase-modified M es thioester; 45,073 Da, dual labeled. Observed: 44,524 Da; 44,399 Da. (FIG. 21S) Dual N- and C-terminal labeling reaction with eSrtA and M ccB/subtiligase no M ccB control. Calc: unmodified, 43,599 Da; sortase-modified, 44,399 Da; 43,724 Da, Mes thioester only; M ccB/subiligase-modified, 44,274 Da; 44,523 Da, sortase-modified M es thioester; 45,073 Da, dual labeled. Observed: 44,400 Da. (FIG. 21T) Dual N- and C-terminal labeling reaction with eSrtA and MccB/subtiligase-no eSrtA control. Calc: unmodified, 43,599 Da; sortase-modified, 44,399 Da; 43,724 Da, Mes thioester only; M ccB/subiligase-modified, 44,274 Da; 44,523 Da, sortase-modified M es thioester; 45,073 Da, dual labeled. Observed: 44,274 Da. (FIG. 21U) Dual N- and C-terminal labeling reaction with eSrtA and M ccB/subtiligase-no enzymes control. Calc: unmodified, 43,599 Da; sortase-modified, 44,399 Da; 43,724 Da, Mes thioester only; M ccB/subiligase-modified, 44,274 Da; 44,523 Da, sortase-modified M es thioester; 45,073 Da, dual labeled. Observed: 44,274 Da. (FIG. 21V) Dual N- and C-terminal labeling reaction with eSrtA and M ccB/subtiligase under telescoping conditions. Calc: unmodified, 43,599 Da; sortase-modified, 44,399 Da; 43,724 Da, Mes thioester only; M ccB/subiligase-modified, 44,274 Da; 44,523 Da, sortase-modified M es thioester; 45,073 Da, dual labeled. Observed: 45,072 Da. (FIG. 21W) Dual N- and C-terminal labeling reaction with eSrtA and M ccB/subtiligase under telescoping conditions-no subtiligase control. Calc: unmodified, 43,599 Da; sortase-modified, 44,399 Da; 43,724 Da, Mes thioester only; M ccB/subiligase-modified, 44,274 Da; 44,523 Da, sortase-modified M es thioester; 45,073 Da, dual labeled. Observed: 44,524 Da. (FIG. 21X) Dual N- and C-terminal labeling reaction with eSrtA and M ccB/subtiligase under telescoping conditions-no MccB control. Calc: unmodified, 43,599 Da; sortase-modified, 44,399 Da; 43,724 Da, M es thioester only; M ccB/subiligase-modified, 44,274 Da; 44,523 Da, sortase-modified M es thioester; 45,073 Da, dual labeled. Observed: 44,400 Da. (FIG. 21Y) Dual N- and C-terminal labeling reaction with eSrtA and M ccB/subtiligase under telescoping conditions-no eSrtA control. Calc: unmodified, 43,599 Da; sortase-modified, 44,399 Da; 43,724 Da, M es thioester only; M ccB/subiligase-modified, 44,274 Da; 44,523 Da, sortase-modified M es thioester; 45,073 Da, dual labeled. Observed: 44,274 Da.

FIG. 22. Enzyme-catalyzed expressed protein ligation (EPL) with GFP-TeCH and A la-Phe dipeptide. (Panel A) Scheme of one pot reaction for GFP-TECH thioesterficaition followed by subtiligase-catalyzed ligation with AlaPhe dipeptide. (Panel B) HPLC-MS quantification of AlaPhe ligation product with 5 mM AlaPhe. (Panel C) Representative deconvoluted traces of HPLC-MS intact protein analysis. HPLC-MS analysis of Mesna thioesterification and Ala-Phe ligation with 5 mM Ala-Phe. (Panel D) HPLC-MS quantification of A laPhe ligation product with titrated A la-Phe concentrations. (Panel E) Representative deconvoluted traces of HPLC-MS intact protein analysis of GFP-TeCH expressed protein EPL with decreasing concentrations of A la-Phe.

FIG. 23. M ccB homologs from L. johnsonii and H. pylori can be used in combination with subtiligase for enzyme-catalyzed expressed protein ligation. (Panel A) LjM ccB- and subtiligase-catalyzed ATP-dependent peptide ligation of Ala-Phe (left) or AFAGAGS-azAla (SEQ ID NO: 59) (right) to GFP-LjTeCH. (Panel B) HpM ccB- and subtiligase-catalyzed ATP-dependent peptide ligation of Ala-Phe (left) or AFAGAGS-azA la (right) to GFP-HpTeCH.

FIG. 24. ESI-M S spectrum for AFAGAGSazA (SEQ ID NO: 59)C-terminal amide. Calc m/z: [M+H]+, 691.3276; [M+2H]2+, 346.1677. Observed m/z: 691.3323, 346.168.

FIG. 25. Comparison of M ccB/subtiligase-catalyzed and eSrtA-catalyzed C-terminal protein modification. (Panel A) M ccB/subtiligase-catalyzed C-terminal peptide ligation to GS-GFP-TeCH. In the absence of peptide nucleophile, M ccB and subtiligase catalyze GS-GFP cyclization, but this reaction is efficiently suppressed in the presence of 5 mM Ala-Phe. (Panel B) eSrtA-catalyzed C-terminal modification of GS-GFP-LPETGG. In the absence of nucleophile, eSrtA catalyzes GFP cyclization that cannot be completely suppressed even in the presence of 10 mM GGG peptide. (C) eSrtA cyclization is suppressed by removing the N-terminal GS sequence at the N terminus of GS-GFP-LPETGG.

FIG. 26. Dual N- and C-terminal labeling of GS-MBP-TeCH using eSrtA and M ccB/subtiligase. (Panel A) Scheme for dual N- and C-terminal label of GS-MBP-TeCH with M ccB/subtiligase and eSrtA. The magenta circle represent azidoA la and the cyan circle represents 5-FAM. (Panel B) Telescoping one-pot dual labeling of GS-MBP-TeCH with eSrtA and M ccB/subtiligase. (Panel C) Concurrent one-pot dual labeling of GS-MBP-TeCH with eSrtA and M ccB/subtiligase.

FIGS. 27A-27E. Enzymatic synthesis of ubiquitin-derived peptide thioesters using an MccB homolog with relaxed substrate specificity. (FIG. 27A) Synthetic ubiquitin-derived peptide C-terminal thioesters are substrates for Ubc9 in the lysine acylation with conjugating enzymes (LA CE) strategy. (FIG. 27B) M ccB-catalyzed synthesis of peptide C-terminal thioesters. (FIG. 27C) M ccB from Histophilus somni (cyan, HsM ccB) acts on a basic substrate and exhibited lower substrate specificity in our screen of M ccB homologs. (FIG. 27D) HsM ccB efficiently converts Ubc9 substrate peptides for LACE to Mes thioesters (left), while AcCysNHMe (right) was a less effective thiol donor. (FIG. 27E) Application of M ccB-generated thioesters for acylation of an internal lysine side chain in LACE-tagged GFP. Left, acylation using 300 mM LRLRGG-Mes thioester. Right, acylation using 750 mM M ccB-generated MLGLRGG-M es thioester.

FIG. 28. Combining HsM ccB-catalyzed peptide thioester synthesis with Ubc9-catalyzed lysine acylation. (Panel A) Scheme for lysine acylation using an HsM ccB-generated thioester and GFP with an internal minimal LACE tag sequence (IK QE). (Panel B) Scheme for lysine acylation using an HsM ccB-generated thioester and GFP with a full length LACE tag sequence (PRKVIKMESEE; SEQ ID NO: 92). (Panel C) Optimization of peptide thioester concentration in LACE reactions. (Panel D) Optimization of thiol concentration at pH 7.6. Excess thiol suppresses the LACE reaction. (Panel E) Optimization of thiol concentration at pH 8.0. Excess thiol suppresses the LACE reaction, which proceeds to higher yield at pH 8.0 compared to 7.6.

FIGS. 29A-29C. Deconvoluted mass spectrum from RP-HPLC-ESI-MS data. (FIG. 29A) Ubc9-catalyzed lysine acylation of GFP with an internal LACE tag after D 173-no peptide control. Calc: unmodified, 28,517 Da. Observed: 28,517 Da. The later-eluting peak corresponds to Ubc9. (FIG. 29B) Ubc9-catalyzed lysine acylation of GFP with an internal LACE tag after D173-300 mM LRLRGG-Mes. Calc: unmodified, 28,517 Da; lysine-acylated, 29,170 Da. Observed: 29,170 Da. The later-eluting peak corresponds to Ubc9. (FIG. 29C) Ubc9-catalyzed lysine acylation of GFP with an internal LACE tag after D 173 750 mM M ccB-generated MLGLRGG-Mes (SEQ ID NO: 88). Calc: unmodified, 28,517 Da; lysine-acylated, 29,202 Da. Observed: 29,202 Da. The later-eluting peak corresponds to Ubc9.

FIGS. 30A-30G. Deconvoluted mass spectrum from RP-HPLC-ESI-MS data. (FIG. 30A) E. coli MccB. Calc: 41,740 Da (Met loss). Observed: 41,741 Da, 41,920 Da (N-gluconylation). (FIG. 30B) H. pylori M ccB. Calc: 42,664 Da (M et loss). Observed: 41,664 Da, 42,843 Da (N-gluconylation). (FIG. 30C) L. johnsonii MccB. Calc: 42,349 Da (M et loss). Observed: 41,741 Da, 41,920 Da (N-gluconylation). (FIG. 30D) H. somni MccB. Calc: 42,819 Da (M et loss). Observed: 42,821 Da, 42,999 Da (N-gluconylation), 43,079 Da. (FIG. 30E) Ubc9. Calc: 20,632 Da (M et loss). Observed: 20,632 Da, 20,810 Da (N-gluconylation). (FIG. 30F) Subtiligase. Calc: 28,546 Da (mature subtiligase). Observed: 28,547 Da. (FIG. 30G) eSrtA. Calc: 17,853 Da. Observed: 17,853 Da.

FIG. 31. Enzyme-coupled pyrophosphate assay for detection of M ccB-catalyzed pyrophosphate release. M ccB-catalyzes release of pyrophosphate, which is converted to phosphate by inorganic pyrophosphatase (PPiase). Purine nucleoside phosphorylase (PN Pase) uses phosphate as a substrate to convert 2-amino-6-mercapto-7-methylpurine ribonucleoside (M ESG) to ribose 1-phosphate and 2-amino-6-mercapto-7-methylpurine, which absorbs at 360 nm.

### DETAILED DESCRIPTION

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the present invention shall include the corresponding plural characteristic or limitation, and vice-versa, unless otherwise specified or clearly implied to the contrary by the context in which the reference is made. The indefinite articles “a” and “an” mean “one or more.” The word “or” is used inclusively and should be read “and/or.”

All combinations of method or process steps as used herein can be performed in any order, unless otherwise specified or clearly implied to the contrary by the context in which the referenced combination is made.

The methods of the present invention can comprise, consist of, or consist essentially of the essential elements and limitations of the method described herein, as well as any additional or optional ingredients, components, or limitations described herein or otherwise useful in molecular biology, organic chemistry, and/or genetic engineering. The disclosure provided herein suitably may be practiced in the absence of any element which is not specifically disclosed herein.

It is understood that the disclosure is not confined to the particular ingredients, compositions of matter, or steps herein illustrated and described, but embraces such modified forms thereof as come within the scope of the claims.

The term “homologous sequences” as used herein refers to a polynucleotide or polypeptide sequence having, for example, about 100%, about 99% or more, about 98% or more, about 97% or more, about 96% or more, about 95% or more, about 94% or more, about 93% or more, about 92% or more, about 91% or more, about 90% or more, about 88% or more, about 85% or more, about 80% or more, about 75% or more, about 70% or more, about 65% or more, about 60% or more, about 55% or more, about 50% or more, about 45% or more, or about 40% or more sequence identity to another polynucleotide or polypeptide sequence when optimally aligned for comparison. In particular versions, homologous sequences can retain the same type and/or level of a particular activity of interest. In some embodiments, homologous sequences have between 85% and 100% sequence identity, whereas in other embodiments there is between 90% and 100% sequence identity. In particular embodiments, there is 95% and 100% sequence identity.

“Homology” refers to sequence similarity or sequence identity. Homology is determined using standard techniques known in the art (see, e.g., Smith and Waterman, Adv. Appl. Math., 2:482, 1981; Needleman and Wunsch, J. Mol. Biol., 48:443, 1970; Pearson and Lipman, Proc. Natl. Acad. Sci. USA 85:2444, 1988; programs such as GAP, BESTFIT, FASTA, and TFASTA in the Wisconsin Genetics Software Package (Genetics Computer Group, Madison, Wisconsin); and Devereux et al., Nucl. Acid Res., 12:387-395, 1984). A non-limiting example includes the use of the BLAST program (Altschul et al., “Gapped BLAST and PSI-BLAST: a new generation of protein database search programs,” Nucleic Acids Res. 25:3389-3402, 1997) to identify sequences that can be said to be “homologous.” More recent versions include sub-programs such as blastp for protein-protein comparisons, blastn for nucleotide-nucleotide comparisons, tblastn for protein-nucleotide comparisons, and blastx for nucleotide-protein comparisons, and with parameters as follows: Maximum number of sequences returned 10,000 or 100,000; E-value (expectation value) of 1e-2 or 1e-5, word size 3, scoring matrix BLOSUM 62, gap cost existence 11, gap cost extension 1, may be suitable. An E-value of 1e-5, for example, indicates that the chance of a homologous match occurring at random is about 1 in 10,000, thereby marking a high confidence of true homology.

The term “identical,” in the context of two polynucleotide or polypeptide sequences, means that the residues in the two sequences are the same when aligned for maximum correspondence, as measured using a sequence comparison or analysis algorithm such as those described herein. For example, if when properly aligned, the corresponding segments of two sequences have identical residues at 5 positions out of 10, it is said that the two sequences have a 50% identity. M ost bioinformatic programs report percent identity over aligned sequence regions, which are typically not the entire molecules. If an alignment is long enough and contains enough identical residues, an expectation value can be calculated, which indicates that the level of identity in the alignment is unlikely to occur by random chance.

Disclosed herein is an enzyme-substrate pair that can be used as a tool for C-terminal modification, including thioesterification, of proteins and peptides. The tool is based on the E1-like enzyme M ccB, whose native function involves the formation of a C-terminal phosphoramidate linkage in microcin C7 biosynthesis. It is demonstrated that M ccB has a latent capacity to catalyze C-terminal O-A M Pylation on non-native substrates, resulting in a C-terminal electrophile that can react with a variety of exogenous nucleophiles, including hydrazines, alkoxyamines, amines, and thiols. This reaction results in a diverse set of C-terminal functional groups. Also disclosed herein is a sequence tag, namely Thioesterification C-terminal Handle (TeCH-tag), that enables M ccB-catalyzed, ATP-dependent mdification of protein and peptide C-termini.

The E1-like enzyme superfamily is comprised of structurally and mechanistically related proteins that function in biological processes ranging from ubiquitination to biosynthesis of diverse metabolites including thiamin, molybdopterin, cysteine, and ribosomally synthesized and post-translationally modified peptide (RiPP) natural products. Despite their functional diversity, E1-like enzymes are proposed to share a common mechanistic step involving O-AM Pylation of a C-terminal carboxylate to generate a reactive acyl-AMP mixed anhydride electrophile. Based on its capacity to react with diverse nucleophiles, this shared intermediate provides biological systems with access to protein and peptide C-terminal thioesters, thiocarboxylates, succinimides, and (iso) peptide bonds. As described herein, O-AM Pylated intermediates can be integrated into the chemical biology toolbox for protein synthesis and semisynthesis.

E1-like superfamily enzymes are discussed in detail in Burroughs et al. “Natural history of the E 1-like superfamily: implication for adenylation, sulfur transfer and ubiquitin conjugation”, Proteins, 75 (4), pp. 895-910, 2009.

The enzyme used in the methods described herein is an E1-like superfamily enzyme which catalyzes O-AM Pylation of a C-terminal carboxylate group to provide a reactive acyl-AMP mixed anhydride electrophile. An exemplary class of E1-like superfamily enzyme is M ccB, which natively functions in the biosynthesis of microcin C7 by modifying a peptide substrate M ccA through ATP-dependent adenylation and phosphoramidate bond formation.

Exemplary M ccB enzymes include Escherichia coli M ccB (SEQ ID NO: 1; UniProt Q2KKH8), Helicobacter pylori MccB (SEQ ID NO: 2; RefSeq Accession No. WP_033777882.1), Lactobacillus johnsonii M ccB (SEQ ID NO: 3; RefSeq Accession No. WP 113886641.1), Histophilus somni MccB (SEQ ID NO: 4; RefSeq Accession No. WP 075293582.1), or a catalytically active homolog thereof.

E. coli M ccB
(SEQ ID NO: 1)
MDYILGRYVKIARYGSGGLVGGGGKEQYVEDLALWENIIKTAYCF
ITPSSYTAALETVNIPEKDFSNCFRFLKENFFIIPSEYNNSTENN
RYSRNFLHYQSYGANPVLVQDKLKDAKVVILGCGGIGNHVSVILA
TSGIGEIILIDNDQIENTNLTRQVLFSENDVGKNKTEVIKRELLK
RNSEISVSEIALNINDYTDLHKVPEADIWVVSADHPFNLINWVNK
YCVRANQPYINAGYVNDIAVFGPLYVPGKTGCYECQKVVADLYGS
EKENIDHKIKLINSRFKPATFAPVNNVAAALCAADVIKFIGKYSE
PLSLNKRIGIWSDEIKIHSQNMGRSPVCSVCGNRM
H. pylori M ccB
(SEQ ID NO: 2)
MQWYQTSFSACVGQTDTENIIGLGTYQYCVDHNEFEKSLKLLVFL
RMKKRMAEIKSFMETSKIEHNIFDKLVANKLITSFILNPNDEQNF
KNHLFIDLMSSKPELTIDNFKRTIFIIIGOGGIGNFVSYALASFY
PKKLILLDKDTVDFSNLNRQFLFDKNYISQYKTSAIKQALSSRFS
INIETVDDFASEDNLEEIFSKHKKENLFGIVSGDNPNTVQLATRF
FCKCRIPFLNIGYLNDISLIGPFYIPSLSCCPFCHNSFALDDKKD
GDENLDICLI
L. johnsonii M ccB
(SEQ ID NO: 3)
MFYKTSYLATGGCSNHQGILGVGTKQYFVSEADYLKSLKILDFLL
NKKTYDEVIKFCEKNNINKSIFDTLVEHNLIVKENLYVEKKDDLN
FKNKLYFHALGLNGNALAKEFADTTFVIVGOGGIGNFISFAIGSL
SPRKIELIDGDKIEKSNLNRQFLFTENDIGKYKVDVLKKNLVERN
NKLSISEYKEYVSKEVLHNIFEQNKKNKTLVILSGDSFSALSLTA
KACVKSEIPFLNIGYLNDISAIGPFYIPGISSCPFCHNALSISDD
ISSGHNESKILEDRINANNEAPSSFTNNALAASMGIADIIEFLSH
NYERINSLNKRFGINSATFEKYVLEVNRDRKCEICSHGE
H. somni M ccB
(SEQ ID NO: 4)
MKYITSKHVFFDYLNENEFVIGIGSNQEITNNKDYFNNCLNLCYF
CINPKSISEILSFIKDNNIDILYFDKMKKMKFITKEIIDFNDRYS
RNHLYYNALGYKIYDIQNKISKSHILIVGAGGIGNICSYLLGTIG
IKKLSIIDDDIVEESNLNRQFLFREKDINKNKVETIKRELLSIRK
DIIIDIFPEKLNKSILDKISQIDLVICSADDEYCIDMINEFCCFN
KIPLINVGYLNDISVIGPFYIPKLEYSCCLCCDKSIYLENDVIDE
KVKKIKSVTKAPSTIINNFFAGAMLGSELIKFFARDYKSMQSINS
VIGIHNKNFKYEEIKLAKNYNCKYCGVNNETL

The amino acid sequence of M ccB may comprise an amino acid sequence at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NOs: 1-4.

Homologs of M ccB also include Bacillus subtilis moeB (UniProt 031702), Bacillus cereus M oeB (UniProt Q816U 3), and others. See, for example, Zukher et al. “Reiterative synthesis by the ribosome and recognition of the N-terminal formyl group by biosynthetic machinery contribute to evolutionary conservation of the length of antibiotic microcin C peptide precursor.” mBio 10, e00768-19, 2019; Bantysh et al. “Enzymatic synthesis of bioinformatically predicted microcin C-like compounds encoded by diverse bacteria.” mBio 5, e01059-14, 2014. See also Table 1 below, which is derived from Zukher et al. (2019) and provides additional M ccB homologs.

In one aspect, the substrate for the E1 superfamily enzyme is a peptide. In the case of MccB, the substrate is the peptide MccA. The specific substrate sequence varies depending on the source organism, as different M ccB homologs recognize distinct peptide substrates. For example, while E. coli M ccB recognizes the heptapeptide MRTGNAN (SEQ ID NO: 18), H. pylori MccB recognizes MKLSYRN (SEQ ID NO: 21), and L. johnsonii M ccB recognizes MHRIMKN (SEQ ID NO: 23). H. somni MccB recognizes MRGRRLN (SEQ ID NO: 22) and can also, to some extent, recognize the M ccA peptides of E. coli, H. pylori, and L. johnsonii (SEQ ID NOs: 18, 21, and 23).

M ccA is typically a short peptide containing a C-terminal asparagine residue which is essential for enzymatic adenylation by an M ccB-like enzyme, and optionally containing an N-terminal methionine residue which can be formylated to enhance enzyme recognition and modification. Many M ccB enzyme homologs recognize heptapeptide substrates of the form MXXXXXN, wherein “X” represents any amino acid. Some MccB-like enzymes recognize longer sequences, which retain the conserved methionine and asparagine termini. These substrate sequences take the form M (X n) N, wherein “X” is any amino acid, and the subscript “n” represents an integer from 6 to 54. Some substrates may contain a valine at the N-terminal instead of methionine. See Table 1 for additional substrate sequences recognized by M ccB homologs.

TABLE 1
Substrate peptide sequences of M ccB-like proteins.

| M ccB-like protein | | |
|---|---|---|
| accession number | Organism | Substrate peptide sequence |
| WP_090821928.1 | Arthrobacter | MLKLKRLAKRRSKLNFVFN |
| | | (SEQ ID NO: 5) |
| WP_011690819.1 | Arthrobacter sp. FB 24 | VLKLKRLTKRRSKLNFVFN |
| | | (SEQ ID NO: 6) |
| WP_017197322.1 | Arthrobacter sp. M 2012083 | VLKLKRLAKRRSKLNFVFN |
| | | (SEQ ID NO: 7) |
| WP_104173467.1 | Arthrobacter sp. Y 81 | VLKLKRLAKRRSKLNFVFN |
| | | (SEQ ID NO: 7) |
| WP013352374.1 | Bacillus amyloliquefaciens | MLKIRKVKIVRAQNGHYTN |
| | | (SEQ ID NO : 8) |
| WP_005866101.1 | Bartonella alsatica | MDHIGFN (SEQ ID NO: 9) |
| WP_038525236.1 | Bartonella henselae | MDHPEFN (SEQ ID NO: 10) |
| WP_010704712.1 | Bartonella vinsonii | MDHRGFN (SEQ ID NO: 11) |
| WP_006926230.1 | Bartonella washoeensis | MDHIGEN (SEQ ID NO: 9) |
| WP_025408860.1 | Borrelia coriaceae | MRGKTLN (SEQ ID NO: 12) |
| WP_012539709.1 | Borrelia duttonii | MRGKTLN (SEQ ID NO: 12) |
| WP_025407054.1 | Borrelia hermsii | MRGKTLN (SEQ ID NO: 12) |
| WP_012622144.1 | Borreliella garinii | MLGKNCN (SEQ ID NO: 13) |
| WP_113865755.1 | Brenneria salicis | MDHSMNN (SEQ ID NO: 14) |
| WP_088206152.1 | Chlamydiales bacterium | MDTVTTN (SEQ ID NO: 15) |
| | SCGC AG-110-P3 | |
| WP_066070088.1 | Clostridium paradoxum | MRLIIRN (SEQ ID NO: 16) |
| WP_019844494.1 | Dickeya zeae | MISVSSN (SEQ ID NO: 17) |
| WP_114979778.1 | Enterobacter sp. 9-2 | MRTGNAN (SEQ ID NO: 18) |
| WP_001371842.1 | Escherichia coli | MRTGNAN (SEQ ID NO: 18) |
| WP_032834209.1 | Gardnerella vaginalis | MHVTPSN (SEQ ID NO: 19) |
| WP_005714716.1 | Glaesserella parasuis | MRGRRVN (SEQ ID NO: 20) |
| WP_033777882.1 | Helicobacter pylori | MKLSYRN (SEQ ID NO: 21) |
| WP_075293582.1 | Histophilus somni | MRGRRLN (SEQ ID NO: 22) |
| WP_006729676.1 | Lactobacillus iners | MHVTPSN (SEQ ID NO: 19) |
| WP_113886641.1 | Lactobacillus johnsonii | MHRIMKN (SEQ ID NO: 23) |
| WP_004048748.1 | Lactobacillus murinus | MSLMIKN (SEQ ID NO: 24) |
| WP_087612460.1 | Marinomonas sp. QM 202 | MIARLLN (SEQ ID NO: 25) |
| WP_029102054.1 | Moraxella caprae | MIAMAHN (SEQ ID NO: 26) |
| WP_120721051.1 | Moraxella catarrhalis | MISINPFN (SEQ ID NO: 27) |
| WP_106581501.1 | M urinocardiopsis flavida | MNGDGDN (SEQ ID NO: 28) |
| WP_079608811.1 | Mycobacteroides abscessus | MKTLAVKIRRIVRAQGSQKSG |
| | | SN (SEQ ID NO: 29) |
| WP_017605001.1 | Nocardiopsis alkaliphila | MNDDGSN (SEQ ID NO: 30) |
| WP_061080142.1 | Nocardiopsis dassonvillei | MNDDGNN (SEQ ID NO: 31) |
| WP_073382941.1 | Nocardiopsis flavescens | VNDDGNN (SEQ ID NO: 32) |
| WP_017573747.1 | Nocardiopsis halotolerans | VNDDGNN (SEQ ID NO: 32) |
| WP_017574752.1 | Nocardiopsis kunsanensis | MNDDGNN (SEQ ID NO: 31) |
| WP_067604483.1 | Nocardiopsis listeri | MNDDGNN (SEQ ID NO: 31) |
| WP_017597350.1 | Nocardiopsis lucentensis | MNDDGNN (SEQ ID NO: 31) |
| WP_087097089.1 | Nocardiopsis sp. J B 363 | MNDDGNN (SEQ ID NO: 31) |
| WP_087098035.1 | Nocardiopsis sp. J B 363 | MRRISMTPVRKVKGSTTVVA |
| | | N (SEQ ID NO: 33) |
| WP_047870366.1 | Nocardiopsis sp. RV 163 | VNDDGNN (SEQ ID NO: 32) |
| WP_017563298.1 | Nocardiopsis | VNDDGNN (SEQ ID NO: 32) |
| | synnemataformans | |
| WP_017582911.1 | Nocardiopsis valliformis | MNDDGNN (SEQ ID NO: 31) |
| WP_017608646.1 | Nocardiopsis xinjiangensis | MNDDGNN (SEQ ID NO: 31) |
| WP_089562621.1 | Pantoea | MDHSMNN (SEQ ID NO: 14) |
| WP_044203004.1 | Pectobacterium | MDHSMNN (SEQ ID NO: 14) |
| | carotovorum | |
| WP_012822359.1 | Pectobacterium parmentieri | MEHTTNN (SEQ ID NO: 34) |
| WP_058589644.1 | Photorhabdus laumondii | MISSGSN (SEQ ID NO: 35) |
| WP_051769631.1 | Photorhabdus temperata | MISIASN (SEQ ID NO: 36) |
| WP_078806649.1 | Pilibacter termitis | MAGPLRN (SEQ ID NO: 37) |
| WP_122251128.1 | Pseudomonas marginalis | MISINPEN (SEQ ID NO: 27) |
| WP_124433381.1 | Pseudomonas orientalis | MISINPFN (SEQ ID NO: 27) |
| WP_081585397.1 | Pseudomonas sp. PAM C | MISINPEN (SEQ ID NO: 27) |
| | 26793 | |
| WP_052447771.1 | Serratia symbiotica | MIGGLNN (SEQ ID NO: 38) |
| WP_096546674.1 | Staphylococcus delphini | MLEIQEVKEIEGRSAHVSN |
| | | (SEQ ID NO: 39) |
| WP_107597431.1 | Staphylococcus gallinarum | MLEIKEVKEIEGRSKNLSN |
| | | (SEQ ID NO: 40) |
| WP_107623312.1 | Staphylococcus hominis | MLEIKEIKDVQANSVNVSN |
| | | (SEQ ID NO: 41) |
| WP_111501223.1 | Streptacidiphilus pinicola | MLKIVKKPVVKTDSIMVHN |
| | | (SEQ ID NO: 42) |
| WP073508338.1 | Streptobacillus notomytis | VIFTVVKN (SEQ ID NO: 43) |
| WP011227610.1 | Streptococcus thermophilus | MKGTILN (SEQ ID NO: 44) |
| WP_076995516.1 | Streptococcus azizii | MIGPIQN (SEQ ID NO: 45) |
| WP_024376108.1 | Streptococcus suis | MVGPVQN (SEQ ID NO: 46) |
| WP_011365433.1 | Synechococcus sp. CC 9605 | MTQPNDRQLSNEELSDVAAG |
| | | LFRRTFFKPRTSRKTLLQPKRL |
| | | DKVAKNQLWADMMN (SEQ |
| | | ID NO : 47) |
| WP_084012769.1 | Thermobifida halotolerans | MNGDGSN (SEQ ID NO: 48) |
| WP_083847841.1 | Turneriella parva | MKIEKTAKKITRTGGAN (SEQ |
| | | ID NO : 49) |
| WP_049599515.1 | Yersinia | MHQSEIKLTKRLKIKRVDVNK |
| | pseudotuberculosis | VKEQQKKVLECGAATCGGGS |
| | complex | N (SEQ ID NO: 50) |
| WP_042820409.1 | Yersinia wautersii | MDHTTSN (SEQ ID NO: 51) |

The present disclosure demonstrates that when the C-terminal asparagine of M ccA is replaced by another amino acid (such as alanine, glycine, serine, or threonine), M ccB catalyzes C-terminal O-AM Pylation in the absence of the C-terminal asparagine. The resulting C-terminally O-A M Pylated M ccA can be captured by exogenous nucleophiles to form diverse C-terminal functional groups. Thus, when the modified M ccA peptide is fused to the C-terminus of a polypeptide of interest, the fusion polypeptide is a substrate for 0-AM Pylation by M ccB. In a specific aspect, the polypeptide of interest is not naturally fused to the modified M ccA peptide.

This novel substrate is referred to herein as a Thioesterification C-terminal Handle (TeCH-tag). The TeCH-tag takes the general sequence formula of (X) nX′, wherein X can be any amino acid, n is an integer from 6 to 55, and X′ is an amino acid other than asparagine (N). In a specific aspect, TeCH-tag sequences are heptapeptides that contain a C-terminal glycine (G) which enables them to function as substrates for O-AM Pylation and subsequent modification. For example, an E. coli TeCH-tag sequence disclosed herein is MRTGNAG (ECM ccA-N 7G; SEQ ID NO: 52); a H. pylori TeCH-tag sequence disclosed herein is M KLSY RG (HpM ccA-N 7G; SEQ ID NO: 53); a L. johnsonii TeCH-tag sequence disclosed herein is MHRIM KG (LjM ccA-N7G; SEQ ID NO: 54); and a H. somni TeCH-tag sequence disclosed herein is MRGRRLG (HsM ccA-N7G; SEQ ID NO: 55).

The polypeptide fused to the TeCH-tag can be any polypeptide of interest, and is not limited to GFP as described herein.

Exemplary polypeptides to be modified include structural polypeptides, polypeptides involved in signaling, polypeptides involved in small and large molecule transport, enzymes, hormones, neuropeptides, antimicrobial peptides, growth regulators, and the like. Working examples that have already been made and tested include the M ccB/TeCH-tag system on green fluorescent protein (GFP), maltose binding protein (MBP), the catalytic domain (residues 1-321) of protein tyrosine phosphatase 1B (PTP1B₁₋₃₂₁), a recombinant anti-GFP antibody based on the Trastuzumab scaffold (a-GFP rAb), protein L, and an affibody that recognizes endothelial growth factor receptor (zEGFR).

In one aspect, a method of modifying the C-terminus of a polypeptide, comprises providing a polypeptide, wherein the polypeptide is a substrate of an E1-like superfamily enzyme; reacting the polypeptide, the E1-like superfamily enzyme, and ATP under conditions to O-A M Pylate the C-terminus of the polypeptide; and reacting the C-terminally O-A M Pylated polypeptide with a nucleophile comprising a functional group to provide a modified polypeptide comprising the C-terminal functional group.

In one aspect, the polypeptide substrate is a polypeptide fusion comprising a TeCH-tag as described above.

In one aspect, the nucleophile is a thiol, a hydrazine, an alkoxyamine, a hydroxylamine, an amine, or an alcohol.

In a more specific aspect, the nucleophile is a thiol nucleophile and the C-terminal functional group is a thioester. Exemplary thiol nucleophiles include N-acetyl-L-cysteine, N-acetylcysteamine, sodium 2-mercaptoethane sulfonate (M esna), dithiothreitol (DTT), L-cysteine (Cys), and the like.

Exemplary hydrazides that can be formed from the reaction may have the formula R-NR¹-NR²-R³, wherein R is acyl, sulfonyl, phosporyl, or phosphinyl, and R¹, R² and R³ are hydrogen or a hydrocarbon functional group. Hydrazine nucleophiles are used to produce the hydrazide products.

Exemplary oximes that can be formed from the reaction may have the formula R⁴R⁵C═NOH, wherein R⁴ is or a hydrocarbon functional group and R⁵ is hydrogen or a hydrocarbon functional group. Alkoxyamine nucleophiles are used to produce the oxime products.

Exemplary amides that can be formed from the reaction have the formula R⁶-C(═O)—NR⁷R⁸, wherein R⁶, R⁷ and R⁸ are each independently hydrogen or a hydrocarbon functional group. A mine nucleophiles are used to produce the amide products.

Advantageously, the modification to provide a C-terminal thioester provides versatile intermediates for synthesis of chemically tailored proteins. For example, the C-terminal thioester can be labeled with a cargo molecule as described in more detail below.

In one aspect, the C-terminal thioester can be transthiolated using a reducing agent such as dithiothreitol (DTT), betamercaptoethanol (BM E), 4-mercaptophenylacetic acid (M PAA), and the like.

In another aspect, the C-terminal functional group, e.g., the thioester functional group, can be reacted with a bioconjugation agent. When the C-terminal functional group that is installed is a thioester, it can undergo trans-thiolation with Cys or an N-terminal Cys peptide, followed by S-to-N acyl shift (a reaction known as native chemical ligation) to form an amide bond. The product of this reaction can react with a maleimide. Conjugation to an N-terminal Cys peptide can be used to introduce functional groups such as azides and the other functional groups listed herein. For example, when the reactive group comprises cysteine, an exemplary bioconjugation agent is malemide.

In one aspect, the bioconjugation agent can further comprise a cargo molecule.

In one aspect, the bioconjugation agent can be reacted with a reactive molecule comprising a cargo molecule to provide a fusion polypeptide labeled with the cargo molecule.

Exemplary cargo molecules include an imaging agent, a pharmaceutical agent, a nanoparticle, a radiolabel, a polymer, an amino acid, and the like.

Exemplary imaging agents include fluorescent imaging agents such as organic dyes (e.g., AlexaFluor®; Thermo Fisher Scientific, Waltham, MA), quantum dots, and the like.

Exemplary pharmaceutical agents include small molecules as well as polypeptides (e.g., therapeutic peptides, antibodies, and antibody fragments), therapeutic nucleic acids (e.g., siRNA, antisense RNA), and cellular therapeutics (e.g., CAR T cells).

Exemplary nanoparticles include polymeric nanoparticles, liposomes, dendrimers, and the like. Nanoparticles can be used to deliver drugs, gene therapy, radiotherapy and photodynamic therapy agents.

Exemplary radiolabels include radiotracers labeled with carbon-11, fluorine-18, iodine-125, technetium-99m, and the like.

In a further aspect, when the C-terminal functional group is a thioester, the thioester can be exchanged with a reactive moiety comprising a N-terminal cysteine to provide a peptide bond.

Exemplary polymers include polyethylene glycols.

In another aspect, a C-terminal thioester polypeptide can be ligated to a peptide with high efficiency. Thus, in an aspect, the method further comprises reacting the C-terminal thioester-modified polypeptide with a target peptide (e.g., AFA) in the presence of a ligase (e.g., subtiligase) to provide a target peptide fused to the C-terminus of the polypeptide.

Exemplary ligases include subtiligase, a genetically engineered subtilisin protease variant that acts to biotinylate newly generated N termini.

Advantageously, installing specific amino acids such as Lys or Arg at the C-terminus of peptides can enhance their analysis by tandem mass spectrometry, for example, facilitating proteomics analysis.

The disclosure is further illustrated by the following non-limiting examples.

### EXAMPLES

Here, we report the mechanism-guided design of an M ccB-based toolbox for C-terminal activation and protein modification. We show that E. coli MccB has a latent capacity to catalyze C-terminal adenylation on non-native substrates. The resultant C-terminal peptidyl-O-AMP electrophile can react with a variety of exogenous nucleophiles, including hydrazines, alkoxyamines, amines, and thiols, to form diverse C-terminal functional groups. We develop the Thioesterification C-terminal Handle (TeCH-tag), a sequence that enables M ccB-catalyzed, ATP-driven synthesis of protein C-terminal thioesters, an important class of protein bioconjugation intermediates that were previously only directly accessible via a method that relies on engineered self-splicing inteins. By mining the natural diversity of the M ccB family, we identify two additional M ccB/TeCH-tag pairs that are mutually orthogonal to each other and to the E. coli system, as well as a more promiscuous M ccB homolog. We apply the M ccB/TeCH-tag system for high-yield, ATP-dependent protein bioconjugation via expressed protein ligation and enzyme-catalyzed expressed protein ligation. We find that M ccB/TeCH-tag is compatible with other bioconjugation enzymes such as sortase, enabling synthesis of dual N- and C-terminally functionalized protein conjugates. We also develop a more promiscuous homolog of M ccB for synthesis of Ub-derived peptide thioester substrates that can be used as substrates for the Lysine Acylation using Conjugating Enzymes (LA CE) system. These strategies mimic the chemical logic of peptide bond synthesis that is widespread in biology for high-yield in vitro synthesis of protein bioconjugates that will advance our understanding of biological systems.

### Enzyme Mechanism-Guided Design of a Tool for C-Terminal Functionalization

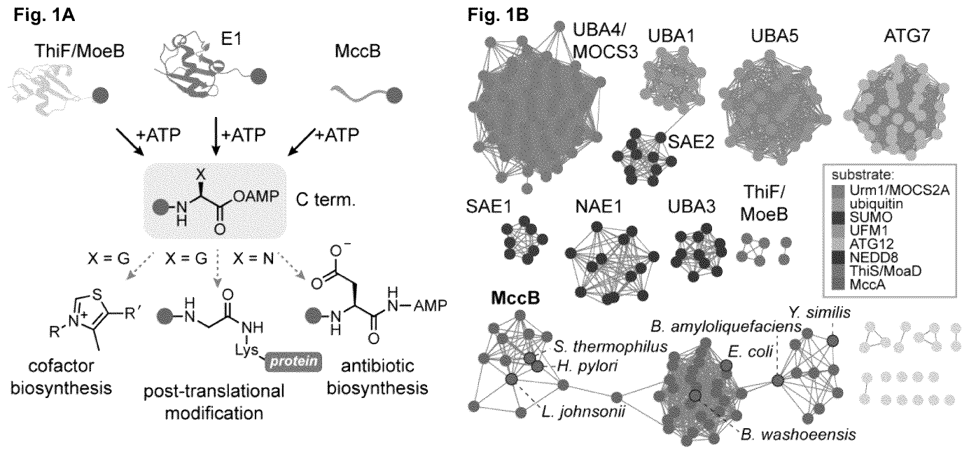

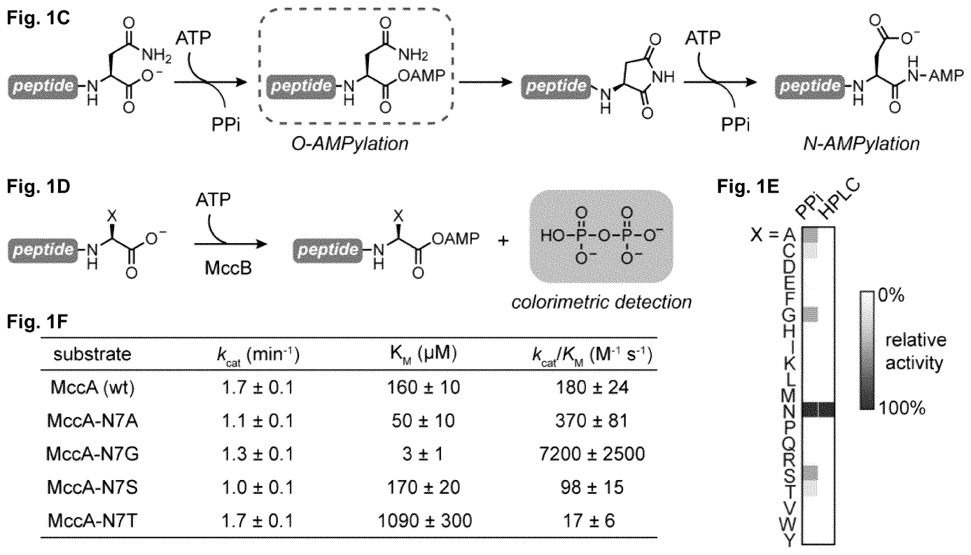

E. coli MccB is a member of the E1-like enzyme superfamily that catalyzes conversion of the C-terminal Asn residue of its heptapeptide substrate, M ccA (MRTGNAN; SEQ ID NO: 18), to an isoasparagine (isoAsn)-AMP phosphoramidate (FIGS. 1A and 1B). As its first mechanistic step, M ccB is proposed to catalyze C-terminal adenylation (or O-A M Pylation) of M ccA, producing an MccA-O-AMP intermediate that is captured by the B-carboxamido nitrogen group of the C-terminal Asn (N7) residue to form a succinimide intermediate (FIG. 1C). This succinimide is subsequently N-A M Pylated and hydrolyzed to form the C-terminal isoA sn-A M P product. This modified heptapeptide is further tailored by phosphoramidate aminopropylation and acts as a ‘Trojan horse’ antibiotic that is cleaved by an endogenous protease in target cells to form isoAsn-AMP, an aspartyl-adenylate mimic that inhibits the aspartyl-tRNA synthetase. While most families within the E1-like superfamily act on ubiquitin-like β-grasp fold proteins with a C-terminal Gly residue, M ccB family enzymes are unique in that they accept as their substrates short, genetically encoded peptides terminating with C-terminal Asn (FIGS. 1A and 1B). We hypothesized that if we substituted the C-terminal Asn of the M ccA substrate with another amino acid, MccB would retain the ability to O-AM Pylate the non-native substrate and that in the absence of a cis nucleophile in the substrate, the peptidyl-O-AM P electrophile could react with an exogenous nucleophile for C-terminal functionalization.

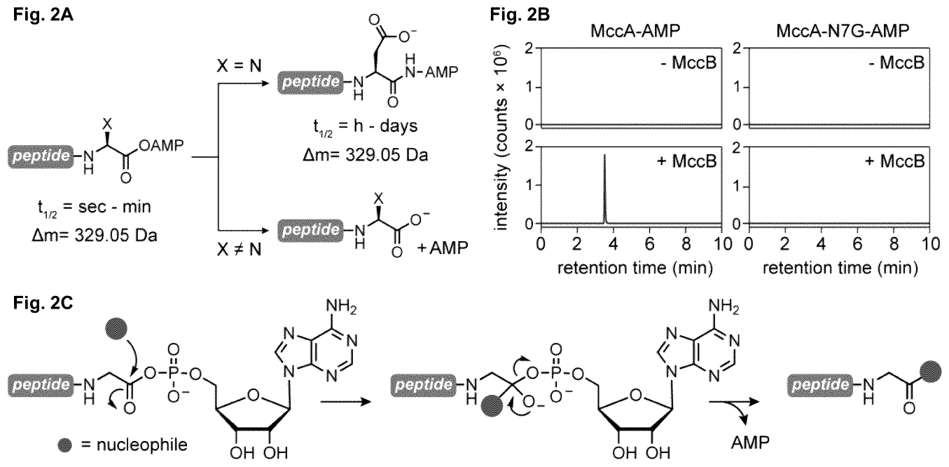

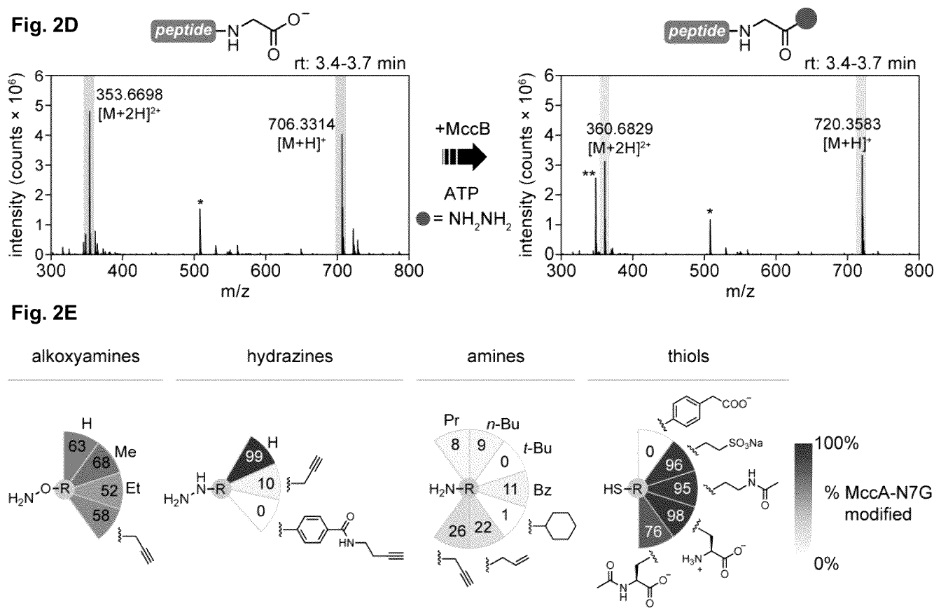

To test the hypothesis that M ccB retains a latent capacity to O-AM Pylate peptide substrates lacking the conserved C-terminal Asn residue, we synthesized a small library of non-native M ccA peptides in which the C-terminal residue was varied to the 19 amino acids other than Asn (M ccA-N7X). Because the peptidyl-O-AMP product of M ccB-catalyzed O-AM Pylation is expected to be hydrolytically unstable, we initially screened the ability of these MccA variants to stimulate ATP consumption by MccB using an enzyme-coupled assay to detect formation of the pyrophosphate (PPi) by-product (FIG. 1D). We observed substantial stimulation of PPi formation when M ccA-N7A, N7G, N7S, or N 7T were used as substrates for M ccB (FIG. 1E). We determined the steady-state kinetic parameters for M ccB-catalyzed pyrophosphate formation with each of these substrates and found that the catalytic efficiencies (Kcal/KM) were similar to or exceeded the K_{cat}/KM measured for wild-type M ccA (FIG. 1F). We next tested whether this ATP consumption resulted in the formation of any stably AM Pylated product using an HPLC-MS assay. While we observed a clear+329.0485 Da peak corresponding to A M Pylation when wild-type M ccA was used as a substrate, no stably A M Pylated product was observed for the MccA-N7A, N7G, N7S, or N7T variants (FIGS. 2A and 2B; Table 2). These results are consistent with a previous study that showed that N7 is required for microcin C7's antimicrobial activity (Kazakov et al. “Amino acid residues required for maturation, cell uptake, and processing of translation inhibitor microcin.” C. J. Bacteriol. 189, 2114-2118, 2007). Together these data raise two possibilities: 1) Binding of M ccA-N 7X variants to M ccB stimulates unproductive ATP hydrolysis; or 2) M ccA-N 7X variants bind to M ccB and are O-AM Pylated, but the resulting mixed anhydride is hydrolytically unstable and cannot be detected by HPLC-M S.

TABLE 2
LC-TOF MS characterization of MccA-N7X variants
and potential O-AMPylation modifications.

| | reactant (unmodified peptide) | | | O-AMPylated product | | |
|---|---|---|---|---|---|---|
| MccA- | calc. | obs. | obs. | calc. | obs. | obs. |
| N7X | M + H⁺ | M + H⁺ | M + 2H⁺ | M + H⁺ | M + H⁺ | M + 2H⁺ |
| N7A | 720.3458 | 720.3481 | 360.6791 | 1049.3988 | n.d. | n.d. |
| N7C | 752.3178 | 752.3152 | 376.6573 | 1081.3708 | n.d. | n.d. |
| N7D | 764.3356 | 764.3369 | 382.6749 | 1093.3886 | n.d. | n.d. |
| N7E | 778.3512 | 778.3523 | 389.6834 | 1107.4042 | n.d. | n.d. |
| N7F | 796.3771 | 796.3776 | 398.6974 | 1125.4301 | n.d. | n.d. |
| N7G | 706.3301 | 706.3359 | 353.6784 | 1035.3831 | n.d. | n.d. |
| N7H | 786.3676 | 786.3685 | 393.6918 | 1115.4206 | n.d. | n.d. |
| N7I | 762.3927 | 762.3933 | 381.7039 | 1091.4457 | n.d. | n.d. |
| N7K | 777.4036 | 777.4050 | 389.2102 | 1106.4566 | n.d. | n.d. |
| N7L | 762.3927 | 762.3964 | 381.7145 | 1091.4457 | n.d. | n.d. |
| N7M | 780.3491 | 780.3497 | 390.6805 | 1109.4021 | n.d. | n.d. |
| N7N | 763.3516 | 763.3561 | 382.7822 | 1092.4046 | 1092.4046 | 546.7062 |
| N7P | 746.3614 | 746.3628 | 373.6895 | 1075.4144 | n.d. | n.d. |
| N7Q | 777.3672 | 777.3677 | 389.1878 | 1106.4202 | n.d. | n.d. |
| N7R | 804.4098 | 805.4104 | 403.2140 | 1134.4628 | n.d. | n.d. |
| N7S | 736.3407 | 736.3440 | 368.6821 | 1065.3937 | n.d. | n.d. |
| N7T | 750.3563 | 750.3570 | 375.6847 | 1079.4093 | n.d. | n.d. |
| N7V | 748.3771 | 748.3781 | 374.6964 | 1077.4301 | n.d. | n.d. |
| N7W | 835.3880 | 835.3876 | 418.2000 | 1164.4410 | n.d. | n.d. |
| N7Y | 812.3720 | 812.3721 | 406.6917 | 1141.4250 | n.d. | n.d. |

n.d., not detected.

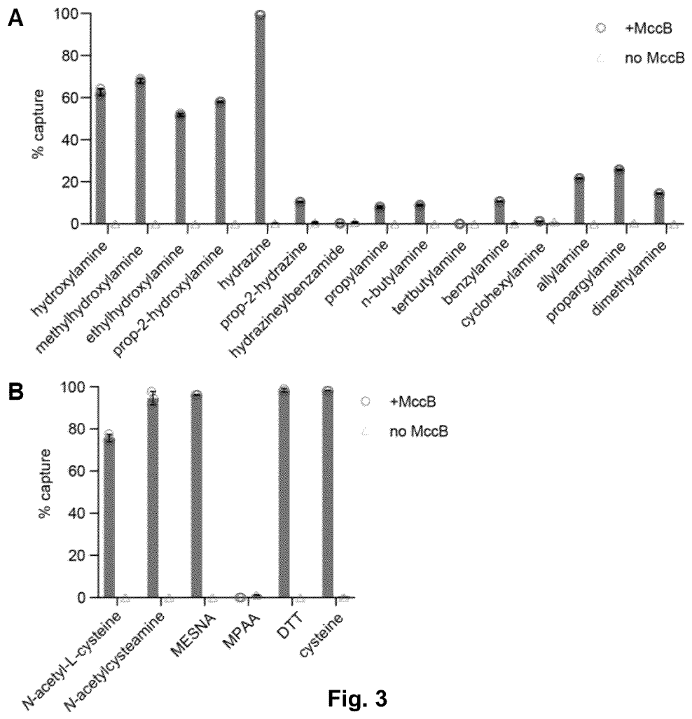

We hypothesized that if an electrophilically activated O-A M Pylated C terminus is formed, an exogenously added nucleophile, hydrazine, could compete with water to attack the electrophilic group, resulting in M ccA-N 7X bearing a C-terminal hydrazide that could be distinguished from the unmodified peptide based on its mass (+14.0269 Da) (FIG. 2C). Such experiments have been used previously to detect electrophilic enzymatic intermediates including anhydrides and thioesters (Saha et al. “M ultimodal activation of the ubiquitin ligase SCF by Nedd8 conjugation.” M ol. Cell 32, 21-31, 2008; Weeks et al. “Catalytic control of enzymatic fluorine specificity.” Proc. Natl. Acad. Sci. 109, 19667-19672, 2012). To test this hypothesis, we included 150 mM hydrazine in reactions containing M ccB, ATP, and MccA-N 7G (M+H⁺=706.3306). HPLC-M S analysis of these reactions revealed formation of a new peak corresponding to formation of a C-terminal hydrazide (M+H⁺=720.3583) that was dependent on M ccB, ATP, and hydrazine (FIG. 2D). Similar results were obtained using hydroxylamine as the trapping nucleophile (FIG. 2D, FIG. 3). These data support the hypothesis that M ccB catalyzes O-A M Pylation of non-A sn C-terminal residues and provide direct evidence for the O-A M Pylated intermediate in the proposed mechanism of M ccB.

We next sought to define features of the nucleophile that impact the efficiency of capture of the O-AM Pylated intermediate. We initially screened a panel of hydroxylamines, hydrazines, and amines by measuring their ability to modify the C terminus of M ccA-N7G in an M ccB-dependent manner using HPLC-MS (FIG. 2E, FIG. 3, Table 3). Among hydroxylamine nucleophiles, increasing the size of the O-substituent decreased the efficiency of C-terminal modification. Similarly, we found that a recently reported phenylhydrazine-based nucleophilic probe (N-(but-3-yn-1-yl)-4-(2-hydrazineylethyl)benzamide) was unable to modify the C terminus of M ccA-N 7G. These results suggest that nucleophile size is an important factor in the efficiency of M ccB-catalyzed M ccA-N 7G modification and raise the hypothesis that the intermediate is not released from the enzyme but reacts with exogenous nucleophiles within the enzyme active site. In contrast to the efficient capture observed with hydroxylamine and hydrazine, small primary and secondary amines modified MccA inefficiently (0-26%) (FIG. 2E, FIG. 3). These results suggest that nucleophile strength is also an important factor in efficient capture of the O-A M Pylated intermediate.

TABLE 3
Chemical reagents used for nucleophilic capture of MccB-activated C-termini.

| Reagent | CAS No. | Supplier |
|---|---|---|
| hydroxylamine hydrochloride | 5470-11-1 | Sigma-Aldrich |
| O-methylhydroxylamine hydrochloride | 593-56-6 | Sigma-Aldrich |
| O-ethylhydroxylamine hydrochloride | 3332-29-4 | Sigma-Aldrich |
| O-(prop-2-yn-1yl)hydroxylamine | 21663-79-6 | Ambeed |
| hydrochloride | | |
| hydrazine monohydrate | 7803-57-8 | Sigma-Aldrich |
| prop-2-yn-1-ylhydrazine hydrochloride | 1187368-95-1 | Enamine |
| N-(but-3-yn-1-yl)-4-hydrazinebenzamide | 2081100-00-5 | Sigma-Aldrich |
| propylamine hydrochloride | 556-53-6 | Sigma-Aldrich |
| butylamine | 109-73-9 | Sigma-Aldrich |
| tert-butylamine hydrochloride | 10017-37-5 | Sigma-Aldrich |
| benzylamine hydrochloride | 3287-99-8 | Sigma-Aldrich |
| cyclohexylamine | 108-91-8 | Sigma-Aldrich |
| allylamine hydrochloride | 10017-11-5 | Fisher Scientific |
| propargylamine | 2450-71-7 | AK Scientific |
| dimethylamine hydrochloride | 506-59-2 | Sigma-Aldrich |
| N-acetyl-L-cysteine | 616-91-1 | Sigma-Aldrich |
| N-acetylcysteamine | 1190-73-4 | Sigma-Aldrich |
| dithiothreitol | 27565-41-9 | Gold Biotechnology |
| sodium 2-mercaptoethanesulfonate (Mesna) | 19767-45-4 | Sigma-Aldrich |
| 4-mercaptophenylacetic acid | 39161-84-7 | Fisher Scientific |
| L-cysteine | 52-90-4 | Sigma-Aldrich |

We next tested whether MccB could catalyze ATP-dependent C-terminal thioesterification of M ccA-N 7G in the presence of thiol nucleophiles. We tested a panel of thiols comprised of N-acetyl-L-cysteine, N-aceylcysteamine, DTT, sodium 2-mercaptothane sulfonate (M esna), and 4-mercaptophenylacetic acid (M PAA). With the exception of M PAA, we found that all of these thiol nucleophiles modified M ccA-N 7G in 76-98% yield (FIG. 2E, FIG. 3). We attribute the inability of M PAA to modify M ccA-N7G to the constraints on nucleophile size and structure that are imposed by the enzyme active site. These results demonstrate that the M ccA-N 7G-O-AM P intermediate can be efficiently transferred to thiol nucleophiles for C-terminal thioesterification.

### Applying M ccB for Protein C-Terminal Thioesterification and Peptide Bond Formation

Protein and peptide C-terminal thioesters are key reactive species in both biological and chemical peptide bond formation pathways. For example, in the canonical ubiquitination cascade, an E1 catalyzes C-terminal O-AM Pylation of ubiquitin (Ub), activating Ub for nucleophilic attack by a Cys side chain to generate an E1-bound Ub C-terminal thioester (denoted E1˜Ub). E1˜Ub then undergoes transthioesterification with a Cys side chain of an E2 ubiquitin-conjugating enzyme to form E2˜Ub. Finally, an E3 ubiquitin ligase binds both E2˜Ub and the substrate to catalyze S-to-N acyl transfer to a Lys side chain from the substrate to form an isopeptide bond. Similarly, in native chemical ligation (NCL), a peptide C-terminal thioester undergoes transthioesterification with a peptide bearing an N-terminal Cys residue, follow by S-to-N acyl shift to form a native peptide bond. While the initial transthioesterification step in NCL is reversible, the S-to-N acyl shift that forms the amide bond is irreversible, driving the reaction to high yield.

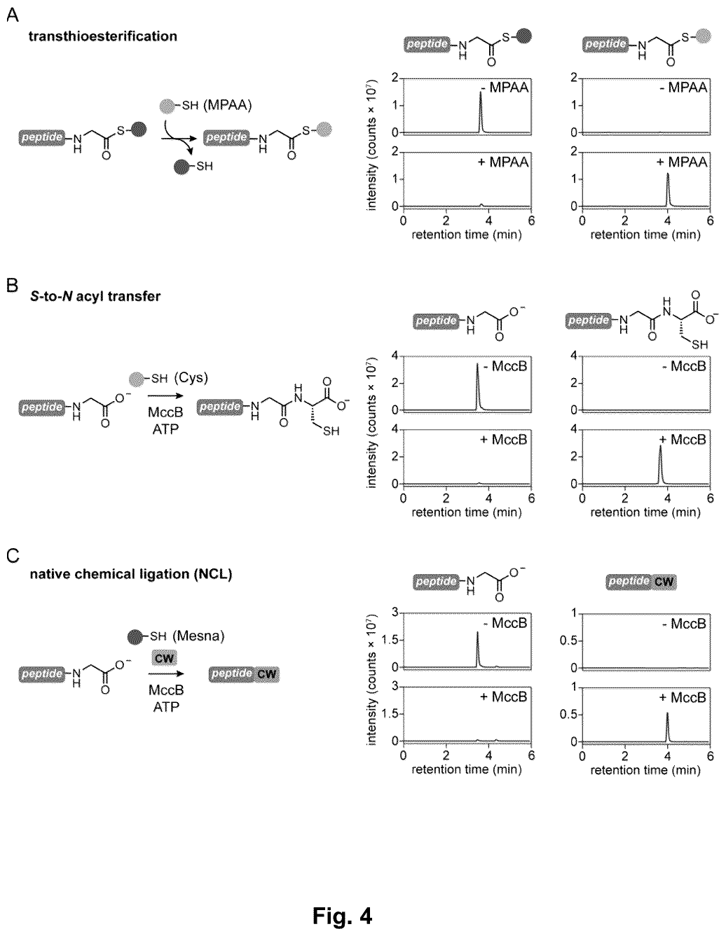

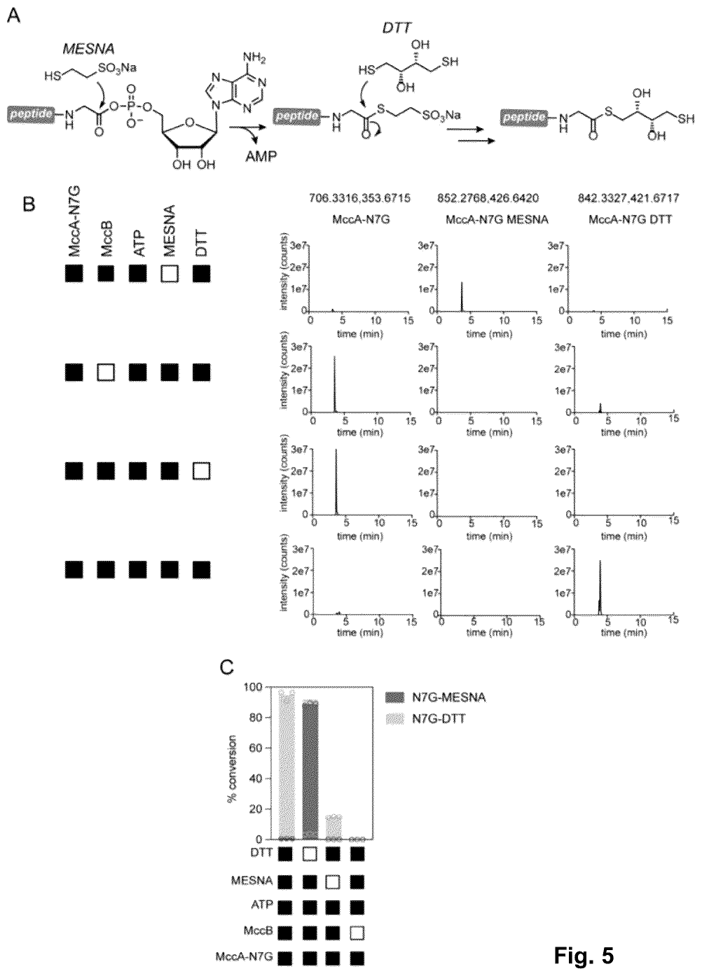

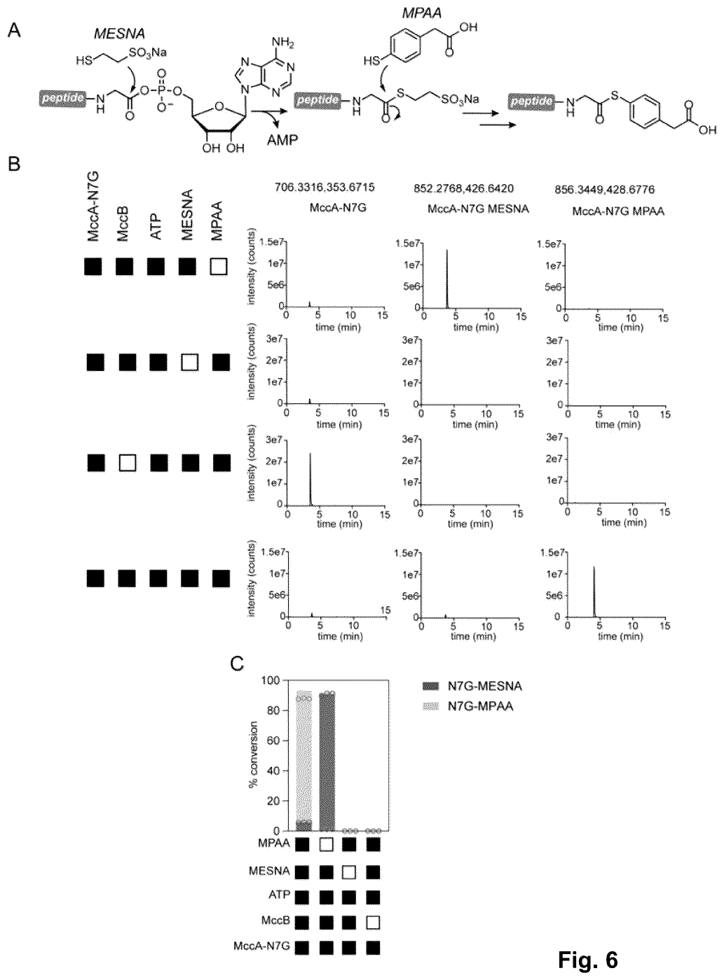

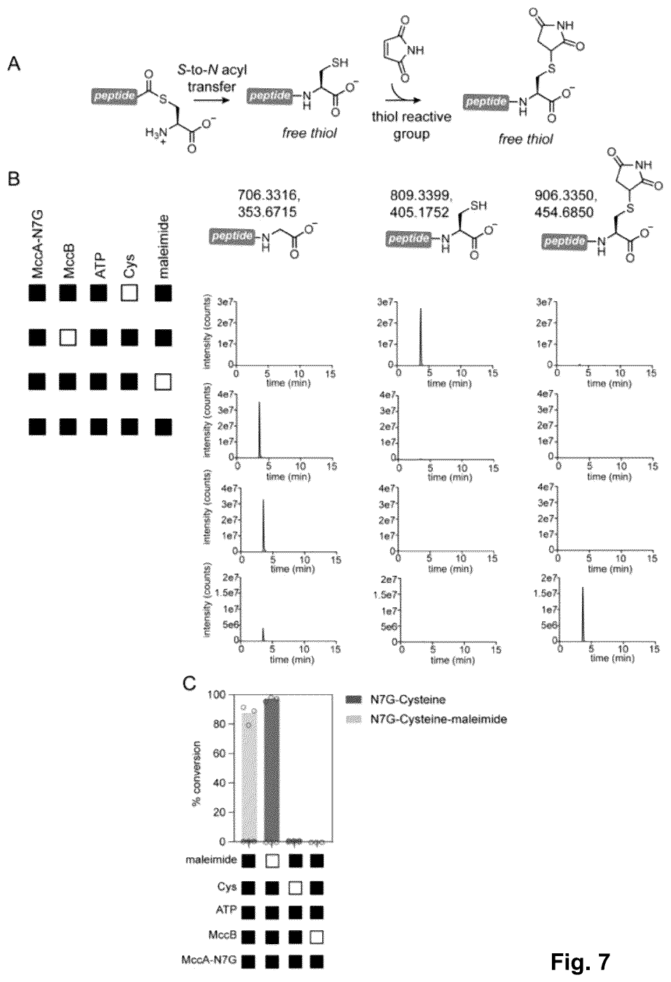

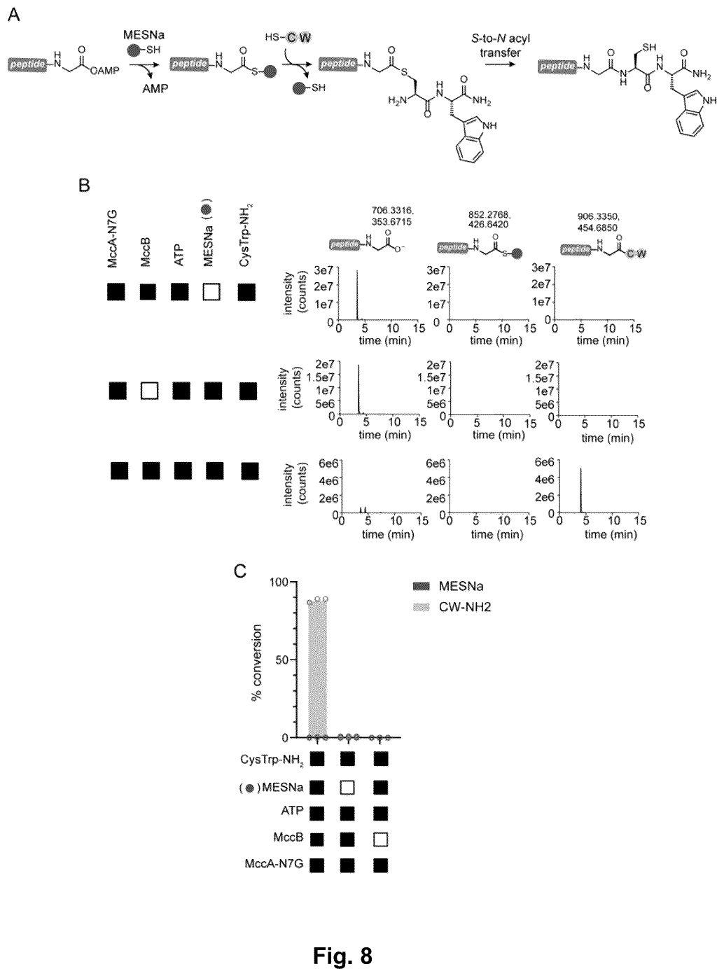

We found that C-terminal thioesters synthesized by M ccB could undergo both transthioesterification with M PAA, a thiol that cannot efficiently capture the M ccA-N 7G-O-AMP electrophile directly (FIGS. 4-6). This result suggests that transthioesterification occurs outside the enzyme active site and is not subject to the same steric restraints as the initial thioesterification reaction. We also found that when L-cysteine is used as the thiol donor, the thioester underwent S-to-N acyl shift to form a peptide bond (FIG. 4, FIG. 7). Encouraged by these results, we sought to test whether N-terminal Cys peptides could similarly capture M ccA-N7G-O-AMP and undergo S-to-N acyl shift to enable a peptide ligation reaction. We first tested a Cys-Trp dipeptide nucleophile and observed no modification of MccA-N7G, suggesting that the larger nucleophile is unable to access MccA-N7G-O-AMP within the M ccB active site (FIG. 8). However, when we included both Mesna and Cys-Trp in the reaction, M ccA-N 7G was converted to M ccA-N 7G-CW in 82% yield in 4 hours (FIG. 4, FIG. 8). This result suggests that M ccA-N 7G can undergo M ccB-catalyzed thioesterification with Mesna, followed by transthioesterification and S-to-N acyl shift with Cys-Trp in one pot, enabling enzyme-catalyzed NCL starting from an unactivated peptide with a C-terminal carboxylate.

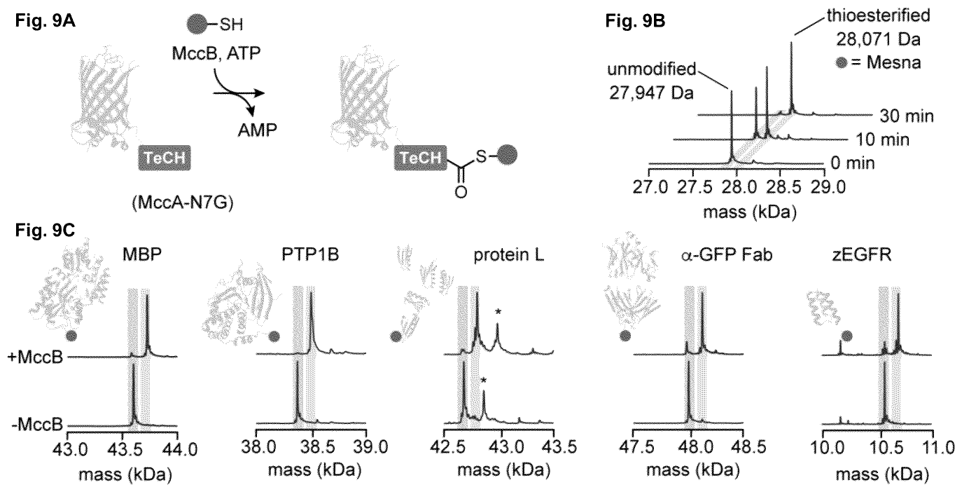

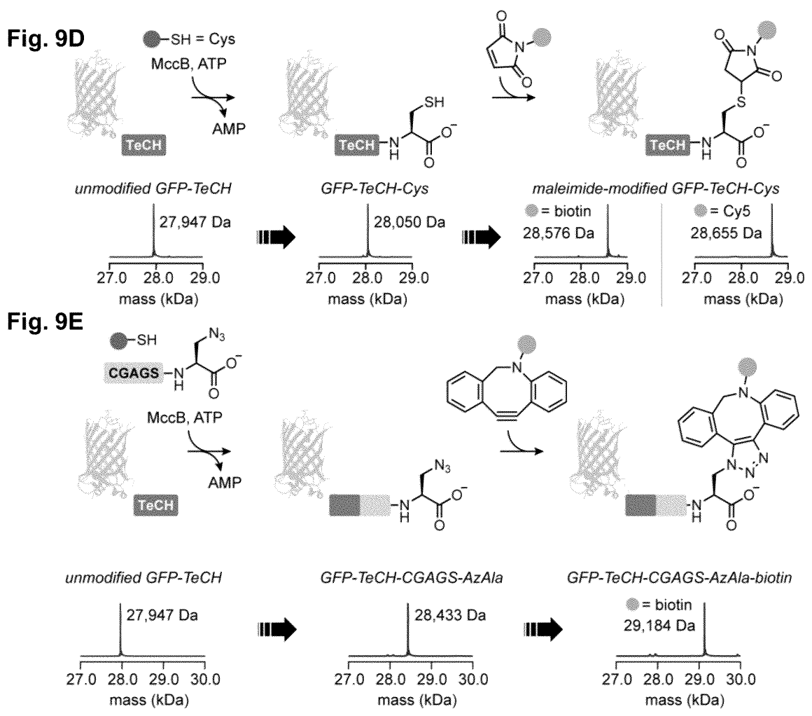

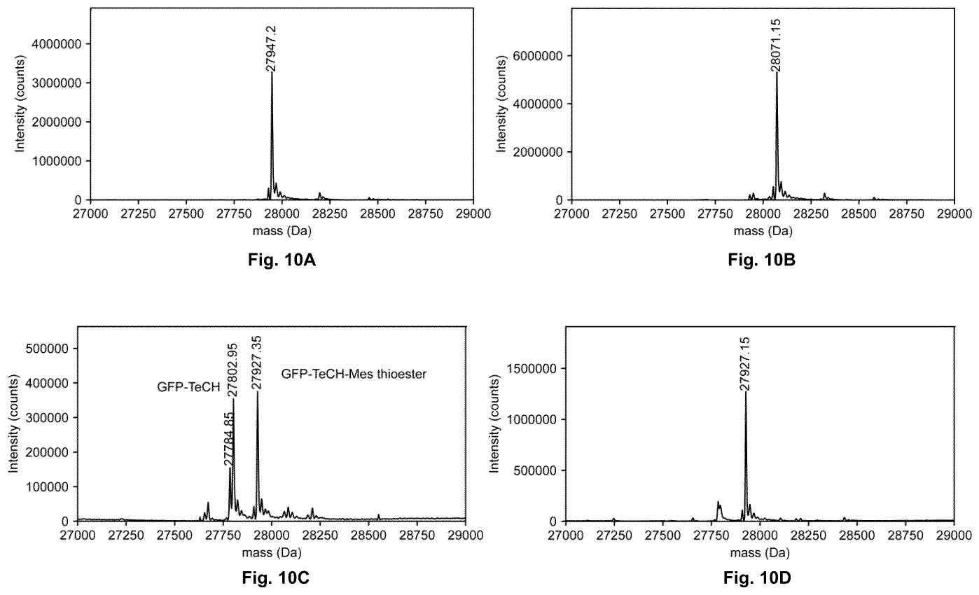

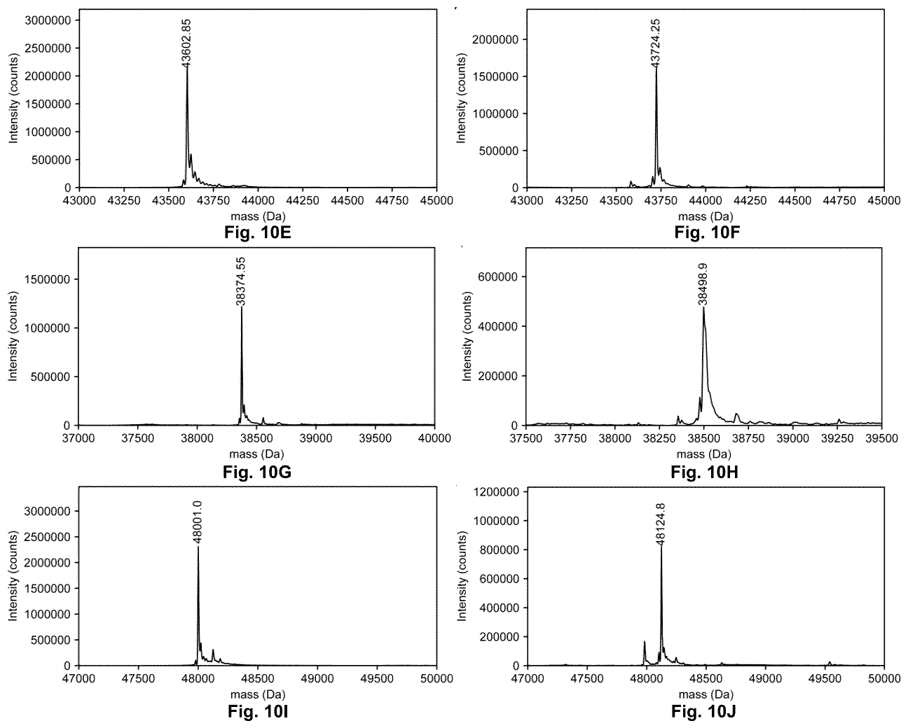

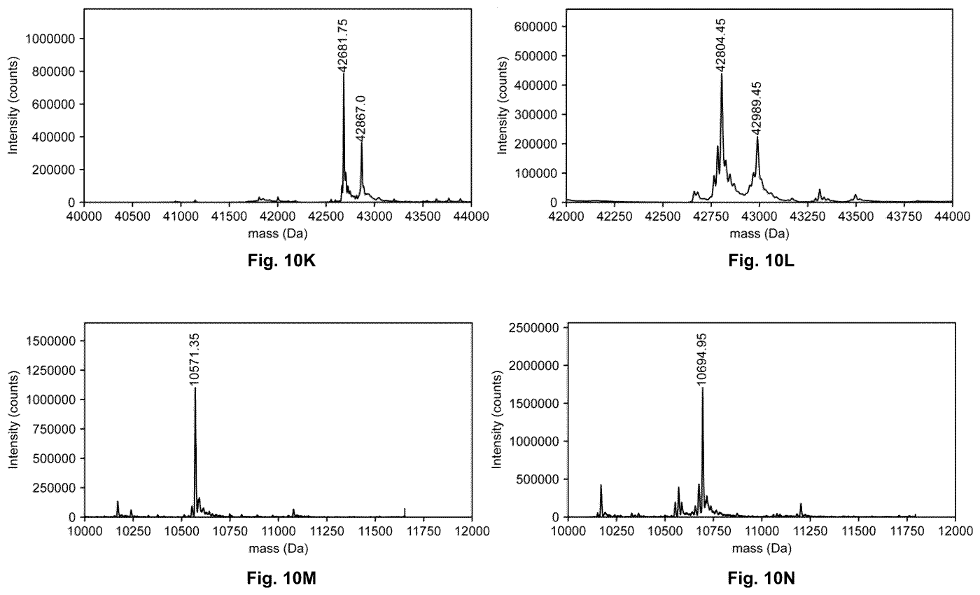

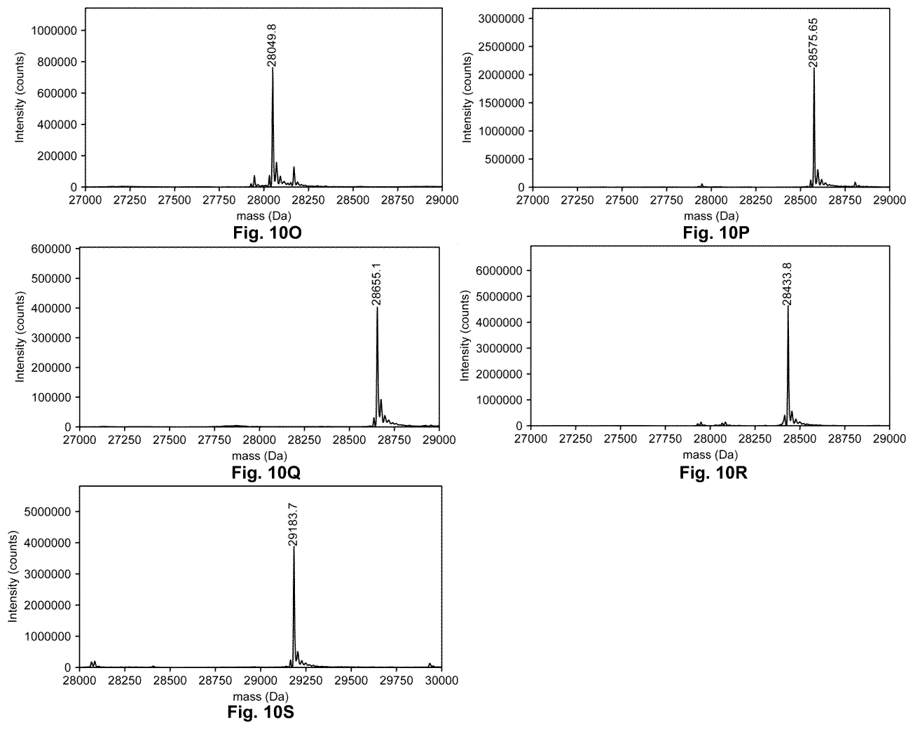

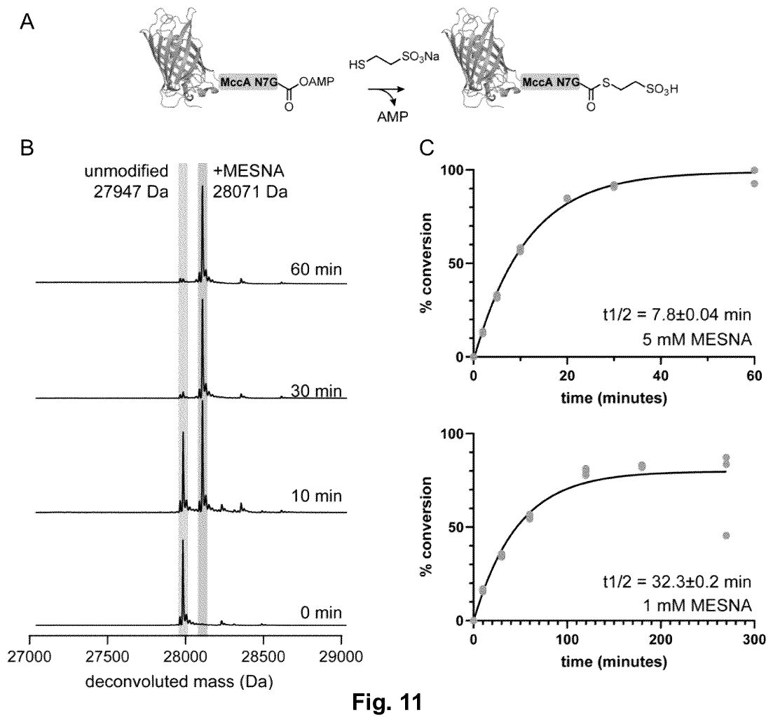

C-terminal protein thioesters are key intermediates in expressed protein ligation (EPL), a powerful protein semisynthesis method that involves NCL between a recombinantly expressed protein C-terminal thioester and a synthetic N-terminal Cys peptide to form a native peptide bond. Prior applications of EPL have relied on fusion of the protein to be modified with an engineered intein to generate the protein C-terminal thioester. Encouraged by the ability of M ccB to catalyze M ccA-N 7G thioesterification to activate it for C-terminal peptide ligation, we hypothesized that M ccA-N7G could be developed as a tag for C-terminal protein thioesterification. Previous functional characterization of M ccB demonstrated that an N-terminal maltose binding protein (MBP) fusion of M ccA is recognized as a substrate by M ccB and can be modified with an N—P bond to A M P (Bantysh et al. “Enzymatic synthesis of bioinformatically predicted microcin C-like compounds encoded by diverse bacteria.” mBio 5, e01059-14, 2014). To test whether M ccA-N 7G could similarly enable protein C-terminal thioesterification, we fused GFP to M ccA-N 7G (a sequence that we term the Thioesterification C-terminal Handle, or TeCH-tag) (FIG. 9A, FIG. 10A). In the presence of 5 mM M esna and 5 mM ATP, we found that M ccB catalyzed near-quantitative conversion of GFP-TeCH-tag to the M esna thioester with 30 min (FIG. 9B, FIG. 10B, FIG. 11). In comparison, a GFP-TeCH-M xe GyrA intein fusion protein yielded a mixture of GFP-TeCH-Mes (40%) and hydrolyzed GFP-TeCH (60%) (FIG. 10C). When the partially hydrolyzed intein-generated thioester was treated with M ccB, ATP, and Mesna, it was converted nearly quantitatively to the M es thioester (FIG. 10D). The M ccB/TeCH-tag system therefore provides an efficient method to generate C-terminal protein thioesters.

To examine the general utility of the M ccB/TeCH-tag system, we appended the TeCH-tag to a diverse panel of proteins, including MBP; the catalytic domain (residues 1-321) of protein-tyrosine phosphatase 1B (PT P1B 1-321); an anti-GFP recombinant antibody (a-GFP rAb); protein L; and an endothelial growth factor receptor (EGFR)-targeting affibody (zEGFR). We treated these TeCH-tag fusion proteins (50 mM) with M ccB (5 mM), ATP (5 mM), and Mesna (5 mM) and found that they were thioesterified in near-quantitative yield within 1-16 hours (FIG. 9C, FIG. 10E-10N). While GFP, MBP, and PTP 1B 1-321 were completely converted to thioester in 1 h, zEGFR required 6 h, and protein L and a-GFP rAb required 16 h. Notably, the a-GFP rAb, based on a scaffold derived from the therapeutic antibody Trastuzumab, contains five disulfide bonds linking its light and heavy chains. We found that the rAb could be thioesterified in quantitative yield under conditions that omit reducing agents other than Mesna, keeping the disulfide bonds required for antibody function intact. These results demonstrate that the M ccB/TeCH-tag system is broadly applicable for generating protein C-terminal thioesters.

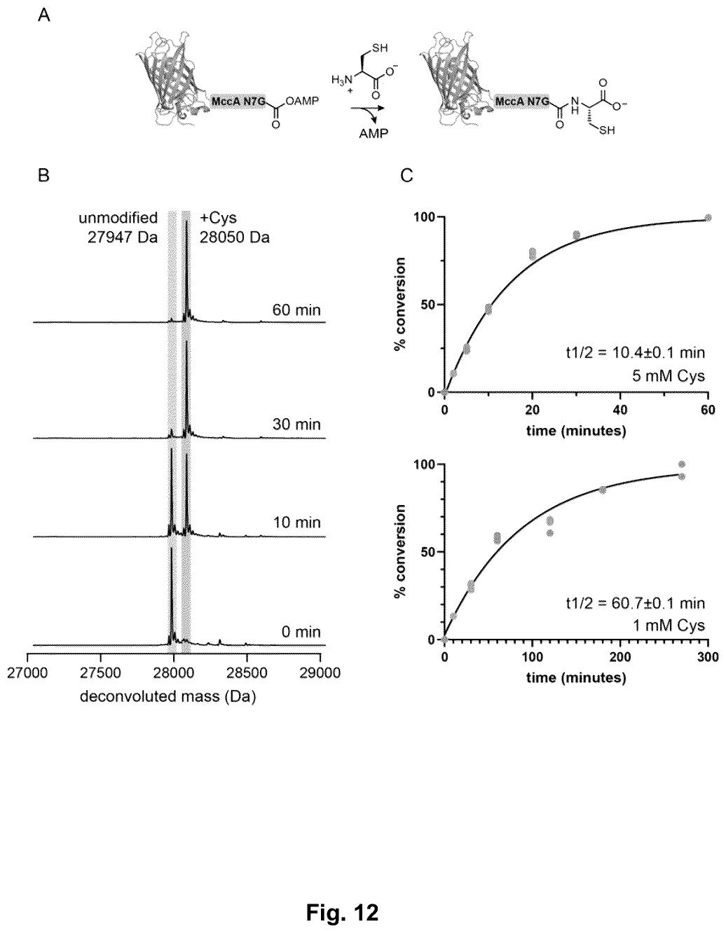

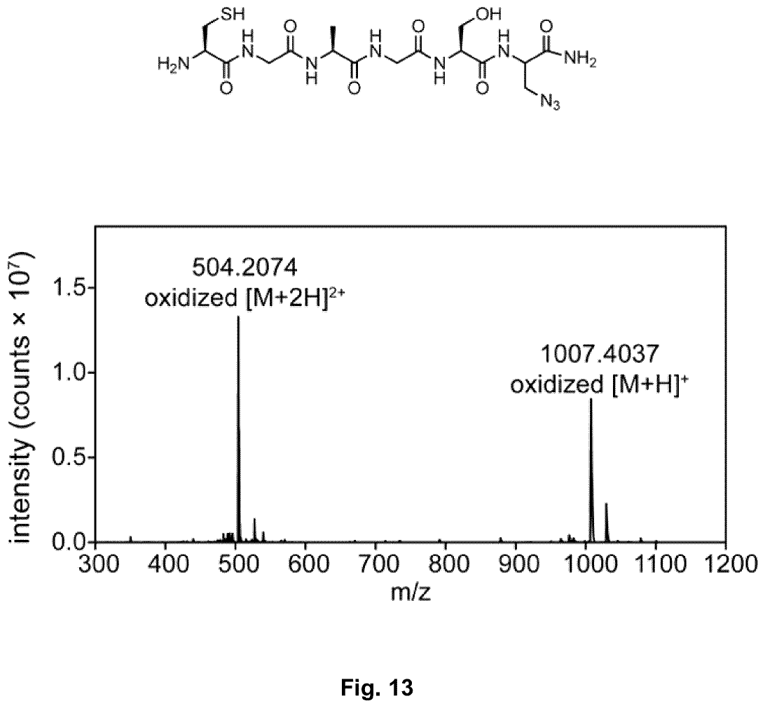

We next examined whether the M ccB/TeCH-tag system could be deployed to expand the toolbox for C-terminal thioester formation in the context of EPL. We initially tested whether M ccB could catalyze incorporation of single Cys residue at the C terminus of a Cys-free variant of GFP (cfGFP) via an amide bond. We incubated cfGFP (50 mM) with MccB (5 mM), ATP (5 mM), and Cys (5 mM) and found that Cys was ligated to cfGFP in in 99% yield in 1 h (FIG. 9D, FIGS. 10A and 10O, FIG. 12). Following Cys incorporation, the Cys side chain was quantitatively modified with biotin-maleimide or Cy5-maleimide, demonstrating that S-to-N acyl shift had occurred to produce a free Cys side chain (FIG. 9D, FIGS. 10P and 10Q). We tested whether a workflow involving thioesterification with M esna, transthioesterification with an N-terminal Cys peptide, and S-to-N acyl shift could be applied for ligation of cfGFP to a synthetic N-terminal Cys peptide bearing an azide functional group. We performed a reaction in which TeCH-tagged cfGFP was incubated with M ccB, ATP, Mesna, and peptide with the sequence CGAGS-3-azido-I-Ala (SEQ ID NO: 56) (Table 4, FIG. 13). We found that cfGFP-TeCH-tag could be quantitatively modified with the azide-bearing peptide (FIG. 9E, FIG. 10R). The azide modified protein could then undergo strain-promoted azide-alkyne cycloaddition (SPA AC) with dibenzocyclooctyne (DBCO)-biotin (FIG. 9E, FIG. 10S). M ccB can thus serve as a catalyst for protein C-terminal thioesterification to enable EPL.

TABLE 4
LC-TOF MS characterization of peptides
used in bioconjugation experiments.

| peptide | calc. M + H⁺ | obs. M + H⁺ |
|---|---|---|
| CGAGSazidoA (SEQ ID NO: 56) | 505.1703 | 505.2023 |
| AFAGAGSazidoK (C-term. amide) | 732.3667 | 733.3845 |
| (SEQ ID NO: 57) | | |

Natural Sequence Diversity Encompasses Orthogonal M ccA/M ccB Pairs

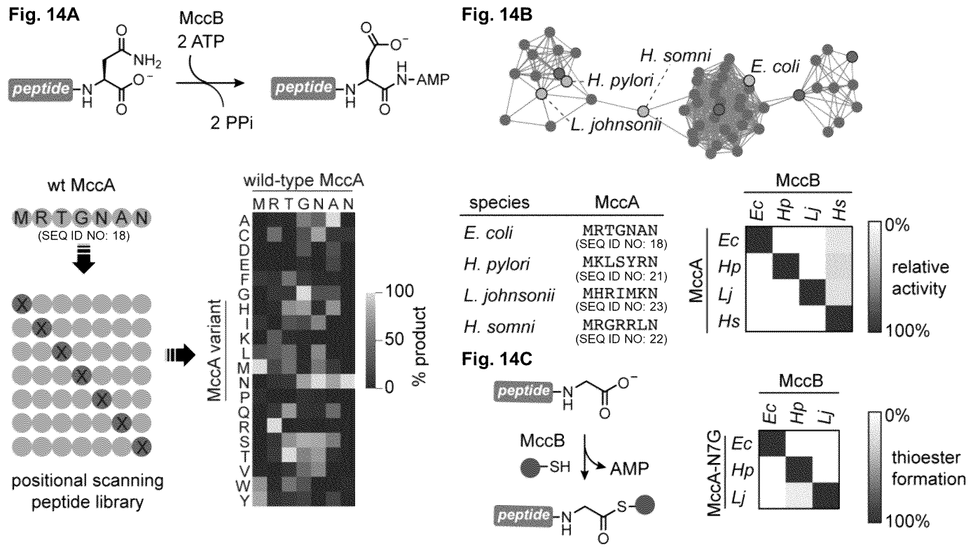

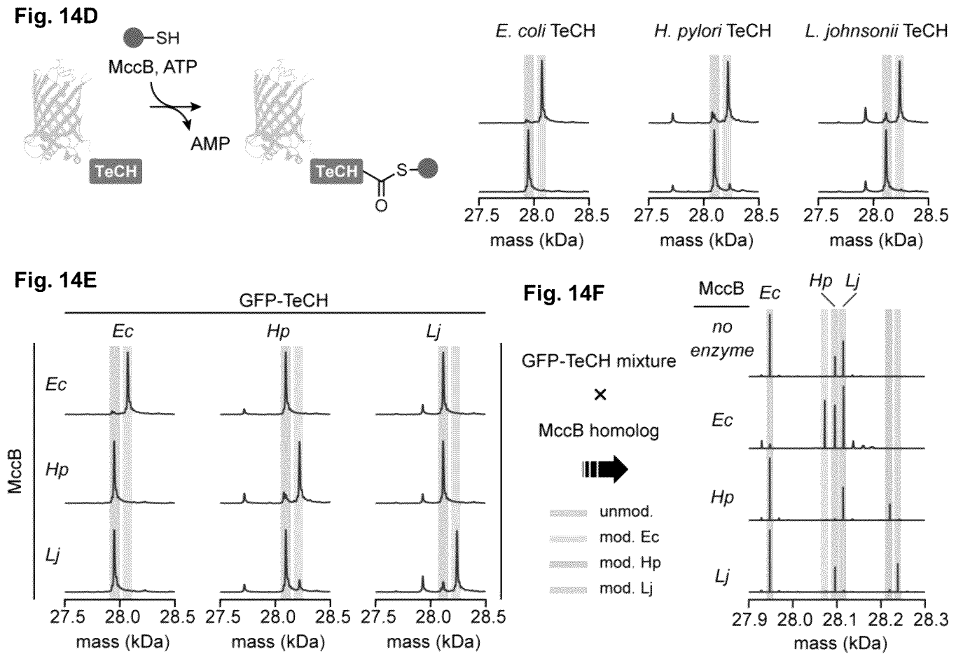

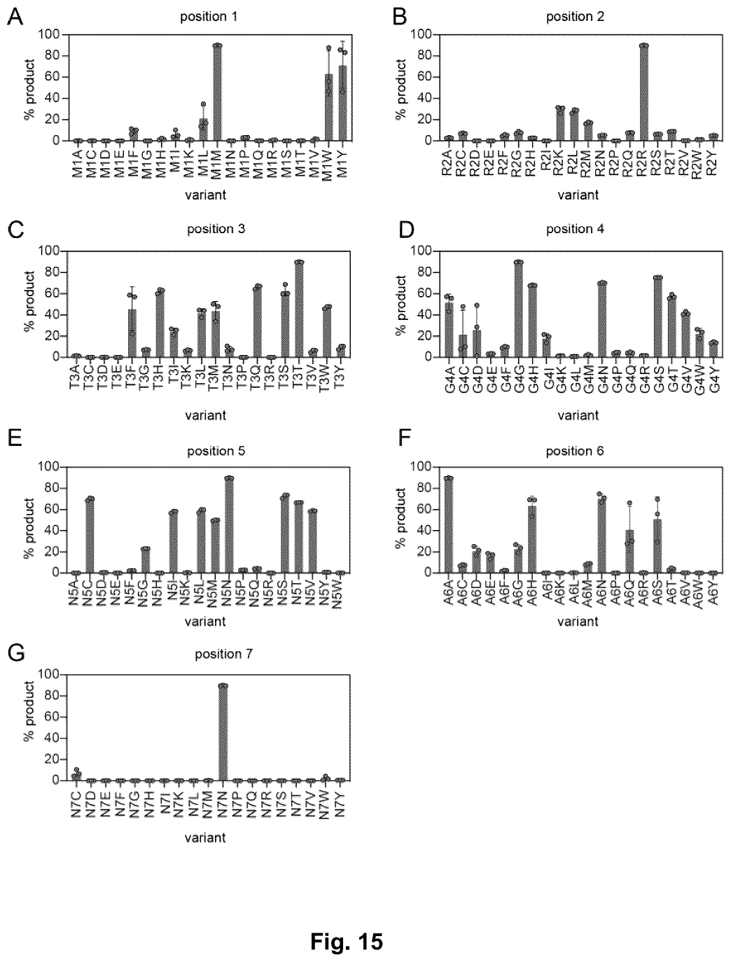

To examine the sequence specificity of MccB for the MccA substrate, we synthesized a library of peptides in which each of the amino acid in each position of M ccA was varied to the 19 non-native canonical amino acids (FIG. 14A, FIG. 15, Table 5). We then measured formation of the C-terminally N-AM Pylated phosphoramidate product for each of the MccA variants to query the stringency of MccB's sequence specificity. We found that only wild-type M ccA was fully converted to product over 16 h. Consistent with our previous results, no product formation was observed if the seventh position was varied to an amino acid other than Asn. Outside the C-terminal residue, substitutions in the first two positions of M ccA had the largest effect on M ccB activity, with significant product formation observed only for the M 1W, M 1Y, R²K, and R²L variants. At positions 3-6, significant product formation was observed for 6-8 different substitutions in each position. However, none of these variants were converted to product in quantitative yield despite the long reaction time, suggesting that M ccB is an epitope-specific enzyme. Steady-state kinetics analysis of Ala variant peptides revealed that MccB's lower activity on MccAs with substitutions in positions 1-3 is mainly attributable to decreases in k_{cat} (FIG. 16). Our data are broadly consistent with a previous study that examined the effect of substituting M ccA positions 2-7 on microcin C 7 production and antimicrobial activity in vivo and found that positions 4-6 are most tolerant of substitutions (Bantysh et al. “Enzymatic synthesis of bioinformatically predicted microcin C-like compounds encoded by diverse bacteria.” mBio 5, e01059-14, 2014).

TABLE 5
LC-TOF MS characterization of MccA positional scanning
peptide library and N-AM Pylated products.

| | reactant (unmodified MccA | | | |
|---|---|---|---|---|
| MccA | variant) | | N-AM Pylated product | |
| variant | calc. M + H⁺ | obs. M + H⁺ | calc. M + H⁺ | obs. M + H⁺ |
| WT | 763.3516 | 763.3519 | 1092.4041 | 1092.4028 |
| M1A | 703.3482 | 703.3503 | 1032.4007 | n.d. |
| M1C | 735.3203 | 734.3119 | 1064.3728 | n.d. |
| M1D | 747.3380 | 747.3400 | 1076.3905 | n.d. |
| M1E | 761.3537 | 761.3547 | 1090.4062 | n.d. |
| M1F | 779.3795 | 779.3839 | 1108.4320 | 1108.4342 |
| M1G | 689.3326 | 689.3329 | 1018.3851 | n.d. |
| M1H | 769.3700 | 769.3710 | 1098.4225 | n.d. |
| M1I | 745.3952 | 745.4087 | 1074.4477 | 1074.4472 |
| M1K | 760.4060 | 760.4070 | 1089.4585 | n.d. |
| M1L | 745.3952 | 745.4140 | 1074.4477 | 1074.4461 |
| M1N | 746.3540 | 746.3551 | 1075.4065 | n.d. |
| M1P | 729.3639 | 729.3651 | 1058.4164 | 1058.4164 |
| M1Q | 760.3697 | 760.3694 | 1089.4222 | n.d. |
| M1R | 788.4122 | 788.4141 | 1117.4647 | n.d. |
| M1S | 719.3431 | 719.3451 | 1048.3956 | n.d. |
| M1T | 733.3666 | 733.3596 | 1062.4191 | n.d. |
| M1V | 731.3795 | 731.3794 | 1060.4320 | 1060.4322 |
| M1W | 818.3904 | 818.3946 | 1147.4429 | 1147.4443 |
| M1Y | 795.3744 | 795.3749 | 1124.4269 | 1124.4266 |
| R2A | 678.2876 | 678.3183 | 1007.3401 | 1007.3405 |
| R2C | 710.2596 | 709.2529 | 1039.3121 | 1038.3050 |
| R2D | 722.2774 | 722.2791 | 1051.3299 | n.d. |
| R2E | 736.2931 | 736.2989 | 1065.3456 | n.d. |
| R2F | 754.3189 | 754.3199 | 1083.3714 | 1083.3699 |
| R2G | 664.2719 | 664.2738 | 993.3244 | 993.3233 |
| R2H | 744.3094 | 744.3097 | 1073.3619 | 1073.3602 |
| R2I | 720.3345 | 721.3197 | 1049.3870 | n.d. |
| R2K | 735.3454 | 735.3459 | 1064.3979 | 1064.3981 |
| R2L | 720.3345 | 720.3423 | 1049.3870 | 1049.3874 |
| R2M | 738.2909 | 738.2924 | 1067.3434 | 1067.3428 |
| R2N | 721.2934 | 721.2944 | 1050.3459 | 1050.3451 |
| R2P | 704.3032 | 704.3050 | 1033.3557 | n.d. |
| R2Q | 735.3090 | 735.3115 | 1064.3615 | 1064.3608 |
| R2S | 694.2825 | 694.2860 | 1023.3350 | 1023.3348 |
| R2T | 708.2981 | 708.3002 | 1037.3506 | 1037.3504 |
| R2V | 706.3189 | 706.3214 | 1035.3714 | 1035.3673 |
| R2W | 793.3298 | 793.3316 | 1122.3823 | 1122.3804 |
| R2Y | 770.3138 | 770.3170 | 1099.3663 | 1099.3668 |
| T3A | 733.3410 | 733.3445 | 1062.3935 | n.d. |
| T3C | 765.3131 | 765.3043 | 1094.3656 | n.d. |
| T3D | 777.3308 | 777.3316 | 1106.3833 | n.d. |
| T3E | 791.3465 | 791.3478 | 1120.3990 | n.d. |
| T3F | 809.3723 | 809.3763 | 1138.4248 | 1138.4262 |
| T3G | 719.3254 | 719.3267 | 1048.3779 | 1048.3761 |
| T3H | 799.3628 | 799.3634 | 1128.4153 | 1128.4146 |
| T3I | 775.3880 | 775.3900 | 1104.4405 | 1104.4416 |
| T3K | 790.3989 | 790.4001 | 1119.4514 | n.d. |
| T3L | 775.3880 | 775.3911 | 1104.4405 | 1104.4421 |
| T3M | 793.3444 | 793.3481 | 1122.3969 | 1122.3979 |
| T3N | 776.3468 | 776.3491 | 1105.3993 | 1105.3981 |
| T3P | 759.3567 | 759.3583 | 1088.4092 | n.d. |
| T3Q | 790.3625 | 790.3638 | 1119.4150 | 1119.4150 |
| T3R | 818.4050 | 818.4054 | 1147.4575 | n.d. |
| T3S | 749.3359 | 749.3374 | 1078.3884 | 1078.3886 |
| T3V | 761.3723 | 761.3722 | 1090.4248 | 1090.4229 |
| T3W | 848.3832 | 848.3841 | 1177.4357 | 1177.4349 |
| T3Y | 825.3672 | 825.3693 | 1154.4197 | 1154.4178 |
| G4A | 777.3672 | 777.3824 | 1106.4197 | 1106.4211 |
| G4C | 809.3393 | 808.3318 | 1138.3918 | 1137.3830 |
| G4D | 821.3571 | 821.3586 | 1150.4096 | 1148.3758 |
| G4E | 835.3727 | 835.3762 | 1164.4252 | 1164.4229 |
| G4F | 853.3985 | 853.3992 | 1182.4510 | 1182.4492 |
| G4H | 843.3890 | 843.3895 | 1172.4415 | 1172.4386 |
| G4I | 819.4142 | 819.4151 | 1148.4667 | 1148.4654 |
| G4K | 834.4251 | 834.4271 | 1163.4776 | n.d. |
| G4L | 819.4142 | 819.4185 | 1148.4667 | n.d. |
| G4M | 837.3706 | 837.3711 | 1166.4231 | n.d. |
| G4N | 820.3730 | 820.3740 | 1149.4255 | 1149.4234 |
| G4P | 803.3829 | 803.3863 | 1132.4354 | 1132.4349 |
| G4Q | 834.3887 | 834.3912 | 1163.4412 | 1163.4369 |
| G4R | 862.4312 | 862.4318 | 1191.4837 | n.d. |
| G4S | 793.3621 | 793.3635 | 1122.4146 | 1122.4148 |
| G4T | 807.3778 | 807.3805 | 1136.4303 | 1136.4305 |
| G4V | 805.3985 | 805.4070 | 1134.4510 | 1134.4529 |
| G4W | 892.4094 | 892.4103 | 1221.4619 | 1221.4611 |
| G4Y | 869.3934 | 869.3931 | 1198.4459 | 1198.4447 |
| N5A | 720.3458 | 720.3496 | 1049.3983 | 1049.3992 |
| N5C | 752.3178 | 751.3114 | 1081.3703 | 1081.3653 |
| N5D | 764.3356 | 764.3376 | 1093.3881 | n.d. |
| N5E | 778.3512 | 778.3528 | 1107.4037 | n.d. |
| N5F | 796.3771 | 796.3826 | 1125.4296 | n.d. |
| N5G | 706.3301 | 706.3331 | 1035.3826 | 1035.3835 |
| N5H | 786.3676 | 786.3682 | 1115.4201 | 1115.4187 |
| N5I | 762.3927 | 762.3961 | 1091.4452 | 1091.4448 |
| N5K | 777.4036 | 777.4043 | 1106.4561 | n.d. |
| N5L | 762.3927 | 762.3953 | 1091.4452 | 1091.4452 |
| N5M | 780.3491 | 780.3505 | 1109.4016 | 1109.3989 |
| N5P | 746.3614 | 746.3630 | 1075.4139 | 1075.4119 |
| N5Q | 777.3672 | 777.3711 | 1106.4197 | 1106.4187 |
| N5R | 805.4098 | 805.4109 | 1134.4623 | n.d. |
| N5S | 736.3407 | 736.3418 | 1065.3932 | 1065.3921 |
| N5T | 750.3563 | 750.3586 | 1079.4088 | 1079.4082 |
| N5V | 748.3771 | 748.3783 | 1077.4296 | 1077.4290 |
| N5W | 835.3880 | 835.3885 | 1164.4405 | n.d. |
| N5Y | 812.3720 | 812.3738 | 1141.4245 | n.d. |
| A6C | 795.3236 | 795.3226 | 1124.3761 | 1124.3724 |
| A6D | 807.3414 | 807.3457 | 1136.3939 | 1136.3940 |
| A6E | 821.3571 | 821.3589 | 1150.4096 | 1150.4097 |
| A6F | 839.3829 | 839.3853 | 1168.4354 | 1168.4354 |
| A6G | 749.3359 | 749.3374 | 1078.3884 | 1078.3880 |
| A6H | 829.3734 | 829.3739 | 1158.4259 | 1158.4268 |
| A6I | 805.3985 | 805.3990 | 1134.4510 | n.d. |
| A6K | 820.4094 | 820.4095 | 1149.4619 | n.d. |
| A6L | 805.3985 | 805.3993 | 1134.4510 | 1134.4496 |
| A6M | 823.3549 | 823.3562 | 1152.4074 | 1152.4053 |
| A6N | 806.3574 | 806.3602 | 1135.4099 | 1135.4108 |
| A6P | 789.3672 | 789.3695 | 1118.4197 | n.d. |
| A6Q | 820.3730 | 820.3769 | 1149.4255 | n.d. |
| A6R | 848.4156 | 848.4140 | 1177.4681 | 1177.4640 |
| A6S | 779.3465 | 779.3495 | 1108.3990 | 1108.3996 |
| A6T | 793.3621 | 793.3638 | 1122.4146 | 1122.4126 |
| A6V | 791.3829 | 791.3842 | 1120.4354 | 1120.4419 |
| A6W | 878.3938 | 878.3949 | 1207.4463 | 1207.4454 |
| A6Y | 855.3778 | 855.3787 | 1184.4303 | 1184.4289 |
| N7A | 720.3458 | 720.3485 | 1049.3983 | n.d. |
| N7C | 752.3178 | 751.3112 | 1081.3703 | n.d. |
| N7D | 764.3356 | 764.3365 | 1093.3881 | n.d. |
| N7E | 778.3512 | 778.3528 | 1107.4037 | n.d. |
| N7F | 796.3771 | 796.3780 | 1125.4296 | n.d. |
| N7G | 706.3301 | 706.3347 | 1035.3826 | n.d. |
| N7H | 786.3676 | 786.3696 | 1115.4201 | n.d. |
| N7I | 762.3927 | 762.3940 | 1091.4452 | n.d. |
| N7K | 777.4036 | 777.4033 | 1106.4561 | n.d. |
| N7L | 762.3927 | 762.4004 | 1091.4452 | n.d. |
| N7M | 780.3491 | 780.3504 | 1109.4016 | n.d. |
| WT | 763.3516 | 763.3519 | 1092.4041 | 1092.4028 |
| N7P | 746.3614 | 746.3637 | 1075.4139 | n.d. |
| N7Q | 777.3672 | 777.3728 | 1106.4197 | n.d. |
| N7R | 805.4098 | 805.4103 | 1133.4623 | n.d. |
| N7S | 736.3407 | 736.3413 | 1065.3932 | n.d. |
| N7T | 750.3563 | 750.3564 | 1079.4088 | n.d. |
| N7V | 748.3771 | 748.3779 | 1077.4296 | n.d. |
| N7W | 835.3880 | 835.3895 | 1164.4405 | n.d. |
| N7Y | 812.3720 | 812.3724 | 1141.4245 | n.d. |

n.d., not detected.

Epitope-specific bioconjugation enzymes enable modification of proteins to probe their functions, to discover inhibitors and drugs, to immobilize them for catalysis, and conjugate them to cytotoxic drugs, among many other applications. However, their application can be limited by the relatively small number of available modification epitopes that restricts their use in the synthesis of more complex bioconjugates and in orthogonally targeting multiple different proteins in a mixture. As a result, there is strong interest in identifying or engineering orthogonal enzyme/substrate pairs for protein modification. We hypothesized that the natural diversity of MccA and MccB might encompass mutually orthogonal enzyme-substrate pairs (FIG. 14B). Bioinformatic analyses have revealed that many bacterial genomes that encode M ccB homologs also encode M ccA-like peptides in the same gene cluster (Zukher et al. “Reiterative synthesis by the ribosome and recognition of the N-terminal formyl group by biosynthetic machinery contribute to evolutionary conservation of the length of antibiotic microcin C peptide precursor.” mBio 10, e00768-19, 2019; Bantysh et al. “Enzymatic synthesis of bioinformatically predicted microcin C-like compounds encoded by diverse bacteria.” mBio 5, e01059-14, 2014). These analyses annotated 31 distinct, previously unknown heptapeptide M ccA sequences as well as 14 longer putative M ccAs. Based on the stringent sequence specificity of E. coli M ccB, we hypothesized that the M ccB homologs that recognize these distinct substrate sequences might be orthogonal to E. coli MccB and to one another. To test this hypothesis, we measured the activity of M ccBs from E. coli (EcM ccB), Helicobacter pylori (HpM ccB), Lactobacillus johnsonii (LjM ccB), and Histophilus somni (HsM ccB) toward both their native and non-cognate substrates using an enzyme-coupled assay to measure PPi release (FIG. 14B). All four enzymes had the highest level of activity on their native substrates. While EcM ccB, HpM ccB, and LjM ccB had <1% activity on non-cognate sequences, HsM ccB exhibited a detectable amount of activity (18-27% of HsM ccA) on all three non-cognate substrates tested. These results suggested that EcM ccB/EcM ccA, HpM ccB/HpM ccA, and LjM ccB/LjM ccA might be useful as mutually orthogonal enzyme-substrate pairs.

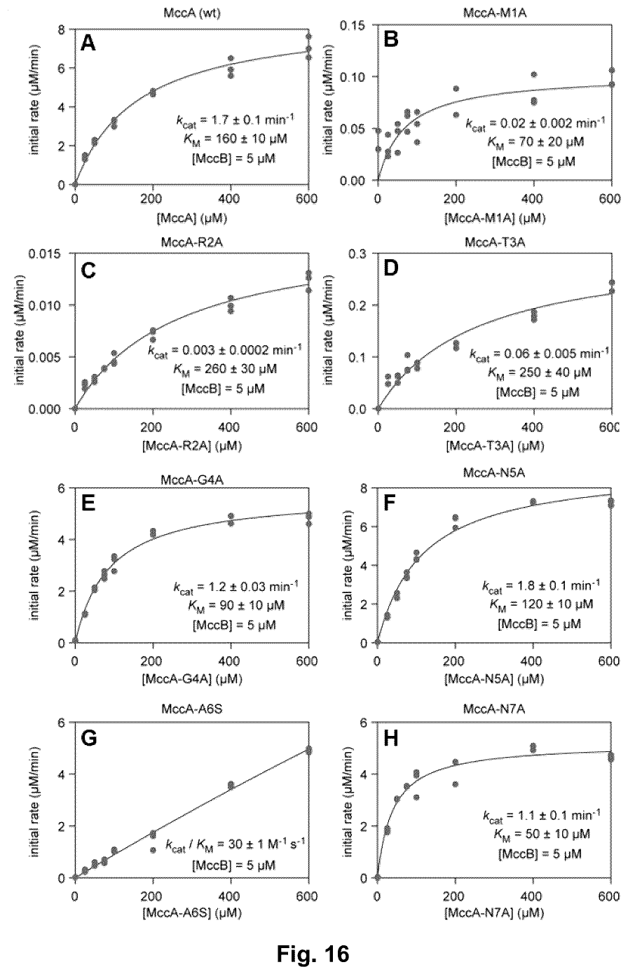

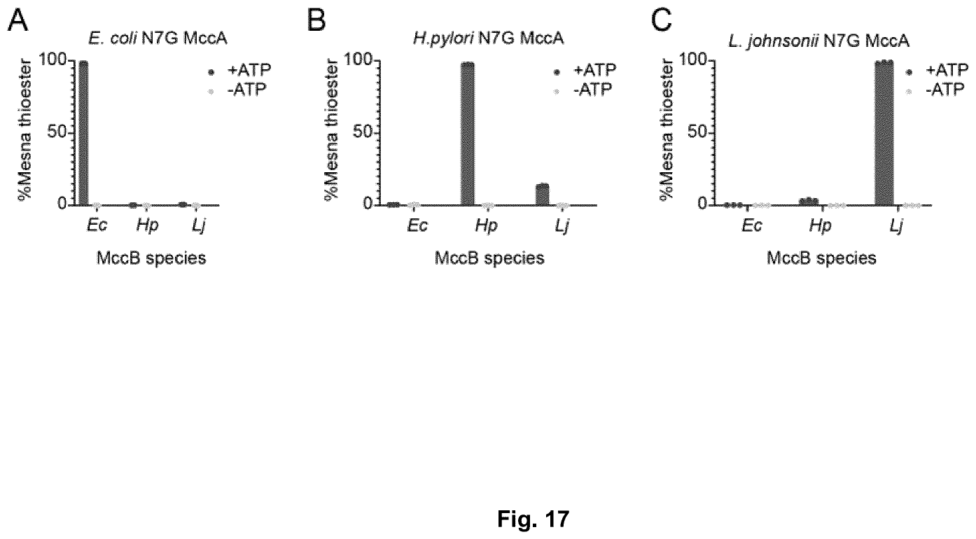

We next sought to test whether HpM ccB and LjM ccB could catalyze C-terminal thioesterification of HpM ccA-N7G and LjM ccA-N7G, respectively. We incubated each enzyme with its cognate M ccA-N 7G, ATP, and Mesna. Using LC-MS analysis, we found that each enzyme converted its cognate substrate to the C-terminal thioester in >97% yield within 16 h (FIG. 14C, FIG. 17). We next tested whether EcM ccB, HpM ccB, and LjM ccB catalyzed C-terminal thioesterification of MccA-N 7Gs from other species. We did not detect thioesterification of non-cognate substrates by the EcM ccB or LjM ccB over 16 h, while a small of amount of thioesterification (13%) of LjM ccA catalyzed by HpM ccB was observed (FIG. 14C, FIG. 17). These results suggest that these three enzyme-substrate pairs possess a high degree of mutual orthogonality in terms of their ability to catalyze C-terminal thioesterification.

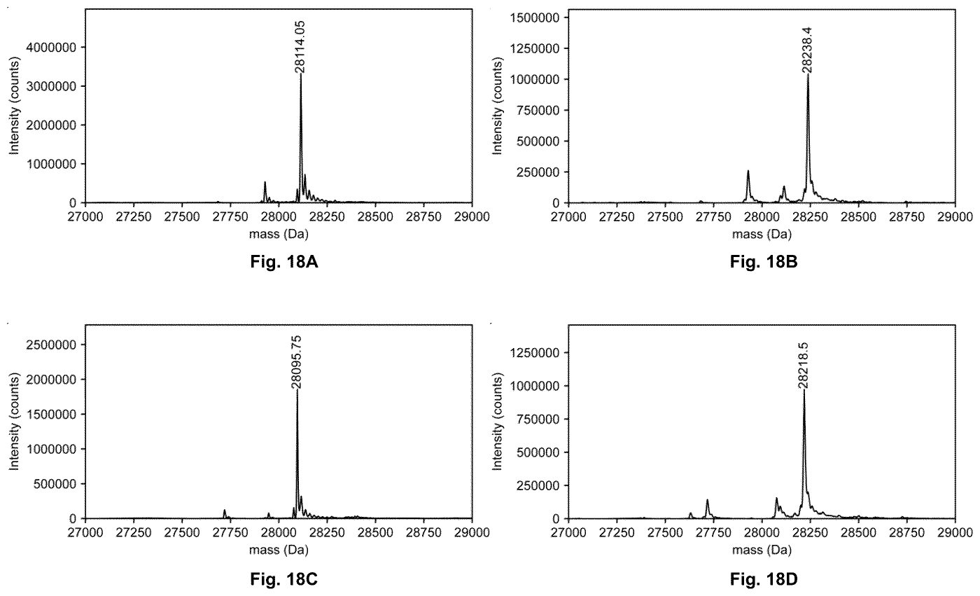

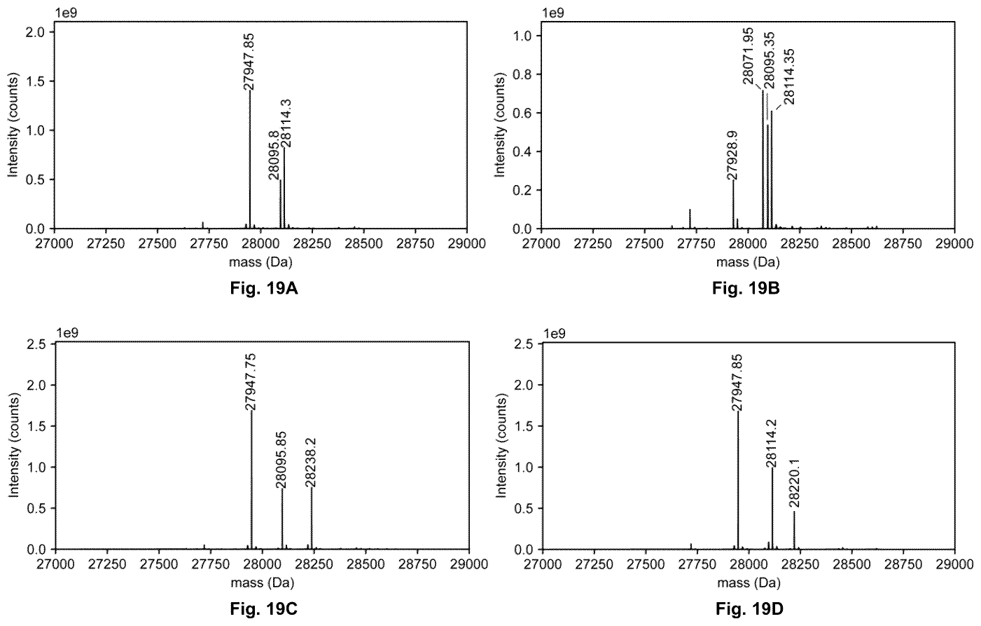

To test whether HpM ccA-N 7G and LjM ccA-N 7G could be used as TeCH-tags for C-terminal protein thioesterification, we fused these sequences to the C terminus of GFP. We found that both enzymes converted their cognate TeCH-tagged GFPs to C-terminal thioesters in the presence of 50 mM M esna (FIG. 14D, FIGS. 18A-18D). To test the orthogonality of the three M ccB/TeCH-tag systems that we developed, we examined the ability of each MccB to modify GFP-EcTeCH, GFP-HpTeCH, and GFP-LjTeCH. We found that each M ccB was only able to modify its cognate GFP-TeCH (FIG. 14E, FIGS. 18A-18D). Next, we tested whether each M ccB could selectively modify its cognate GFP-TeCH in a mixture of GFP-EcTeCH, GFP-HpTeCH, and GFP-LjTeCH (FIG. 14F, FIGS. 19A-19D). We observed that each of the M ccBs only modified its cognate substrate, even at high (50 mM) Mesna concentrations. EcM ccB/EcTeCH, HpM ccB/HpTeCH, and LjM ccB/LjTeCH therefore represent three new orthogonal C-terminal modification enzymes that greatly expand the available toolbox of epitope specific bioconjugation enzymes.

M ccB-Generated Thioesters Enhance Diverse Enzymatic Bioconjugation Strategies

Although EPL is a powerful and widely adopted tool for protein semisynthesis, enzymatic strategies for C-terminal bioconjugation offer alternative approaches with different advantages depending on the specific application and target protein. For example, compared to EPL, the enzyme-catalyzed EPL approach increases sequence flexibility at the ligation junction, while sortagging simplifies the bioconjugation strategy for applications in which sequence scars are tolerated. Based on the utility of these approaches, we sought to evaluate how the application of M ccB-generated C-terminal protein thioesters could enhance enzyme-catalyzed C-terminal bioconjugation strategies.

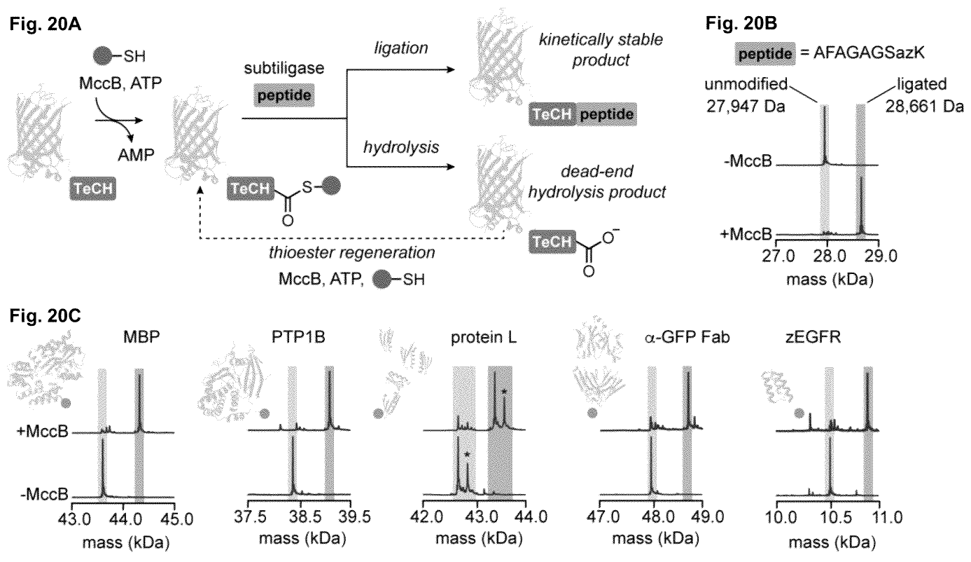

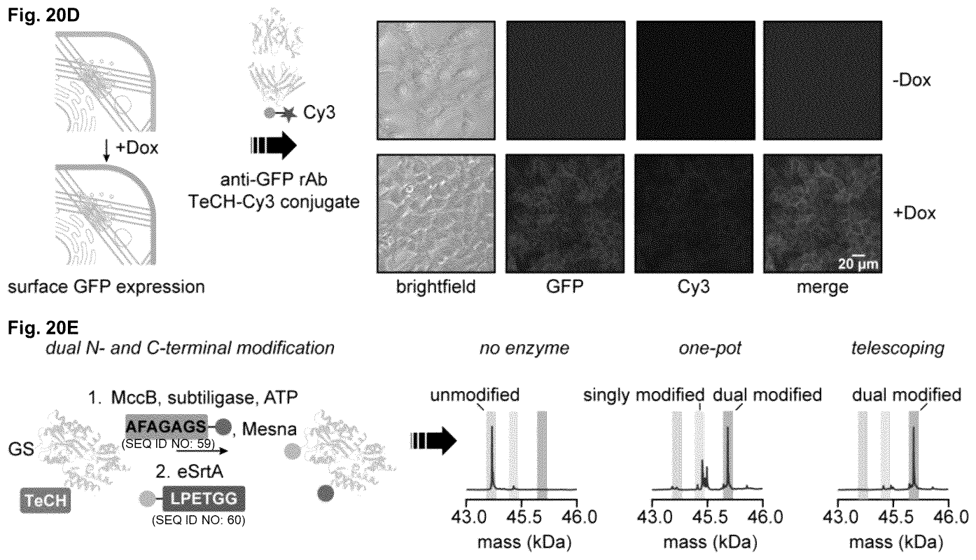

We first sought to apply M ccB in the context of enzyme-catalyzed EPL for epitope-specific bioconjugation. In the enzyme-catalyzed EPL approach, a C-terminal protein thioester is used as a substrate for the engineered peptide ligase subtiligase, which has broad N-terminal specificity and eliminates the requirement for Cys at the ligation site. While this method has been applied to study phosphoregulation of the tyrosine phosphatase PTEN by introduction of phosphoresidues at the specific sites (Henager et al. “Enzyme-catalyzed expressed protein ligation.” Nat. Methods 13, 925-927, 2016; Henager et al. “Analysis of site-specific phosphorylation of PTEN by using enzyme-catalyzed expressed protein ligation.” ChemBioChem 21, 64-68, 2020), yields were limited by a competing subtiligase-catalyzed thioester hydrolysis reaction. We hypothesized that application of M ccB for ATP-dependent thioester generation in this context would drive yields higher because it would enable thioester regeneration from the inactivated hydrolysis product (FIG. 20A).

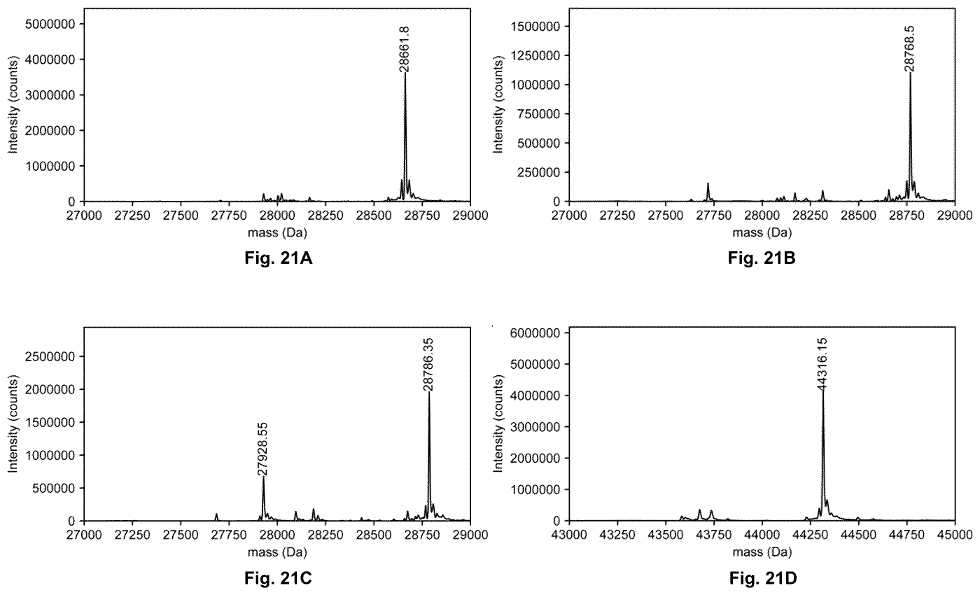

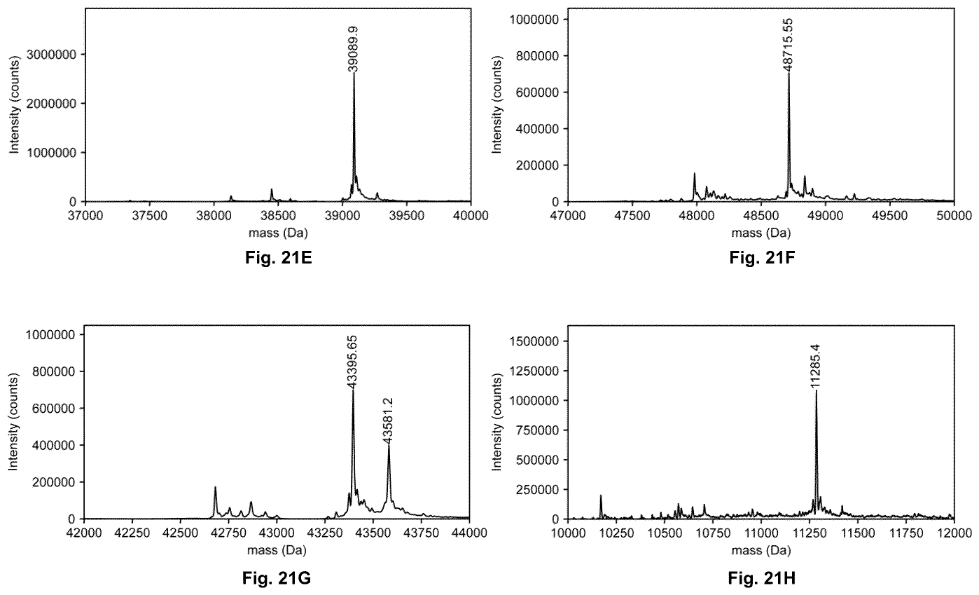

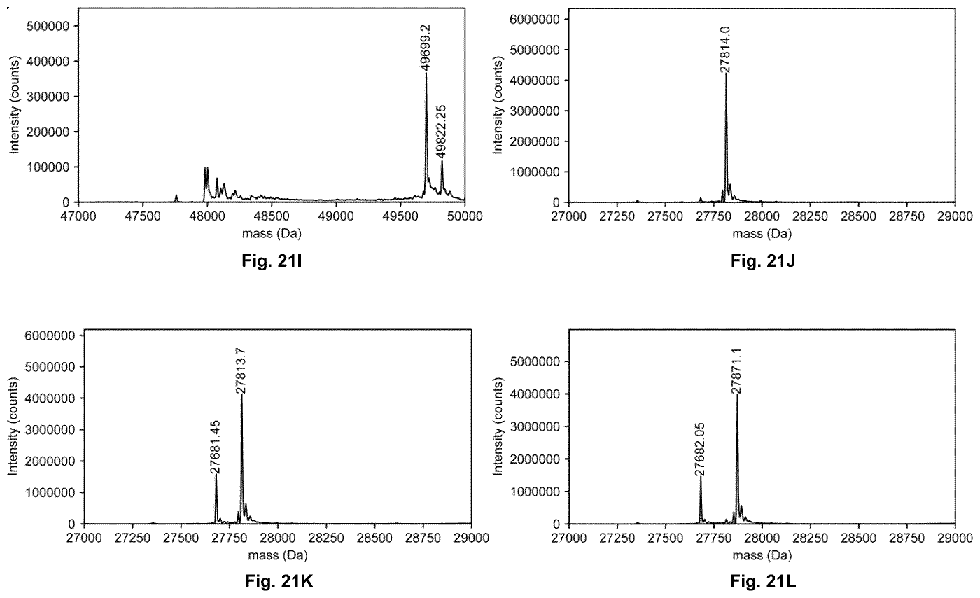

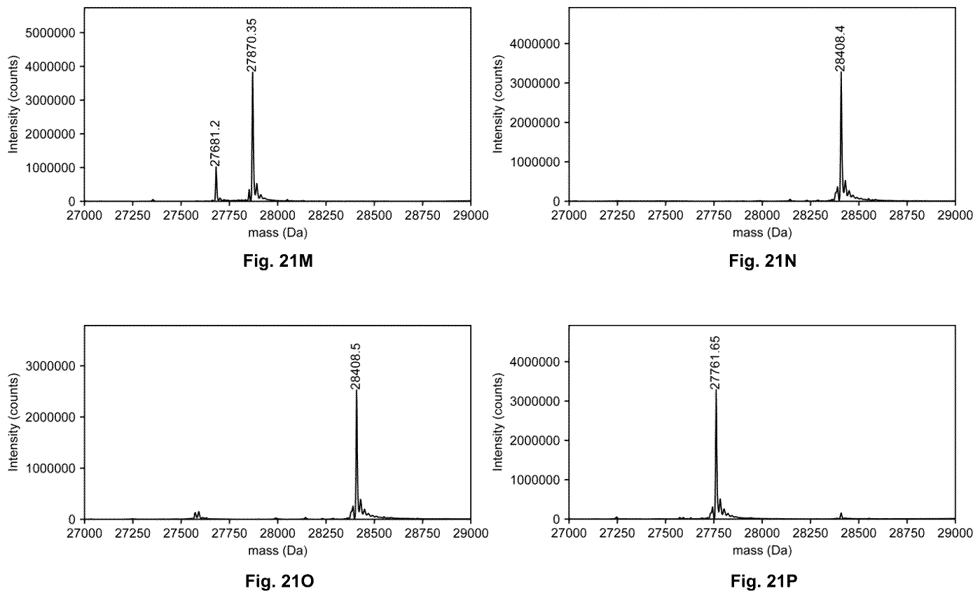

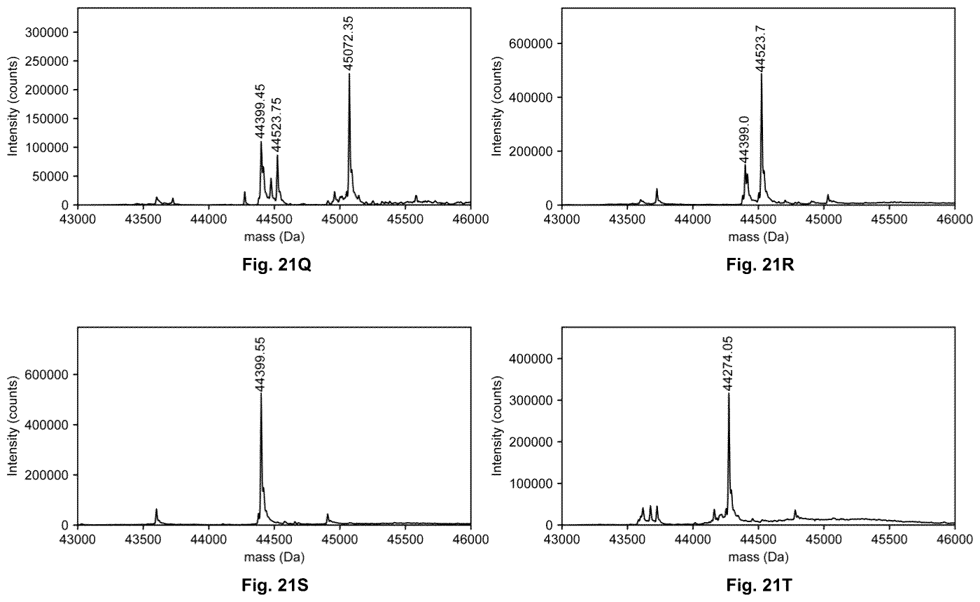

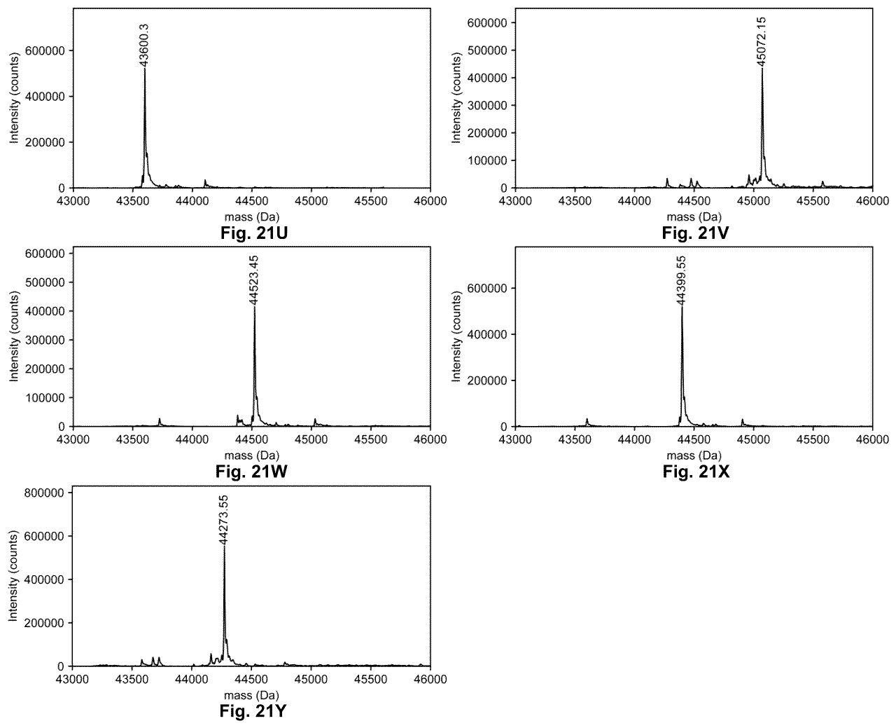

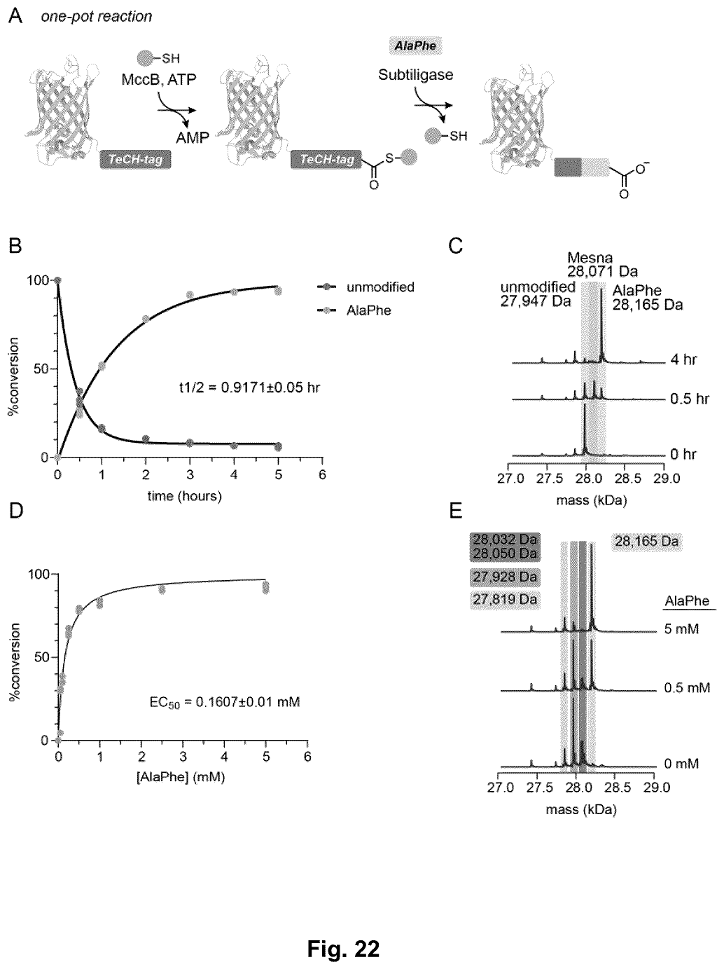

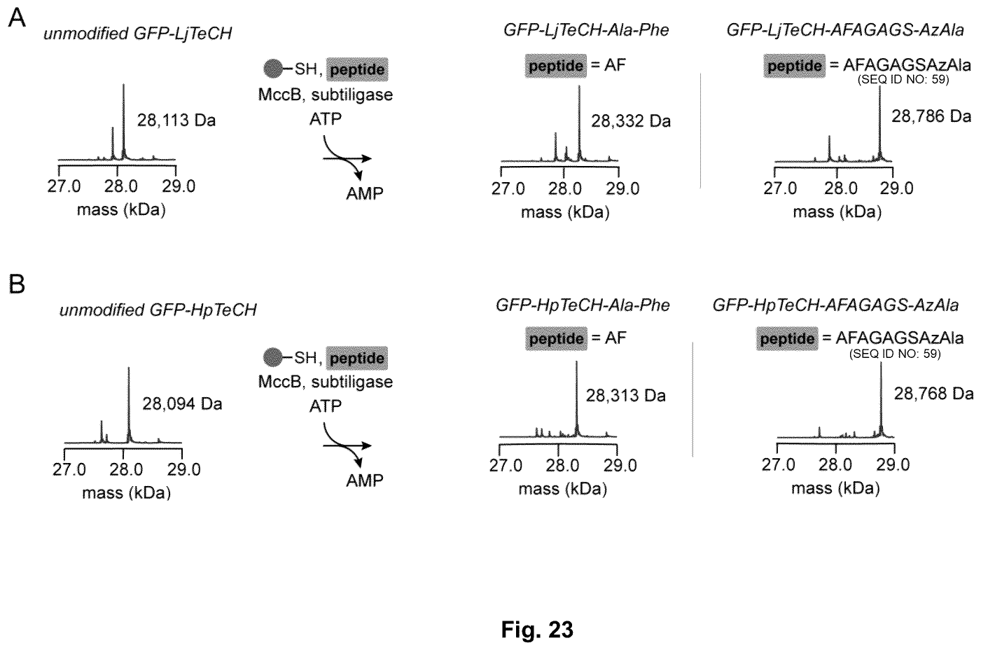

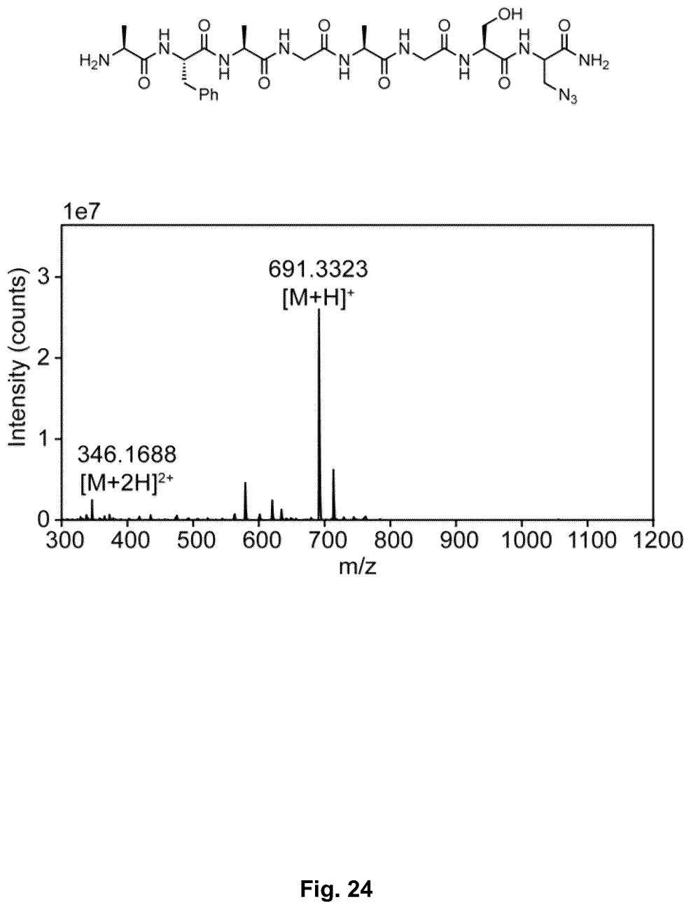

To test this hypothesis, we incubated TeCH-tagged GFP (50 mM) with M ccB (5 mM), ATP (5 mM), Mesna (5 mM), subtiligase (5 mM), and AFA GA GS-azidolysine (SEQ ID NO: 57) (5 mM, Table 4), which contains an azide for downstream modification using click chemistry. We found that GFP could be modified efficiently with this peptide (FIG. 20B, FIG. 21A). A timecourse indicated that t1/2 for the reaction was 0.9+0.1 h (FIG. 22). GFP variants bearing TeCH-tag sequences derived from H. pylori and L. johnsonii could also be efficiently modified by enzyme-catalyzed EPL (FIG. 3, FIG. 24, FIGS. 21B-21C). To test whether the efficiency of the ligation reaction depends on the protein substrate, we tested the reaction on our panel of TeCH-tag fusion proteins (MBP, PTP1B 1-321, a-GFP rAb, protein L, and zEGFR) (FIG. 20C, FIGS. 21D-21H). We found that all the proteins tested could be efficiently ligated to AFAGAGS-azidolysine (SEQ ID NO: 57) in high yields, highlighting the general utility of M ccB for driving subtiligase-catalyzed peptide ligation.

To test the utility of bioconjugates synthesized with M ccB/TeCH-tag enzyme-catalyzed EPL method in a biological context, we constructed a HEK 293T cell line that expresses cell surface GFP under the control of a tetracycline/doxycycline-inducible promoter. We synthesized a Cy3-modified a-GFP rAb by using M ccB and subtiligase to ligate AFAGAGS-azidolysine (SEQ ID NO: 57) onto the C-terminal TeCH-tag fused to the heavy chain, followed by SPAAC with DBCO-Cy3 (FIG. 211). We then stained doxycycline (Dox)-induced cells and uninduced cells with the a-GFP rAb-Cy3 conjugate. We observed robust Cy3 staining that colocalized with GFP in the Dox-induced cells, while neither GFP signal nor Cy3 signal was observed in uninduced cells (FIG. 20D). These results demonstrate the utility of M ccB- and subtiligase-catalyzed bioconjugation for incorporating probes into antibodies while maintaining their ability to bind their targets.

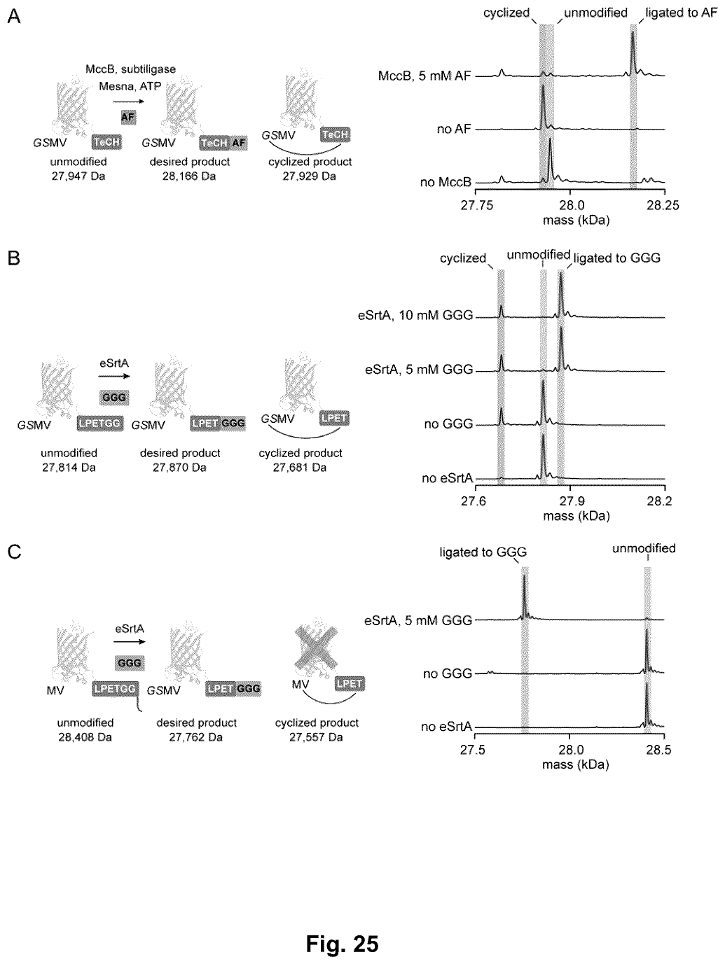

Similar to sortagging, the TeCH-tag/M ccB/subtiligase system achieves protein bioconjugation through formation of a peptide bond. We therefore sought to compare the efficiency of M ccB/subtiligase-catalyzed protein bioconjugation to sortase-catalyzed bioconjugation using the engineered sortase variant eSrtA. We initially replaced the TeCH-tag in our GFP construct with the eSrtA recognition sequence LPETGG (SEQ ID NO: 58). In contrast to the near-quantitative conversion to the desired ligation product catalyzed by M ccB, eSrtA (2.5 mM) catalyzed 75% ligation of GFP-LPETGG (50 mM) to a triglycine nucleophile (GGG, 5 mM), while 25% of GFP-LPETGG was cyclized based on the presence of an N-terminal Gly residue that could serve as an eSrtA substrate (FIG. 25, FIGS. 21J-21L). This intramolecular reaction could not be suppressed even when the GGG concentration was increased to 10 mM (FIG. 21M). We hypothesize that cyclization was effectively suppressed in the M ccB/subtiligase reaction in the presence of 5 mM ligation partner because the ligation product is no longer a substrate for M ccB. In contrast, the eSrtA-catalyzed reaction is reversible, and formation of the desired ligation product is governed by the position of the equilibrium between the intermolecular and intramolecular products. Although this intramolecular reaction could be blocked by using an alternative N-terminal sequence (FIG. 25, FIG. 21N-21P), the ability of M ccB/subtiligase-catalyzed bioconjugation to avoid this reaction highlights the utility of using irreversible steps to drive the reaction along an intended trajectory.

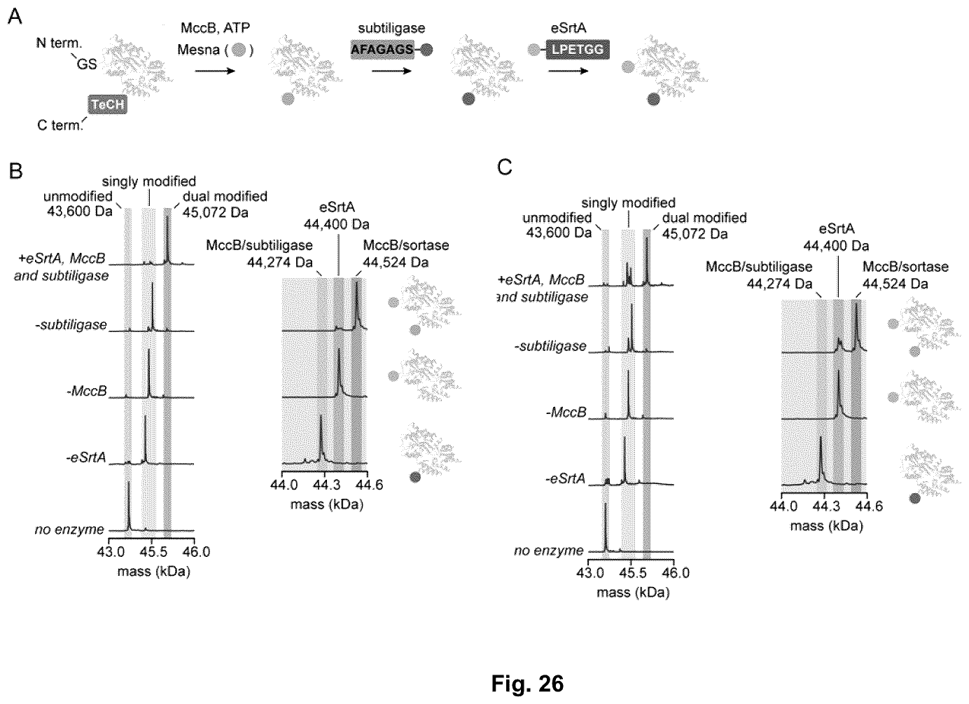

We next assessed whether M ccB/subtiligase and eSrtA can function in combination for dual functionalization of a single protein. As a test substrate, we used MBP modified at the N terminus with Gly-Ser and at the C terminus with a TeCH tag. We incubated this protein (25 mM) with eSrtA (2.5 mM), M ccB (5 mM), subtiligase (5 mM), AFAGAGS-azidoA la (SEQ ID NO: 59) (5 mM, M ccB/subtiligase substrate), and 5-FAM-LPETGG (SEQ ID NO: 60) (2 mM, eSrtA substrate) for 12 h at room temperature. In the presence of all three enzymes, we observed 72% conversion to dual modified protein, while only 5-FAM-LPET modification was observed in the absence of M ccB, only AFAGAGS-azidoAla (SEQ ID NO: 59) modification was observed in the absence of eSrtA, and 5-FAM-LPET/Mes modification was observed in the absence of subtiligase (FIG. 26, FIG. 21Q-21T). To optimize conversion to the dual N- and C-terminally modified product, we next tried a telescoping approach in which GS-MBP-TeCH (25 mM) was incubated with M ccB (5 mM), subtiligase (5 mM), and AFAGAGS-azidoA la (SEQ ID NO: 59) (5 mM, M ccB/subtiligase substrate) for 4 h at room temperature, followed by addition of eSrtA (2.5 mM) and 5-FAM-LPETGG (SEQ ID NO: 60) (2 mM) and incubation for an additional 4 h at room temperature (FIG. 26, FIG. 21U-21Y). Under telescoping conditions, we observed near-quantitative conversion of GS-MBP-TeCH to a species modified with both the FAM- and azide-bearing peptides. Notably, dual N- and C-terminal labeling using orthogonal sortase variants is currently limited by the incomplete orthogonality of these engineered variants in terms of the nucleophilic ligation partners that they accept. The application of M ccB/subtiligase therefore complements and expands the existing toolkit for complex bioconjugation applications.

### Application of a Promiscuous M ccB for Ub-Derived Peptide Thioester Synthesis

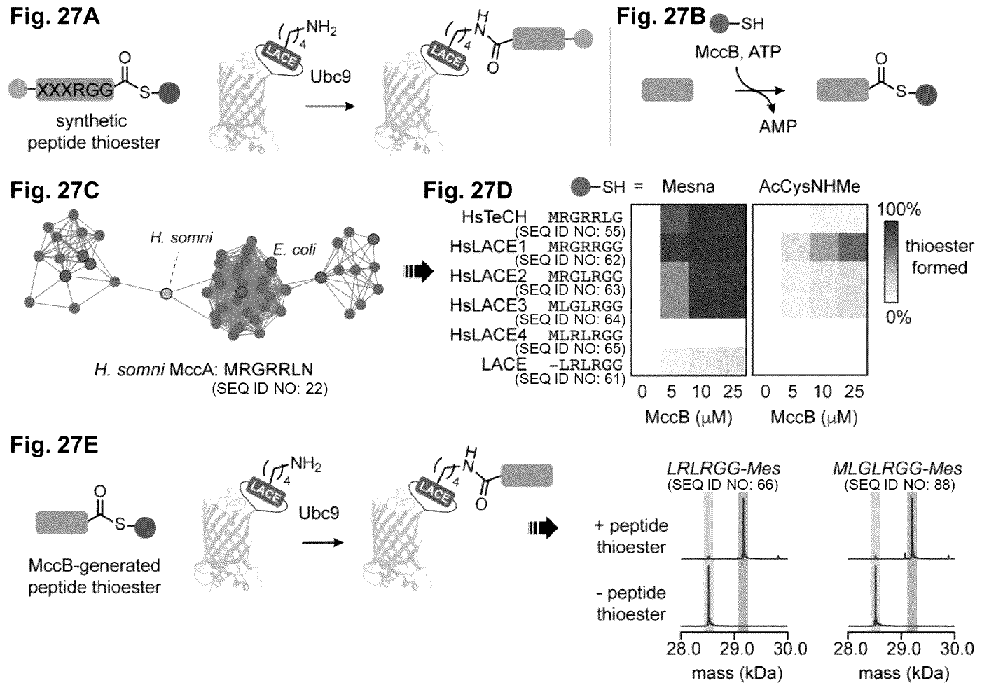

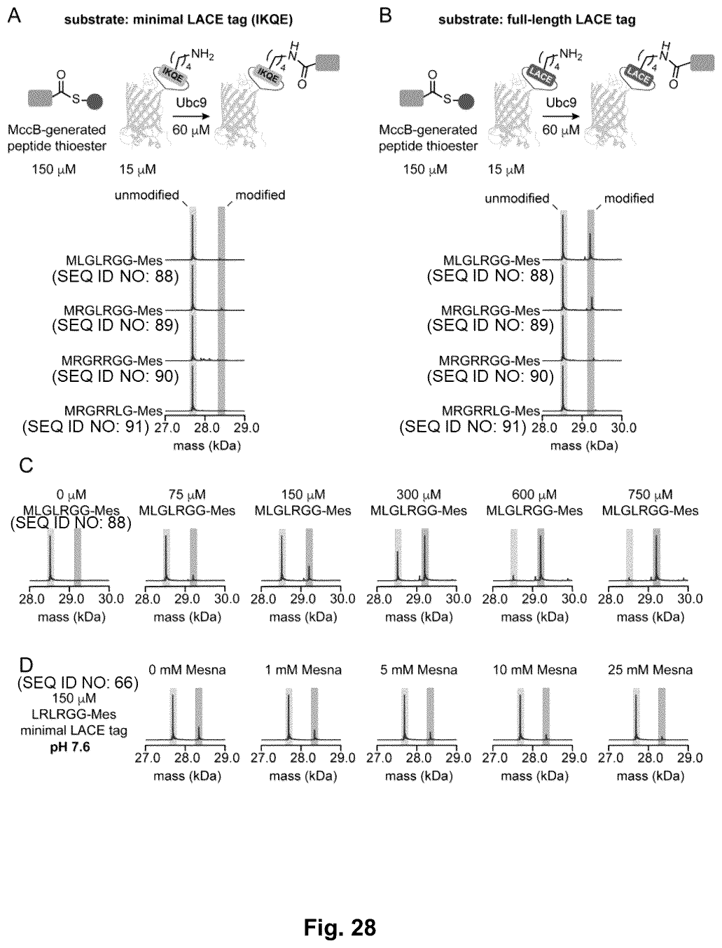

In our screen for mutually orthogonal M ccA/M ccB pairs, we observed that H. somni M ccB exhibited more cross-reactivity than homologs from E. coli, H. pylori, and L. johnsonii (FIG. 14B). We wondered whether HsMccB's expanded substrate tolerance might make it suitable for synthesis of thioesters derived from peptide sequences divergent from the native HsM ccA sequence. We chose to examine whether HsM ccB could be deployed to synthesize U b-derived peptide thioesters for lysine acylation using conjugating enzymes (LACE), a recently developed bioconjugation strategy. In LACE, a lysine within a genetically encoded tag of 4-13 residues (the LA CE tag) is recognized by the E2 SUMO-conjugating enzyme Ubc9 and modified with a peptide thioester derived from Ub (FIG. 27A). The typical peptide thioester sequence motif recognized by U bc9 is comprised of the six C-terminal residues of Ub, LRLRGG (SEQ ID NO: 61), with the final three amino acids representing a minimal motif for Ub loading.

We hypothesized that HsM ccB might be useful for thioesterification of these Ub-derived peptide based on its promiscuity as well as the basic nature of both the HsM ccA-N 7G and LRLRGG (SEQ ID NO: 61) peptides (FIG. 2. 27B and 27C). We synthesized a panel of peptides that introduce one amino acid variation at a time to convert HsM ccA-N 7G to a Ubc9 substrate and tested whether they could be thioesterified by HsM ccB. We found that HsM ccB catalyzed efficient (>99%) thioesterification of HsM ccA-N 7G (MRGRRLG; SEQ ID NO: 55), HsLACE1 (MRGRRGG; SEQ ID NO: 62), HsLACE2 (MRGLRGG; SEQ ID NO: 63), and HsLACE3 (M LGLRGG; SEQ ID NO: 64) with M esna as a thiol donor (FIG. 27D). HsLACE4 (MLRLRGG, SEQ ID NO: 65, <1% thioesterification) and LRLRGG (SEQ ID NO: 61) (18% thioesterification) were poor substrates. We also tested A cCysNHM e, the most widely used thiol donor for synthesis of Ubc9 peptide thioester substrates and found that it was a poor thiol donor for HsM ccB-catalyzed thioesterification (FIG. 27D).

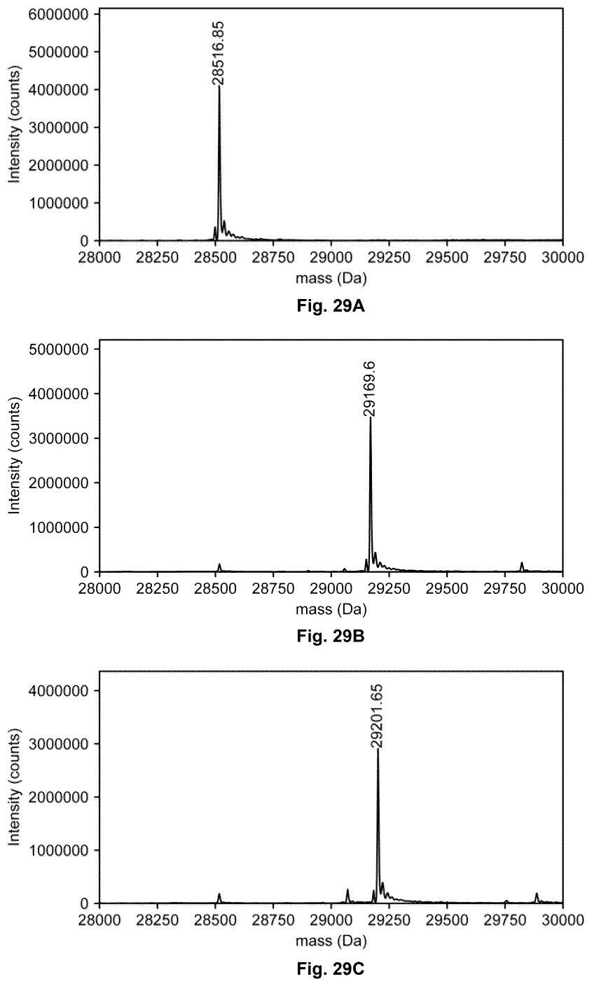

We next tested the ability of our panel of HsM ccB-generated Mes thioesters to serve as substrates for Ubc9-catalyzed lysine acylation of a LACE tag introduced into an internal site of GFP (following D173). In our initial screen, we found that efficient modification of the LACE tag only occurred when the peptide thioester contained at least the four C-terminal residues of Ub (LRGG) (FIG. 28). We therefore proceeded with MLGLRGG (SEQ ID NO: 64), initially attempting one-pot and telescoped HsM ccB/U bc9 GFP-LACE modification reactions, but we observed low conversion (1-15%) to modified GFP-LACE (FIG. 28). We attribute these low yields to inhibition of the U bc9-catalyzed reaction by the excess free thiol required for HsM ccB thioester synthesis (FIG. 28). We next incorporated C18 spin column cleanup of the crude reaction to remove excess thiol into our workflow. After thiol removal, peptide thioesters were used directly in the Ubc9 GFP-LACE modification reaction and gave 94% modification of the LACE-tagged protein, similar to a synthetic peptide thioester control, LRLRGG-Mes (SEQ ID NO: 66) (FIG. 27E, FIGS. 29A-29C). HsM ccB therefore provides an alternative enzymatic route to peptide thioesters that are key reagents for chemical biology and that have previously been accessible only through traditional chemical synthesis. This enzymatic approach lowers the barrier for deployment of bioorganic chemistry approaches such as LACE tag for interdisciplinary scientists who may lack extensive expertise in synthetic chemistry.

### DISCUSSION

We designed the M ccB/TeCH-tag system to mimic the chemical logic of peptide bond synthesis in biological systems to drive protein and peptide bioconjugation reactions to high yield. M ccB/TeCH-tag can be used in the context of enzyme-catalyzed EPL for ATP-dependent thioester regeneration, driving the reaction equilibrium toward the desired ligation product and away from the dead-end thioester hydrolysis product formed by adventitious subtiligase reactivity. Our system avoids the reversibility of transpeptidases, such as sortase and asparaginyl endopeptidases, in which the desired ligation products are also transpeptidase substrates. Because this limitation is based on the position of the equilibrium, it cannot be overcome through transpeptidase engineering. Although depsipeptide (ester) and thiodepsipeptide (thioester) substrates have been used to drive transpeptidation, these substrates must be chemically synthesized, cannot be regenerated, and are mainly useful for N-terminal rather than C-terminal labeling.

The MccB/TeCH-tag system couples ATP cleavage to C-terminal activation via formation of a peptidyl-O-AMP for high-yield in vitro protein modification. Although acyl-O-AM Ps are reactive electrophiles analogous to the acid chlorides and acid anhydrides often used in organic synthesis, they have not typically been viewed as modular intermediates that can be deployed for synthesis of modified proteins and peptides. We took advantage of our understanding of the enzymatic reaction mechanism of M ccB to design a system for peptidyl-O-AMP synthesis that can be integrated with protein chemistry toolbox in modular fashion for bioconjugate synthesis. Introduction of the N 7G substitution to MccA abolishes its ability to serve as a precursor to the antimicrobial compound microcin C7, but still supports the formation of a C-terminally O-AM Pylated electrophile. We showed that MccA-N7G-O-AMP can react with alkoxyamines, hydrazines, amines, and thiols to form oximes, hydrazides, amides, and thioesters, respectively. We anticipate that this reactivity can easily be extended to other nucleophiles for synthesis of C-terminally modified proteins. For example, ammonia could be used as a nucleophile for modification of proteins by C-terminal amidation, which has recently been shown to target proteins for ubiquitin modification by SCF/FBX 031 and proteasomal degradation (M uhar et al. “C-terminal amides mark proteins for degradation via SCF-FBX 031.” Nature 1-9, 2025). Other classes of nucleophiles such as alcohols could also capture the M ccA-N 7G-O-AM P intermediate for installation of protein C-terminal esters, which are present in prenylated proteins including Ras GT Pases.

We show that capture of M ccA-N 7G-O-AM P with thiol nucleophiles is particularly useful in protein bioconjugation as it converts the hydrolytically unstable peptidyl-O-AM P to a kinetically stable yet thermodynamically activated thioester. In biology, C-terminal thioesters function in enzymatic catalysis, serve as intermediates in protein splicing, and enable the installation of post-translational modifications including ubiquitin and ubiquitin-like proteins. Although nature has evolved several strategies to generate C-terminal thioesters, only one, intein-mediated protein splicing, had previously been harnessed as a tool for producing recombinant protein C-terminal thioesters that serve as versatile intermediates for synthesis of chemically tailored proteins (Thompson et al. “Chemoenzymatic semisynthesis of proteins.” Chem. Rev. 120, 3051-3126, 2020). Recombinant C-terminal thioesters can be deployed for native chemical ligation to N-terminal Cys peptides in expressed protein ligation (EPL) or can be used as substrates for the engineered peptide ligase subtiligase in enzyme-catalyzed EPL. These strategies have enabled precise manipulation of protein structure to advance our understanding of a broad range of biological questions. The M ccB/TeCH-tag system expands the toolbox for direct C-terminal thioester synthesis from unactivated protein a-carboxylates and therefore represents a broadly applicable tool for protein bioconjugation.

Our results indicate that M ccB s from E. coli, H. pylori, and L. johnsonii are epitope-specific, while M ccB from H. somni is more promiscuous. Epitope-specific bioconjugation enzymes are valuable tools for protein modification, enabling installation of probes, payloads, and modifications that cannot be genetically encoded. In previous work, bacterial sortases have been widely applied for epitope-specific bioconjugation based on their selectivity for an LPXTG motif (SEQ ID NO: 67) (Fottner et al. “Site-specific ubiquitylation and SUM Oylation using genetic-code expansion and sortase.” Nat. Chem. Biol. 15, 276-284, 2019). However, synthesis of complex bioconjugates can be limited by the relatively small number of available epitope-enzyme pairs, and few orthogonal sortase/sorting motif pairs have been identified in nature (Antos et al. “Site-specific N- and C-terminal labeling of a single polypeptide using sortases of different specificity.” J. Am. Chem. Soc. 131, 10800-10801, 2009). As a result, development of new orthogonal sortases has required intensive protein engineering efforts. In contrast, the natural diversity of M ccA/M ccB pairs enabled us to readily generate two orthogonal tools for epitope-specific protein bioconjugation. We also developed the more promiscuous H. somni for enzymatic synthesis of ubiquitin-derived peptide thioesters for the LACE system from unactivated peptides. Although we characterized four M ccB/TeCH-tags, there are at least 31 distinct annotated heptapeptide M ccA sequences with potential utility for protein bioconjugation (Zukher et al. “Reiterative synthesis by the ribosome and recognition of the N-terminal formyl group by biosynthetic machinery contribute to evolutionary conservation of the length of antibiotic microcin C peptide precursor.” mBio 10, e00768-19, 2019; B antysh et al. “Enzymatic synthesis of bioinformatically predicted microcin C-like compounds encoded by diverse bacteria.” mBio 5, e01059-14, 2014). Future genome mining and experimental approaches to identify orphan M ccB substrates have the potential to further expand the number of available M ccB/TeCH-tag systems to enable the design of tailor-made bioconjugation enzymes that recognize user-defined sequences as well as more promiscuous enzymes for general C-terminal thioesterification.

Advances in our ability to construct modified proteins have expanded the frontiers of our understanding of how post-translational modifications regulate transcription, transduce cellular signals, and go awry in neurodegenerative disease; and have propelled our ability to probe biochemical and biophysical function through the installation of chemical probes and payloads that cannot be genetically encoded. The M ccB/TeCH-tag system provides a new method for C-terminal protein and peptide activation that vastly expands the toolkit for protein bioconjugation. M ccB/TeCH-tag is broadly useful based on its ability to drive peptide bond-forming reactions to high yield by coupling them to ATP cleavage and can be integrated with existing protein chemistry technologies to fuel biological discovery.

### Methods

Key chemicals and materials. Reagents screened for nucleophilic capture of M ccB-activated C termini are listed in Table 3. M aleimide, Dibenzocyclooctyne-PEG4-biotin, and A laPhe dipeptide were purchased from Sigma Aldrich. TCEP hydrochloride was purchased from Gold Biotechnology. Cyanine5 maleimide was purchased from Lumiprobe. EZ-Link-maleimide-PEG2-biotin was purchased from Thermo Fisher Scientific. Protected amino acids, 1-hydroxybenzotriazole, 4-alkoxybenzyl alcohol resins, and Rink amide resin for solid-phase peptide synthesis were purchased from Chem Impex International.

Solid phase peptide synthesis. M ccA C-terminal variant peptides with C-terminal carboxylate groups were synthesized using fluorenylmethyloxycarbonyl (Fmoc) chemistry on 4-alkoxy-benzyl-alcohol resin preloaded with the required C-terminal amino acid (Chem Impex International). All other peptides with C-terminal amides were synthesized using Rink A mide resin (Chem Impex International). Fmoc amino acids with reactive side chains were protected with acid-labile protecting groups as follows: Asp (OtBu); Glu (OtBu); His (Trt); Lys (Boc); Asn (Trt); Gln (Trt); Arg (Pbf); Ser (tBu); Thr (tBu); Trp (Boc); Tyr (tBu). Fmoc groups were deprotected via 30-minute incubation in 20% methylpiperidine in DMF (20% v/v). Coupling steps were performed with 5 molar equivalents of the appropriate Fmoc-protected amino acid, 5 molar equivalents of diisopropylcarboiimide (DIC), and 5 molar equivalents of 1-hydroxy-benzotriazole (HOBt). Completed peptides were cleaved from the resin via incubation in a cocktail containing 95% trifluoroacetic acid, 2.5% triisopropylsilane, and 2.5% water. Peptides were concentrated under a stream of nitrogen and precipitated with 10 volumes of diethyl ether. Precipitated peptides were washed with additional diethyl ether and allowed to dry. The resulting crude product was purified using an Agilent 1260 Infinity II HPLC fitted with a semi-preparative ZORBAX Eclipse XDB-C18, 9.4× 250 mm, 5 μm column. Crude products were separated using a 30-minute gradient from 0 to 100% B (A=0.1% trifluoroacetic acid in water, B=acetonitrile). Selected fractions were lyophilized, resuspended in water, and stored at −20° C. The positional scanning peptide library for M ccB specificity characterization in FIGS. 14A-14F was purchased from Peptide2.0. ESI-MS data for synthetic peptides are shown in FIGS. 13 and 24 or raw data have been deposited to Dryad under DOI: 10.5061/dryad.c59zw3rkb (MccA positional scanning peptide library).

Mass spectrometry. Electrospray ionization liquid chromatography mass spectrometry (ESI-LC-MS) analysis was performed on an Agilent 6230B time of flight (TOF) mass spectrometer. Samples containing peptide substrates were separated on an Agilent ZORBAX Eclipse XDB-C18, Solvent Saver Plus, 3×150 mm, 3.5 μm column using a 5-minute gradient from 0 to 100% B (A=0.1% formic acid in water, B=acetonitrile). Extracted ion chromatograms were generated using Agilent MassHunter Qualitative Analysis v10.0 and Agilent TOF Quantitative Analysis v11.0. Samples containing intact protein substrates were separated on a PLRP-S 1000 Å, 50×1 mm, 5 μm column at 80° C. using a 3.9-minute gradient from 20 to 60% B (A=0.1% formic acid in water, B=acetonitrile). The maximum entropy charge deconvolution algorithm in Agilent M assHunter BioConfirm v10.0 was used to determine the neutral mass of intact proteins. For mixtures of TeCH-tagged proteins, the pM od algorithm was applied for charge deconvolution in Agilent M assHunter BioConfirm v10.0.

Molecular biology and plasmid construction. Plasmids were constructed using standard Gibson assembly cloning methods with E. coli XL10 as the cloning host. Oligonucleotides were purchased from Integrated DNA Technologies, and plasmid sequences were confirmed via Sanger Sequencing performed by Quintara Biosciences or Functional Biosciences. Plasmid maps have been deposited in Dryad under DOI: 10.5061/dryad.c59zw3rkb.

pBH4-His-TEV-M ccB. E. coli codon-optimized genes encoding M ccBs from E. coli, H. pylori, L. johnsonii, and H. somni were purchased from Integrated DNA Technologies. The genes were inserted into the pBH4 vector between the BamHI and Notl restriction sites using Gibson assembly to generate constructs with an N-terminal His tag followed by a TEV protease cleavage site.

pBH4-His-TEV-cysteine-free (cf)-GFP-MccA fusions. Cysteine-free eGFP was amplified from ss-cfSGFP2 (Addgene #37535). Primers were used to add a C-terminal linker and M ccA or M ccA-N 7G sequence. PCR products were inserted into pBH 4 between BamHI and Notl restriction sites using Gibson assembly to generate a construct with an N-terminal His tag and a TEV protease cleavage site.

pBH4-His-TEV-MBP-EcTeCH. MBP was amplified from pRK 793 (Addgene #8827, a gift from Dr. David Waugh) using primers that added a C-terminal linker and TeCH tag (GGGSMRTGNAG; SEQ ID NO: 68). The PCR product was inserted between BamHI and Notl restriction sites using Gibson assembly to generate a construct with an N-terminal His tag and a TEV protease cleavage site.

pBH4-His6-TEV-PTP 1B 1-321. An E. coli codon-optimized gene encoding the PTP1B catalytic domain (residues 1-321) followed by a TeCH tag was purchased from Twist Bioscience. The gene was inserted between the BamHI and Notl sites of pBH4.

pET28a-His6-protein L-EcTeCH. An E. coli codon-optimized gene encoding His6-protein L with a C-terminal TeCH tag was purchased from Twist Bioscience. The gene was inserted between the N col and N del sites of pET 28a.

pP SL 937-anti-GF P-rAB—HC-TeCH. pPSL 937-anti-GFP rAb was a gift from James A. Wells. A gene encoding the final 19 amino acids of the heavy chain of the rA b followed by a TeCH tag was purchased from Twist Bioscience. The gene was inserted between the two Sall sites of pPSL 937.

pBH4-His-TEV-ZEGFR-EcTeCH. A gene encoding zEGFR followed by a C-terminal TeCH tag was purchased from Integrated DNA Technologies. The gene was inserted between the BamHI and Notl sites to generate a construct with an N-terminal His tag followed by a TEV protease cleavage site.

pBS42-pre-pro-stabiligase-His6. Construction of pBS42-pre-pro-stabiligase-His6 was described previously. The plasmid was a gift from James A. Wells.

pET29-eSrtA. pET 29a-eSrtA (Addgene #75144) was a gift from David Liu.

pcDNA5/F RT/TO-Igk-eGFP-PDGFRTM. A synthetic gene encoding eGFP with an N-terminal Igk signal peptide and a C-terminal PDGF receptor β-chain transmembrane domain to target eGFP to the cell surface was ordered from Twist Bioscience. The gene was inserted between the Ncol and Notl sites of pcDNA5/FRT/TO (Thermo Fisher) using Gibson assembly.

pTXB1-cfGFP-TeCH-Mxe. pTX B1 was obtained as part of the IMPACT kit from New England Biolabs (catalog no. E6901S). cfGFP-TeCH was amplified from pBH4-cfGFP-EcM ccA-N 7G and inserted into the Ndel and Sapl sites of pTX B1.

pBH4-His-TEV-Ubc9. An E. coli codon-optimized gene encoding Ubc9 was purchased from Integrated DNA Technologies. The gene was inserted between the BamHI and Notl sites of pBH4 using Gibson assembly to generate a construct with an N-terminal His tag followed by a TEV protease cleavage site.

pBH4-His-TEV-GFP-D 173-LACE tag. Genes encoding previously reported GFP constructs with a minimal or full-length LACE tag sequence following D173 were purchased from Integrated DNA Technologies. The genes were inserted between the N col and Notl sites of pBH4.

Protein expression and purification. Protein expression and purification methods for each protein used in this study are described below. Following purification, the purity of proteins was analyzed by SDS-PAGE and ESI-MS (FIGS. 10A, 10C, 10E, 10G, 10I, 10K, 18A, 18C, 21J, 21N, 29A, 30A-30G).

### MccB

E. coli MccB
(SEQ ID NO: 69)
MGHHHHHHDYDIPTTENLYFQGSMDYILGRYVKIARYGSGGLVGG
GGKEQYVEDLALWENIIKTAYCFITPSSYTAALETVNIPEKDFSN
CFRFLKENFFIIPSEYNNSTENNRYSRNFLHYQSYGANPVLVQDK
LKDAKVVILGCGGIGNHVSVILATSGIGEIILIDNDQIENTNLTR
QVLFSENDVGKNKTEVIKRELLKRNSEISVSEIALNINDYTDLHK
VPEADIWVVSADHPFNLINWVNKYCVRANQPYINAGYVNDIAVFG
PLYVPGKTGCYECQKVVADLYGSEKENIDHKIKLINSRFKPATFA
PVNNVAAALCAADVIKFIGKYSEPLSLNKRIGIWSDEIKIHSQNM
GRSPVCSVCGNRM

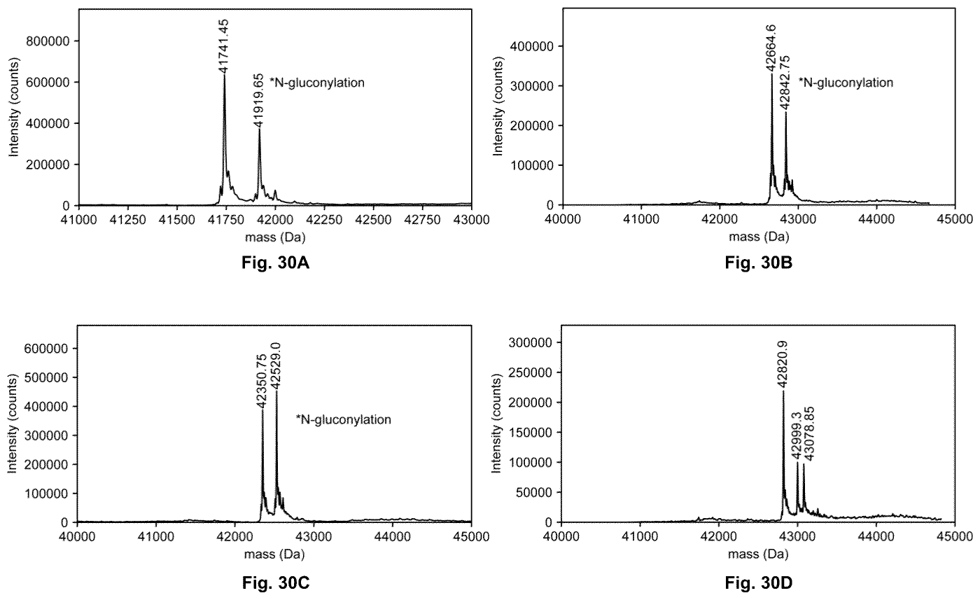

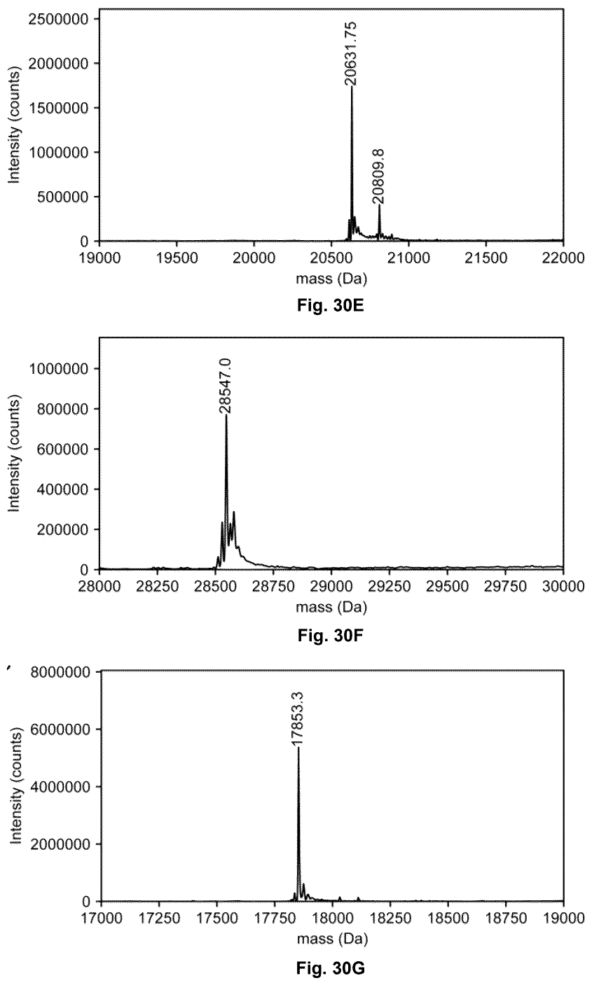

The italicized sequence was cleaved using TEV protease. ESI-MS data for the purified protein are shown in FIG. 30A.

Chemically competent E. coli BL21 (DE3) cells were transformed with pBH4-His-TEV-E. coli MccB for overexpression. LB starter cultures (15 mL, 50 μg/mL carbenicillin) were grown overnight and used to inoculate 1 L LB cultures (50 μg/mL carbenicillin). Cultures were incubated at 37° C. with vigorous shaking until OD 600 reached ˜0.6. Cultures were chilled on ice for 15 minutes prior to adding 0.1 mM IPTG (isopropyl-B-D-thiogalactopyranoside). Cultures were shaken an additional 24 hours at 16° C. Cell pellets were harvested by centrifugation at 4° C., resuspended in 40 mL lysis buffer (25 mM Tris, 500 mM NaCl, 10 mM M gCI2, pH 8.0). Cells were lysed by three passes through an Emulsiflex microfluidizer at 15000 psi, and the resulting lysate was centrifuged at 8000× g for 15 minutes at 4° C. Ni-NTA resin (1 mL) was added to the clarified lysate and His-tagged proteins were allowed to bind for 1 hour at 4° C. with gentle rocking. The Ni-NTA resin was collected by centrifugation at 500×g for 5 min, transferred to a 15 mL column, and washed with 15 mL wash buffer (20 mM Tris pH 8.0, 500 mM NaCl, 25 mM imidazole). Protein was eluted with 5 mL elution buffer (20 mM Tris pH 8.0, 500 mM NaCl, 200 mM imidazole) and dialyzed against wash buffer overnight at 4° C. The dialyzed protein was then concentrated using A micon centrifugal filter units (10,000 MWCO) and further purified by size-exclusion chromatography (Superdex Hiload 75, GE Healthcare) using storage buffer (25 mM Tris pH 8.0, 50 mM NaCl, 1 mM DTT, 10% glycerol). For M ccB/subtiligase experiments, glycerol was omitted from size-exclusion chromatography and storage buffers. Collected fractions were analyzed by Coomassie-stained SDS-PAGE. Fractions containing M ccB were pooled, concentrated, and flash frozen in liquid nitrogen. Protein concentrations were determined using absorbance at 280 nm and the protein extinction coefficient as calculated using ProtParam.

H. pylori M ccB
(SEQ ID NO: 70)
MGHHHHHHDYDIPTTENLYFQGSMQWYQTSFSACVGQTDTENIIG
LGTYQYCVDHNEFEKSLKLLVFLRMKKRMAEIKSFMETSKIEHNI
FDKLVANKLITSFILNPNDEQNFKNHLFIDLMSSKPELTIDNFKR
TIFIIIGCGGIGNFVSYALASFYPKKLILLDKDTVDFSNLNRQFL
FDKNYISQYKTSAIKQALSSRFSINIETVDDFASEDNLEEIFSKH
KKENLFGIVSGDNPNTVQLATRFFCKCRIPFLNIGYLNDISLIGP
FYIPSLSCCPFCHNSFALDDKKDGDENLDICLI

The italicized sequence was cleaved using TEV protease. ESI-MS data for the purified protein are shown in FIG. 30B.

L. johnsonii MccB
(SEQ ID NO: 71)
MGHHHHHHDYDIPTTENLYFQGSMFYKTSYLATGGCSNHQGILGV
GTKQYFVSEADYLKSLKILDFLLNKKTYDEVIKFCEKNNINKSIF
DTLVEHNLIVKENLYVEKKDDLNFKNKLYFHALGLNGNALAKEFA
DTTFVIVGOGGIGNFISFAIGSLSPRKIELIDGDKIEKSNLNRQF
LFTENDIGKYKVDVLKKNLVERNNKLSISEYKEYVSKEVLHNIFE
QNKKNKTLVILSGDSFSALSLTAKACVKSEIPFLNIGYLNDISAI
GPFYIPGISSCPFCHNALSISDDISSGHNESKILEDRINANNEAP
SSFTNNALAASMGIADIIEFLSHNYERINSLNKRFGINSATFEKY
VLEVNRDRKCEICSHGE

The italicized sequence was cleaved using TEV protease. ESI-MS data for the purified protein are shown in FIG. 30C.

H. somni MccB
(SEQ ID NO: 72)
MGHHHHHHDYDIPTTENLYFQGSMKYITSKHVFFDYLNENEFVIG
IGSNQEITNNKDYFNNCLNLCYFCINPKSISEILSFIKDNNIDIL
YFDKMKKMKFITKEIIDFNDRYSRNHLYYNALGYKIYDIQNKISK
SHILIVGAGGIGNICSYLLGTIGIKKLSIIDDDIVEESNLNRQFL
FREKDINKNKVETIKRELLSIRKDIIIDIFPEKLNKSILDKISQI
DLVICSADDEYCIDMINEFCCFNKIPLINVGYLNDISVIGPFYIP
KLEYSCCLCCDKSIYLENDVIDEKVKKIKSVTKAPSTIINNFFAG
AMLGSELIKFFARDYKSMQSINSVIGIHNKNFKYEEIKLAKNYNC
KYCGVNNETL

The italicized sequence was cleaved using TEV protease. ESI-MS data for the purified protein are shown in FIG. 30D.

Chemically competent E. coli BL21 (DE3) cells harboring the pGro7 chaperone plasmid (Takara Bio) were transformed with pBH4-His-TEV-H. pylori M ccB or pBH4-His-TEV-L. johnsonii MccB for overexpression. LB starter cultures (15 mL, 50 μg/mL carbenicillin and 25 μg/mL chloramphenicol) were grown overnight and used to inoculate 1 L LB cultures (50 μg/mL carbenicillin and 25 μg/mL chloramphenicol). Cultures were incubated at 37° C. with shaking at 200 rpm until OD 600 reached 0.5-0.7. Cultures were chilled on ice for 15 minutes prior to adding 0.25 mM IPTG (isopropyl-β-D-thiogalactopyranoside) and 2 mg/L arabinose. Cultures were shaken an additional 20 hours at 18° C. Cell pellets were harvested by centrifugation at 4° C., resuspended in 40 mL lysis buffer (25 mM Tris, 500 mM NaCl, 10 mM M gCl2, pH 8.0). Cells were lysed by three passes through an Emulsiflex microfluidizer at 15,000 psi, and the resulting lysate was centrifuged at 8,000×g for 15 minutes at 4° C. Ni-NTA resin (1 mL) was added to the clarified lysate and His-tagged proteins were allowed to bind for 1 hour at 4° C. with gentle rocking. The Ni-NTA resin was collected by centrifugation at 500×g for 5 min, transferred to a 15 mL column, and washed with 15 mL wash buffer (20 mM Tris pH 8.0, 500 mM NaCl, 25 mM imidazole). Protein was eluted with 5 mL elution buffer (20 mM Tris pH 8.0, 500 mM NaCl, 200 mM imidazole) and dialyzed against 20 mM Tris pH 8.0, 500 mM NaCl overnight at 4° C. The dialyzed protein was then concentrated using A micon centrifugal filter units (10,000 MWCO). Collected protein was analyzed by Coomassie-stained SDS-PAGE. Single-use aliquots were flash frozen in liquid nitrogen. Protein concentrations were determined using absorbance at 280 nm and the protein extinction coefficient as calculated using ProtParam. ESI-M S data for the purified proteins are shown in FIGS. 30B-30D.

### Bioconjugation Enzymes and Protein Substrates

Bioconjugation tags/epitopes are underlined.

cfGFP-EcTeCH
(SEQ ID NO: 73)
MGHHHHHHDYDIPTTENLYFQGSMVSKGEELFTGVVPILVELDGD
VNGHKFSVSGEGEGDATYGKLTLKFISTTGKLPVPWPTLVTTLTY
GVQMFARYPDHMKQHDFFKSAMPEGYVQERTIFFKDDGNYKTRAE
VKFEGDTLVNRIELKGIDFKEDGNILGHKLEYNYNSHNVYITADK
QKNGIKANFKIRHNIEDGGVQLADHYQQNTPIGDGPVLLPDNHYL
STQSKLSKDPNEKRDHMVLLEFVTAAGITLGMDELYKGGGGMRTG
NAG

The italicized sequence was cleaved using TEV protease. ESI-MS data for the purified protein are shown in FIG. 10A.

MBP-EcTeCH
(SEQ ID NO: 74)
MGHHHHHHDYDIPTTENLYFQGSMKIEEGKLVIWINGDKGYNGLA
EVGKKFEKDTGIKVTVEHPDKLEEKFPQVAATGDGPDIIFWAHDR
FGGYAQSGLLAEITPDKAFQDKLYPFTWDAVRYNGKLIAYPIAVE
ALSLIYNKDLLPNPPKTWEEIPALDKELKAKGKSALMFNLQEPYF
TWPLIAADGGYAFKYENGKYDIKDVGVDNAGAKAGLTFLVDLIKN
KHMNADTDYSIAEAAFNKGETAMTINGPWAWSNIDTSKVNYGVTV
LPTFKGQPSKPFVGVLSAGINAASPNKELAKEFLENYLLTDEGLE
AVNKDKPLGAVALKSYEEELAKDPRIAATMENAQKGEIMPNIPQM
SAFWYAVRTAVINAASGRQTVDEALKDAQTNSSSNNNNNNNNNNL
GIEGRGGGGGMRTGNAG

The italicized sequence was cleaved using TEV protease. ESI-MS data for the purified protein are shown in FIG. 10E.

Protein L-EcTeCH
(SEQ ID NO: 75)
MHHHHHHKEETPETPETDSEEEVTIKANLIFANGSTQTAEFKGTF
EKATSEAYAYADTLKKDNGEYTVDVADKGYTLNIKFAGKEKTPEE
PKEEVTIKANLIYADGKTQTAEFKGTFEEATAEAYRYADALKKDN
GEYTVDVADKGYTLNIKFAGKEKTPEEPKEEVTIKANLIYADGKT
QTAEFKGTFEEATAEAYRYADLLAKENGKYTVDVADKGYTLNIKF
AGKEKTPEEPKEEVTIKANLIYADGKTQTAEFKGTFAEATAEAYR
YADLLAKENGKYTADLEDGGYTINIRFAGKKVDEKPEEKEQVTIK
ENIYFEDGTVQTATFKGTFAEATAEAYRYADLLSKEHGKYTADLE
DGGYTINIRFAGGGGSGGGSMRTGNAG

ESI-M S data for the purified protein are shown in FIG. 10K.

PTP 1B (1-321)-EcTeCH
(SEQ ID NO: 76)
MGHHHHHHDYDIPTTENLYFQGSMEMEKEFEQIDKSGSWAAIYQD
IRHEASDFPCRVAKLPKNKNRNRYRDVSPFDHSRIKLHQEDNDYI
NASLIKMEEAQRSYILTQGPLPNTCGHFWEMVWEQKSRGVVMLNR
VMEKGSLKCAQYWPQKEEKEMIFEDTNLKLTLISEDIKSYYTVRQ
LELENLTTQETREILHFHYTTWPDFGVPESPASFLNFLFKVRESG
SLSPEHGPVVVHCSAGIGRSGTFCLADTCLLLMDKRKDPSSVDIK
KVLLEMRKFRMGLIQTADQLRFSYLAVIEGAKFIMGDSSVQDQWK
ELSHEDLEPPPEHIPPPPRPPKRILEPHNGGGGMRTGNAG

The italicized sequence was cleaved using TEV protease. ESI-MS data for the purified protein are shown in FIG. S10G.

His-Tev-GFP-LPETGG
(SEQ ID NO: 77)
MGHHHHHHDYDIPTTENLYFQGSMVSKGEELFTGVVPILVELDGD
VNGHKFSVSGEGEGDATYGKLTLKFISTTGKLPVPWPTLVTTLTY
GVOMFARYPDHMKQHDFFKSAMPEGYVQERTIFFKDDGNYKTRAE
VKFEGDTLVNRIELKGIDFKEDGNILGHKLEYNYNSHNVYITADK
QKNGIKANFKIRHNIEDGGVQLADHYQQNTPIGDGPVLLPDNHYL
STQSKLSKDPNEKRDHMVLLEFVTAAGITLGMDELYKGGGGLPET
GG

The italicized sequence was cleaved using TEV protease. ESI-MS data for the purified protein are shown in FIG. 21J.

His-SUMO-GFP-LPETGG
(SEQ ID NO: 78)
MGHHHHHHDYDIPTTENLYFQGSSDSEVNQEAKPEVKPEVKPETH
INLKVSDGSSEIFFKIKKTTPLRRLMEAFAKRQGKEMDSLRFLYD
GIRIQADQTPEDLDMEDNDIIEAHREQIGGMVSKGEELFTGVVPI
LVELDGDVNGHKFSVSGEGEGDATYGKLTLKFICTTGKLPVPWPT
LVTTLTYGVQCFSRYPDHMKQHDFFKSAMPEGYVQERTIFFKDDG
NYKTRAEVKFEGDTLVNRIELKGIDFKEDGNILGHKLEYNYNSHN
VYIMADKQKNGIKVNFKIRHNIEDGSVQLADHYQQNTPIGDGPVL
LPDNHYLSTQSALSKDPNEKRDHMVLLEFVTAAGITLGMDELYKG
GGGLPETGGAAASRSGC

The italicized portion was cleaved with SUMO protease Ulp1 purchased from Trialtus Bioscience according to the manufacturer's instructions. ESI-MS data for the purified protein are shown in FIG. 21N.

The appropriate plasmid was transformed into chemically competent E. coli BL21 (DE3) cells for overexpression. LB starter cultures (15 mL, 50 μg/mL carbenicillin for pBH4 backbone or 50 μg/mL kanamycin for pET 28a backbone) were grown overnight and used to inoculate 1 L LB cultures (50 μg/mL carbenicillin). Cultures were incubated at 37° C. with vigorous shaking until OD 600 reached ˜0.6. Cultures were chilled on ice for 15 minutes prior to adding 0.4 mM IPTG (isopropyl-B-D-thiogalactopyranoside). Cultures were shaken an additional 16 hours at 18° C. Cell pellets were harvested by centrifugation at 4° C., resuspended in 40 mL wash buffer (50 mM sodium phosphate, 300 mM NaCl, 20 mM imidazole, pH 8.0). Cells were lysed by three passes through an Emulsiflex microfluidizer at 15000 psi, and the resulting lysate was centrifuged at 8000×g for 15 minutes at 4° C. Ni-NTA resin was added to the clarified lysate and allowed to bind for 1 hour at 4° C. with gentle rocking. The Ni-NTA resin was collected by centrifugation at 500× g for 5 min, transferred to a 15 mL column, and washed with 15 mL wash buffer. Protein was eluted with 5 mL elution buffer (50 mM sodium phosphate, 300 mM NaCl, 250 mM imidazole, pH 8.0) and dialyzed overnight at 4° C. against wash buffer containing 1 mM DTT. To remove the His tag, TEV protease was added to the dialysis tubing at a ratio of 1:50 protease: substrate. After 16-24 hours digestion, the protein solution was passed through 2 mL Ni-NTA resin equilibrated with wash buffer. The resulting protein was then concentrated using A micon centrifugal filter units (10,000 M W CO) and further purified by size-exclusion chromatography (Superdex Hiload 75, GE Healthcare) using storage buffer (20 mM Tris pH 8.0, 150 mM NaCl, 10% glycerol). Glycerol was omitted from the storage buffer for experiments with M ccB/subtiligase. Collected fractions were analyzed by Coomassie-stained SDS-PAGE. Fractions containing the correct protein molecular weight were pooled, concentrated, and flash frozen in liquid nitrogen. Protein concentrations were determined using absorbance at 280 nm and the protein extinction coefficient as calculated using ProtParam.

GFP-TeCH-Mes thioester from GFP-TeCH-MxeGyrA
(SEQ ID NO: 79)
MVSKGEELFTGVVPILVELDGDVNGHKFSVSGEGEGDATYGKLTL
KFISTTGKLPVPWPTLVTTLTYGVQMFARYPDHMKQHDFFKSAMP
EGYVQERTIFFKDDGNYKTRAEVKFEGDTLVNRIELKGIDFKEDG
NILGHKLEYNYNSHNVYITADKQKNGIKANFKIRHNIEDGGVQLA
DHYQQNTPIGDGPVLLPDNHYLSTQSKLSKDPNEKRDHMVLLEFV
TAAGITLGMDELYKGGGGMRTGNAGCITGDALVALPEGESVRIAD
IVPGARPNSDNAIDLKVLDRHGNPVLADRLFHSGEHPVYTVRTVE
GLRVTGTANHPLLCLVDVAGVPTLLWKLIDEIKPGDYAVIQRSAF
SVDCAGFARGKPEFAPTTYTVGVPGLVRFLEAHHRDPDAQAIADE
LTDGRFYYAKVASVTDAGVQPVYSLRVDTADHAFITNGFVSHATG
LTGLNSGLTTNPGVSAWQVNTAYTAGQLVTYNGKTYKCLQPHTSL
AGWEPSNVPALWQLQ

The italicized portion was removed by intein self-splicing. ESI-MS data for the purified protein are shown in FIG. 10C.

GFP-TeCH-Mxe GyrA intein fusion protein was purified according to IMPACT Kit instruction manual (NEB #E 6901S). Briefly, chemically competent E. coli BL21 (DE3) cells were transformed with pTXB1-Mxe-GFP-TeCH for overexpression. LB starter culture (15 mL, 50 μg/mL carbenicillin) were grown overnight and used to inoculate 1 L LB culture (50 μg/mL carbenicillin). Culture was incubated at 37° C. with vigorous shaking until OD600 reached ˜0.5. Culture was chilled on ice for 15 minutes prior to the addition of 0.4 mM IPTG (isopropyl-B-D-thiogalactopyranoside). Expression was carried out at 15° C. overnight to help increase the cleavage efficiency of the intein. Cell pellets were then harvested by centrifugation at 4° C., resuspended in 100 ml of ice-cold column buffer (20 mM HEPES, 500 mM NaCl, pH 8.5). Cells were lysed by three passes through an Emulsiflex microfluidizer at 15000 psi, and the resulting lysate was centrifuged at 8000× g for 15 minutes at 4° C. (unless otherwise noted the next steps were performed at 4° C.). The clarified lysate was then loaded onto a chitin column. Before loading the crude cell extract, the chitin resin bed was washed with 10 column volumes of the column buffer (20 mM HEPES, 500 mM NaCl, pH 8.5). Following loading, the column was washed with 20 bed volumes of the column buffer. To induce on-column cleavage, the column was quickly flushed with 3 column volumes of cleavage buffer containing Mesna as the thiol reagent (20 mM HEPES, 500 mM NaCl, 50 mM Mesna, pH 8.5). The flow was stopped after the quick flush and the column was incubated at 23° C. for 40 hours to ensure maximum cleavage efficiency. Protein was eluted with the column buffer by resuming the flow and dialyzed against storage buffer and concentrated to appropriate concentration using A micon centrifugal filter units (10,000 MWCO). Single-use aliquots were flash-frozen in liquid nitrogen and stored at −80° C.

U bc9
(SEQ ID NO: 80)
MGHHHHHHDYDIPTTENLYFQGSMSGIALSRLAQERKAWRKDHPF
GFVAVPTKNPDGTMNLMNWECAIPGKKGTPWEGGLFKLRMLFKDD
YPSSPPKCKFEPPLFHPNVYPSGTVCLSILEEDKDWRPAITIKQI
LLGIQELLNEPNIQDPAQAEAYTIYCQNRVEYEKRVRAQAKKFAP
S

ESI-M S data for the purified protein are shown in FIG. 30E.

GFP-LACE(D173)
(SEQ ID NO: 81)
MGHHHHHHDYDIPTTENLYFQGMRKGEELFTGVVPILVELDGDVN
GHKFSVRGEGEGDATNGKLTLKFICTTGKLPVPWPTLVTTLTYGV
QCFARYPDHMKQHDFFKSAMPEGYVQERTISFKDDGTYKTRAEVK
FEGDTLVNRIELKGIDFKEDGNILGHKLEYNFNSHNVYITADKQK
NGIKANFKIRHNVEDGSGPRKVIKMESEEGSGSVQLADHYQQNTP
IGDGPVLLPDNHYLSTQSVLSKDPNEKRDHMVLLEFVTAAGITHG
MDELYK

The italicized sequence was cleaved using TEV protease. ESI-MS data for the purified protein are shown in FIG. 29A.

The appropriate plasmid was transformed into chemically competent E. coli BL21 (DE3) cells for overexpression. LB starter cultures (15 mL, 50 μg/mL carbenicillin for pBH4 backbone or 50 μg/mL kanamycin for pET28a backbone) were grown overnight and used to inoculate 1 L LB cultures (50 μg/mL carbenicillin). Cultures were incubated at 37° C. with vigorous shaking until OD 600 reached ˜0.6. Cultures were chilled on ice for 15 minutes prior to adding 0.4 mM IPTG (isopropyl-B-D-thiogalactopyranoside). Cultures were shaken an additional 16 hours at 18° C. (GFP-LACE) or 4 h at 30° C. (Ubc9). Cell pellets were harvested by centrifugation at 4° C., resuspended in 40 mL wash buffer (50 mM HEPES, 350 mM NaCl, 20 mM imidazole, pH 8.0). Cells were lysed by three passes through an Emulsiflex microfluidizer at 15000 psi, and the resulting lysate was centrifuged at 8000×g for 15 minutes at 4° C. Ni-NTA resin was added to the clarified lysate and allowed to bind for 1 hour at 4° C. with gentle rocking. The Ni-NTA resin was collected by centrifugation at 500×g for 5 min, transferred to a 15 mL column, and washed with 15 mL wash buffer. Protein was eluted with 5 mL elution buffer (50 mM sodium phosphate, 300 mM NaCl, 250 mM imidazole, pH 8.0) and dialyzed overnight at 4° C. against wash buffer. To remove the His tag from GFP-LACE, TEV protease was added to the dialysis tubing at a ratio of 1:50 protease: substrate. A fter 16-24 hours digestion, the protein solution was passed through 2 mL Ni-NTA resin equilibrated with wash buffer. The resulting protein was then concentrated using Amicon centrifugal filter units (10,000 MWCO). Glycerol was omitted from the storage buffer for experiments with M ccB/subtiligase. Purified protein was analyzed by Coomassie-stained SDS-PAGE. Protein concentrations were determined using absorbance at 280 nm and the protein extinction coefficient as calculated using ProtParam. Single-use aliquots were flash frozen in liquid nitrogen and stored at −80° C.

Anti-GFP rAb Heavy Chain-TeCH
Light chain:
(SEQ ID NO: 82)
MKSLLPTAAAGLLLLAAQPAMASDIQMTQSPSSLSASVGDRVTIT
CRASQSVSSAVAWYQQKPGKAPKLLIYSASSLYSGVPSRFSGSRS
GTDFTLTISSLOPEDFATYYCQQSWGLITFGQGTKVEIKRTVAAP
SVFIFPPSDSQLKSGTASVVCLLNNFYPREAKVQWKVDNALQSGN
SQESVTEQDSKDSTYSLSSTLTLSKADYEKHKVYACEVTHQGLSS
PVTKSFNRGEC
Heavy chain:
(SEQ ID NO: 83)
MKKNIAFLLASMFVFSIATNAYAEISEVOLVESGGGLVQPGGSLR
LSCAASGFNISYYSIHWVRQAPGKGLEWVASIYPYYSSTSYADSV
KGRFTISADTSKNTAYLQMNSLRAEDTAVYYCARAGWVASSGMDY
WGQGTLVTVSSASTKGPSVFPLAPSSKSTSGGTAALGCLVKDYFP
EPVTVSWNSGALTSGVHTFPAVLQSSGLYSLSSVVTVPSSSLGTQ
TYICNVNHKPSNTKVDKKVEPKSCGGGGGMRTGNAG

ESI-MS data for the purified protein are shown in FIG. 10I.

pPSL 937-anti-GFP66 rAb-HC-TeCH was transformed into chemically competent C43 (DE3) Pro+pTUM protease-deficient E. coli cells for overexpression. LB starter cultures (5 mL, 50 μg/mL carbenicillin, 25 μg/mL chloramphenicol) were grown overnight and used to inoculate 1 L TB expression cultures (50 μg/mL carbenicillin, 25 μg/mL chloramphenicol, 0.05% w/v glucose, 0.5% w/v lactose, 1% w/v galactose, 2 mM M gSO 4). Cultures were incubated at 37° C. for 6 hours and grown for another 18 hours at 30° C. Cell pellets were harvested by centrifugation at 4° C. and resuspended in 40 mL 1:1 PBS: Bacterial Protein Extraction Reagent (BPER, ThermoFisher Scientific). Cell pellets were lysed by incubating in 60° C. water bath for 30 minutes. The resulting lysate was centrifuged at 8,000×g for 15 minutes at 4° C. The supernatant was filtered with 0.2 μm syringe filters and loaded onto a HiTrap Protein A HP column (Cytiva). The column was washed with PBS and protein was eluted with 100 mM acetic acid and immediately neutralized. Fractions were analyzed by Coomassie-stained SDS-PAGE. Fractions containing the correct protein molecular weight were pooled, concentrated, and flash frozen in liquid nitrogen. Protein concentrations were determined using absorbance at 280 nm and the protein extinction coefficient as calculated using ProtParam.

Subtiligase
(SEQ ID NO: 84)
MRGKKVWISLLFALALIFTMAFGSTSSAQAAGKSNGEKKYIVGFK
QTMSTMSAAKKKDVISEKGGKVQKQFKYVDAASATLNEKAVKELK
KDPSVAYVEEDHVAHAYAQSVPYGVSQIKAPALHSQGYTGSNVKV
AVIDSGIDSSHPDLKVAGGASFVPSETNPFQDNNSHGTHVAGTVA
ALDNSIGVLGVAPSASLYAVKVLGADGSGQYSWIISGIEWAIANN
MDVINLALGGPSGSAALKAAVDKAVASGVVVVAAAGNEGTSGSSS
TVGYPGKYPSVIAVGAVDSSNQRASFSSVGPELDVMAPGVSIQST
LPGNRYGAYSGTCMASAHVAGAAALILSKHPNWTNTQVRSSLENT
TTKLGDSFYYGKGLINVQAAAQLEHHHHHH

The italicized portion of the protein is removed by autoproteolysis. ESI-MS data for the purified protein are shown in FIG. 30F.

B. subtilis BG2864 cell were transformed with pB S42-pre-pro-stabiligase-His6 as described previously⁵¹.2xYT starter cultures (15 mL, 12.5 μg/mL chloramphenicol) were grown overnight and used to inoculate 200 mL 2xYT cultures (12.5 μg/mL chloramphenicol, 5 mM CaCl₂)) to OD 600 0.03-0.05. Cultures were incubated at 37° C. with vigorous shaking for 24 hours. Cells were pelleted by centrifugation at 4° C., and the resulting supernatant was added to 3 volumes of ice-cold ethanol. The precipitate was harvested by centrifugation at 4° C. and resuspended in wash buffer (50 mM sodium phosphate, 300 mM NaCl, 20 mM imidazole, pH 8.0). After brief centrifugation, Ni-NTA resin (400 mL) was added to the resuspended pellet and His-tagged protein was allowed to bind for 1 hour at 4° C. with gentle rocking. The Ni-NTA resin was collected by centrifugation at 500×g for 5 min, transferred to a 1 mL spin column, and washed with 4 mL wash buffer. Protein was eluted with 0.8 mL elution buffer (50 mM sodium phosphate, 300 mM NaCl, 250 mM imidazole, pH 8.0). The resulting protein was then concentrated using Amicon centrifugal filter units (10,000 MWCO) and further purified by size-exclusion chromatography (Superdex Hiload 75, GE Healthcare) using storage buffer (100 mM bicine, 5 mM DTT, pH 8.5). Collected fractions were analyzed by Coomassie-stained SDS-PAGE. Fractions containing the correct protein molecular weight were pooled, concentrated, and flash frozen in liquid nitrogen. Protein concentrations were determined using absorbance at 280 nm and the protein extinction coefficient as calculated using ProtParam.

eSrtA
(SEQ ID NO: 85)
MQAKPQIPKDKSKVAGYIEIPDADIKEPVYPGPATREQLNRGVSF
AEENESLDDQNISIAGHTFIDRPNYQFTNLKAAKKGSMVYFKVGN
ETRKYKMTSIRNVKPTAVEVLDEQKGKDKQLTLITCDDYNEETGV
WETRKIFVATEVKLEHHHHHH

ESI-M S data for the purified protein are shown in FIG. 30G.

Chemically competent E. coli BL21 (DE3) cells were transformed with pET 29a-eSrta and plated on LB-agar containing kanamycin (50 μg/mL). A starter culture was prepared by inoculating L B media (10 mL) containing kanamycin (50 μg/mL) with a single colony. The culture was grown overnight at 37° C. with shaking at 200 rpm. The starter culture was used to inoculate 1 L LB media in a baffled flask for overexpression. The culture was incubated at 37° C. with shaking at 200 rpm until OD 600 reached ˜0.6. IPTG was then added to a final concentration of 0.4 mM and the culture was incubate for an additional 3 h at 30° C. for protein expression. Cultures were centrifuged at 4,000×g at 4° C. for 20 min to pellet the cells. Cells were resuspended in 50 mL lysis buffer (50 mM Tris pH 8.0, 300 mM NaCl supplemented with 100 mM PM SF and Roche Complete EDTA-free Protease Inhibitor) and were lysed by sonication using a Q Sonica 700 probe sonicator (50% amplitude, 5 s on/5 s off, 15 cycles). Cell debris was pelleted by centrifugation at 10,000×g for 20 minutes at 4° C. The resultant supernatant was added to a conical tube containing 1 mL of Qiagen Ni-NTA agarose and the mixture was incubated for 1 hour at 4° C. with gentle rocking. The Ni-NTA resin was collected by centrifugation at 500×g for 5 min, transferred to a 15 mL column, and washed with 10 mL wash buffer (50 mM Tris pH 8.0, 300 mM NaCl, 20 mM imidazole). Protein was eluted with 5 mL elution buffer (50 mM Tris pH 8.0, 300 mM NaCl, 250 mM imidazole) and dialyzed against storage buffer (25 mM Tris pH 7.5, 150 mM NaCl) overnight at 4° C. The dialyzed protein was then concentrated using Amicon centrifugal filter units (10,000 MWCO). Protein concentrations were determined using absorbance at 280 nm and the protein extinction coefficient as calculated using ProtParam. Single-use aliquots were flash frozen in liquid nitrogen and stored at −80° C. until further use.

Sequence similarity network construction. A sequence similarity network (SSN) for representative members of the E1-like/ThiF superfamily was constructed using the Enzyme Function Initiative Enzyme Similarity Tool (Zallot et al. “The EFI Web Resource for genomic enzymology tools: leveraging protein, genome, and metagenome databases to discover novel enzymes and metabolic pathways.” Biochemistry 58, 4169-4182, 2019; Oberg et al. “EFI-EST, EFI-GNT, and EFI-CGFP: Enzyme Function Initiative (EFI) Web Resource for genomic enzymology tools.” J. Mol. Biol. 435, 168018, 2023). Input sequences consisted of those retrieved from the ThiF family in Pfam (Mistry et al. “The protein families database in 2021.” Nucleic Acids Res. 49, D412-D419, 2020) and were comprised of reviewed Uniprot entries (Consortium et al. “UniProt: the Universal Protein Knowledgebase in 2023.” Nucleic Acids Res 51, D523-D531, 2022) and M ccB sequences with annotated M ccA substrates from a bioinformatic analysis of M ccB homologs involved in biosynthesis of microcin C (Bantysh et al. “Enzymatic synthesis of bioinformatically predicted microcin C-like compounds encoded by diverse bacteria.” mBio 5, e01059-14, 2014). The SSN was visualized in Cytoscape 3.9.1 using a minimum alignment score of 100.

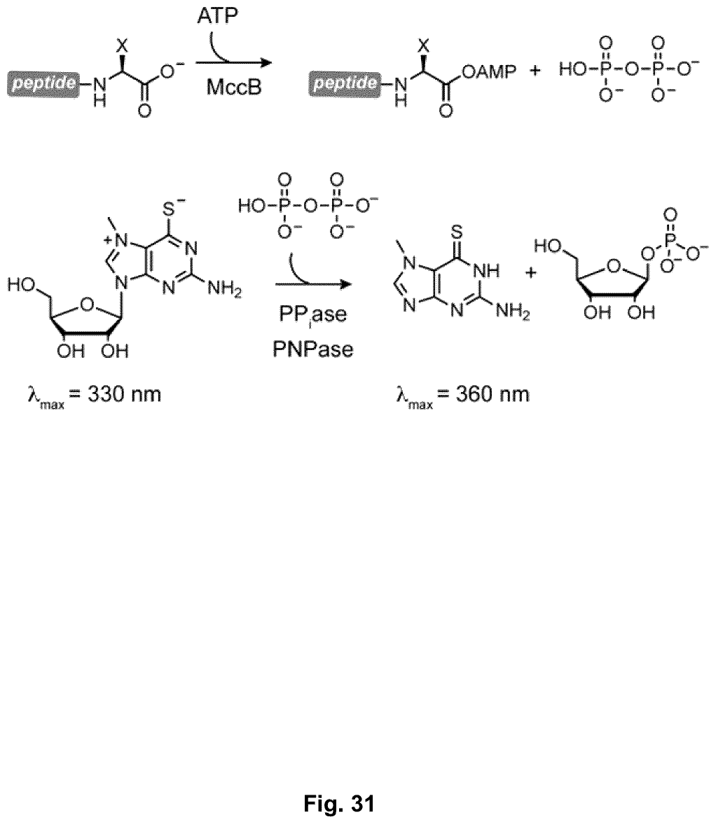

Purine nucleoside phosphorylase (PNP)/inorganic pyrophosphate (IP)-coupled kinetic assays. The kinetics of M ccB-catalyzed adenylation were characterized using an enzyme-coupled assay for detecting formation of pyrophosphate EnzCheck Pyrophosphate Assay Kit (Thermo Fisher Scientific) (FIG. 31). To screen M ccB for adenylation activity with C-terminal variants of MccA, 5 μM of the appropriate MccB variant was incubated with 0.25 mM of each M ccA variant in a reaction with 0.25 mM ATP, 5 mM M gCl2, 0.5 U/mL purine nucleoside phosphorylase (PNP), and 0.5 U/mL inorganic pyrophosphatase (IP). To screen E. coli, H. pylori, and L. johnsonii M ccB homologs for adenylation activity with MccA homologs, 5 μM of the appropriate MccB enzyme was incubated with 0.25 mM of each M ccA variant in a reaction with 0.25 mM ATP, 5 mM M gCI2, 0.5 U/mL purine nucleoside phosphorylase (PNP), and 0.5 U/mL inorganic pyrophosphatase (IP). Reactions containing HpM ccA-N7G and LjM ccA-N 7G peptides also contained 5 mM M esna and 5 mM TCEP. To collect steady-state kinetics data for individual peptide substrates, the peptide concentration was varied from 0-600 μM in a reaction with 5 μM MccB, 0.25 mM ATP, 5 mM MgCl2, 0.5 U/mL purine nucleoside phosphorylase (PNP), and 0.5 U/mL inorganic pyrophosphatase (IP). Initial fitting of kinetic data for the M ccA-N 7G substrate revealed that the apparent KM (8.9+0.9 μM) for MccA-N7G was close to the concentration of MccB (5 μM) used in the reaction, invalidating the assumption that the concentration of free substrate unbound to enzyme is approximately equal to the total substrate concentration (the free ligand approximation) that is used to derive the Michaelis-Menten equation. We therefore repeated measurements for the MccA-N7G peptide using an MccB concentration of 0.5 M and a substrate concentration range from 0-100 μM. Reactions were initiated with the addition of M ccB and absorbance at 360 nm was monitored using a Tecan Infinite M 200 plate reader. Initial rates of pyrophosphate production were calculated based on the initial rate of absorbance change and the resulting purine analog extinction coefficient (11,000 M-1 cm-1). Reactions were carried out in triplicate and data analysis was performed using GraphPad Prism 10.

LC-TOF MS analysis of M ccB reactions with synthetic peptide substrates. For analysis of formation of N-AM Pylated M ccA, MccA substrate (250 μM) was incubated with 5 μM MccB and 5 mM ATP in reaction buffer (75 mM Tris pH 8.0, 5 mM MgCl2). Reactions were incubated for 16 hours at room temperature and quenched with addition of an equivalent volume of 0.6% TFA. These reaction conditions were used for M ccBs from E. coli, H. pylori, L. johnsonii, and H. somni. Quenched reactions were centrifuged at 8,000×g for 10 minutes and analyzed using LC-TOF MS as described in Mass spectrometry.

For analysis of E. coli M ccB-catalyzed thioesterification, MccA-N7G substrate (250 μM) was incubated with 5 μM MccB, 5 mM ATP, 5 mM Mesna, and 5 mM TCEP in reaction buffer (75 mM Tris pH 8.0, 5 mM M gCl2). Reactions were incubated for 16 hours at room temperature and quenched with addition of an equivalent volume of 0.6% TFA. To screen the ability of E. coli MccB, H. pylori MccB, L. johnsonii MccB to catalyze thioesterification of M ccA-N7Gs derived from each species, reactions contained 5 μM M ccB, 5 mM ATP, 50 mM M esna, and 12.5 mM TCEP in reaction buffer (75 mM Tris pH 8.0, 5 mM M gCl2). To screen the ability of H. somni M ccB to catalyze thioesterification of HsLACE peptides, reactions contained M ccB (5-25 mM), ATP (5 mM), M gCl2 (5 mM), peptide substrate (0.25 mM), 25 mM M esna or AcCysNHM e, and reaction buffer (100 mM HEPES, pH 8.0). Quenched reactions were centrifuged at 8,000×g for 10 minutes and analyzed using LC-TOF MS as described in Mass spectrometry.

Screening nucleophiles for MccB-mediated ligation of synthetic peptide substrates. Nucleophile were dissolved in water and adjusted to neutral pH using paper pH strips (EM D Millipore). Solutions were stored at −80° C. until further use, unless otherwise noted. M ccA-N7G (250 μM) was incubated with 5 μM MccB, 5 mM ATP, and the corresponding concentration of nucleophile in reaction buffer (25 mM Tris pH 8.0, 10 mM M gCl2). Reactions were incubated for 16 hours at room temperature and quenched with addition of an equivalent volume of 0.6% TFA. Quenched reactions were centrifuged at 8,000×g for 10 minutes and analyzed using LC-TOF MS as described in Mass spectrometry. Extracted ion chromatogram (EIC) peak areas were calculated using Agilent TOF Quantitative Analysis v11.0. Percent conversion of substrate to nucleophile-modified product was calculated using the integrated peak areas from the substrate peptide EIC and production EIC using the formula shown below.

$$percent conversion = \frac{product peak area}{product peak area + s u b s trate peak area} \times 100$$

Raw data are available in the Dryad repository at DOI: 10.5061/dryad.c59zw 3rkb.

M ccB-mediated C-terminal ligation of M ccA-N7G with Cys. M ccA-N 7G (250 μM) was incubated for 16 hours at room temperature in reactions containing 5 μM MccB, 5 mM ATP, and the appropriate concentration of Cys/TCEP in reaction buffer (25 mM Tris pH 8.0, 10 mM M gCl2). For maleimide functionalization, reactions were performed with 1 mM Cys/TCEP for 16 hours followed by a 1-hour incubation with 1.5 mM maleimide. All reactions were quenched, centrifuged at 8,000×g for 10 minutes, and analyzed using LC-TOF MS as described in Mass spectrometry.

MccA-N7G thioester exchange with N-terminal Cys peptide. C-terminal thioester was prepared in reactions containing 250 μM MccA-N7G, 5 μM MccB, 5 mM ATP, and 5 mM Mesna/TCEP in reaction buffer (25 mM Tris pH 8.0, 10 mM M gCl2). A fter incubating for 16 hours at room temperature, 20 mM Cys-Trp peptide was added to each reaction and incubated for an additional 4 hours. Reactions were quenched with the addition of an equivalent volume of 0.6% TFA. Quenched reactions were centrifuged at 8000×g for 10 minutes and analyzed using LC-TOF MS as described in Mass spectrometry.

M ccB-mediated ligation of GFP-MccA-N7G protein substrates. GFP-M ccA-N7G variant (50 μM) was incubated with 5 μM MccB, 5 mM ATP, and the appropriate concentration of cysteine or Mesna/TCEP in reaction buffer (25 mM Tris pH 8.0, 10 mM M gCl2). Reactions were incubated at room temperature for the indicated times. For labeling time course experiments, reactions were quenched with the addition of an equivalent volume of 0.6% TFA. For maleimide functionalization of Cys-ligated proteins, reactions were desalted using 75 μL 7K MWCO Zeba Micro Spin Desalting Columns (Thermo Fisher Scientific) and incubated with maleimides overnight at 4° C. Reactions were centrifuged at 8,000×g for 10 minutes and analyzed using LC-TOF MS as described in Mass spectrometry.

GFP-MccA-N7G thioester exchange time course with Cys-Trp dipeptide. C-terminal thioester was prepared in reactions containing 50 μM cysteine-free GFP-M ccA-N7G, 5 μM MccB, 5 mM ATP, and 5 mM Mesna/TCEP in reaction buffer (25 mM Tris pH 8.0, 10 mM M gCl2). After incubating for 1 h at room temperature reactions were desalted using 75 μL 7K MWCO Zeba Micro Spin Desalting Columns (Thermo Fisher Scientific). Cys-Trp was added to a final concentration of 5 mM. Reactions were quenched at various time points by addition of an equivalent volume of 0.6% TFA. Quenched reactions were centrifuged at 8,000×g for 10 minutes and analyzed using LC-TOF MS as described in Mass spectrometry.

GFP-MccA-N7G expressed protein ligation with N-terminal Cys peptide followed by copper-free click chemistry. Reactions contained 50 μM GFP-M ccA-N 7G, 5 μM MccB, 5 mM ATP, 5 mM M esna/DTT, and 5 mM CGAGS-azidoA la peptide (SEQ ID NO: 56) in reaction buffer (25 mM Tris pH 8.0, 10 mM M gCl2). After incubating for 4 h at room temperature, reactions were desalted using 75 μL 7K MWCO Zeba Micro Spin Desalting Columns (Thermo Fisher Scientific). Desalting was repeated twice more. Dibenzocyclooctyne (DBCO)-PEG4-biotin was added to a final concentration of 3 mM and incubated for 1 h at room temperature. Reactions were desalted once more and DTT was added to a final concentration of 5 mM for a 30-minute incubation at room temperature. Reactions were centrifuged at 8,000×g for 10 minutes and analyzed using LC-TOF MS as described in Mass spectrometry.

LC-TOF MS analysis of M ccB-catalyzed N-AM Pylation of M ccA positional scanning peptide library. MccA variants were synthesized individually in-house (according to Solid-phase peptide synthesis) or were purchased from Peptide2.0. For analysis of formation of N-AMPylated MccA variants, MccA substrate (250 μM) was incubated with 5 μM MccB and 5 mM ATP in reaction buffer (75 mM Tris pH 8.0, 5 mM M gCl2). Reactions were incubated for 16 hours at room temperature and quenched with addition of an equivalent volume of 0.6% TFA. Quenched reactions were centrifuged at 8,000×g for 10 minutes and analyzed using LC-TOF MS as described in Mass spectrometry. Percent conversion was calculated using peak areas from substrate and product EICs. Raw data are available in the Dryad repository at DOI: 10.5061/dryad.c59zw3rkb.

M ccB homolog reactions with GFP-TeCH-tag fusion proteins. E. coli, H. pylori, and L. johnsonii were screened for protein thioesterification using GFP fused with the M ccA-N7G homolog sequences. Reactions contained 5 μM MccB, 5 mM ATP, 50 μM GFP-TeCH fusion, 50 mM M esna, and 12.5 mM TCEP in reaction buffer (75 mM Tris-HCl pH 8.0, 5 mM M gCl2). For multiplexed reactions containing all three GFP-TeCH fusions, each GFP-TeCH fusion was added to a final concentration of 16 μM. Reactions were quenched after 16 hours at room temperature by adding two volumes of 0.6% TFA. Samples were analyzed using LC-TOF MS as described in Mass spectrometry.

M ccB/subtiligase-catalyzed ligation of protein substrates. M ccB/subtiligase-catalyzed C-terminal amide bond formation was performed in one-pot reactions containing 50 μM TeCH-tagged protein, 5 μM MccB, 5 mM ATP, 5 UM subtiligase, 5 mM peptide substrate, and 5 mM M esna/DTT in reaction buffer (25 mM Tris pH 8.0, 10 mM M gCl2). After incubating for appropriate time at room temperature, reactions were diluted in 75 mM HEPES pH 8.0 and analyzed using LC-TOF MS as described in Mass spectrometry.

M ccB/subtiligase-mediated ligation of anti-GFP rAb followed by copper free click chemistry. C-terminal bioconjugation was performed in one-pot reactions containing 50 μM protein, 5 μM MccB, 5 mM ATP, 5 μM subtiligase, 5 mM peptide substrate, and 5 mM M esna in reaction buffer (75 mM Tris pH 8.0, 5 mM M gCl2). After incubating for the appropriate time at room temperature, reactions were twice buffer exchanged into 75 mM HEPES, pH 8.0 using 500 μL 7K MWCO Zeba Micro Spin Desalting Columns (Thermo Fisher Scientific), and analyzed using LC-TOF MS. Absorbance at 280 nm was used to estimate the resulting protein concentration, and 2-5 molar equivalents of the DBCO reagent were added to each sample. After 4 hours at room temperature, the samples were twice buffer exchanged as before, and analyzed using LC-TOF MS as described in Mass spectrometry.

Cell culture and immunofluorescence. Flp-In T-Rex 293T cells (Thermo Fisher Scientific) were grown in DM EM supplemented with 10% fetal bovine serum, 100 U/mL penicillin, 100 mg/mL streptomycin, and other antibiotics as appropriate. Cells were tested every six months for mycoplasma contamination using the LookOut Mycoplasma PCR Detection Kit (Sigma-Aldrich) according to the manufacturer's instructions. A stable cell line expressing IgK-eGFP-PDGF receptor B-chain transmembrane domain (PDGFRTM; SEQ ID NO: 86) under a doxycycline-inducible promoter was introduced by transfecting cells with pcDNA5/FRT/TO-IgK-eGFP-PDGFRTM according to the manufacturer's instructions. This construct included an N-terminal Igk signal peptide and a C-terminal PDGF receptor b-chain transmembrane domain to target eGFP to the cell surface. For immunofluorescence experiments, cells were seeded at 10,000 cells per well in a 96-well plate and grown to 50% confluency. Doxycycline (1 μg/mL) was then added to induce cell surface GFP expression. After 20 hours, cells were washed three times with ice cold PBS, fixed with PBS+4% paraformaldehyde for 10 minutes, and washed three times with PBS +3% BSA. Cells were stained with 10 μg/mL aGFP-rAb in PBS+3% BSA for 1 hour at room temperature and washed three times with PBS+3% BSA. Imaging was performed on an Echo Revolve epifluorescence microscope in the inverted configuration.

IgK-GFP-PDGFR-TM sequence. The N-terminal IgK leader sequence is underlined, a V 5 tag is shown in italic, and the C-terminal PDGF receptor transmembrane domain is underlined.

(SEQ ID NO: 87)
METDTLLLWVLLLWVPGSTGGKPIPNPLLGLDSTGSGGGASMVSK
GEELFTGVVPILVELDGDVNGHKFSVSGEGEGDATYGKLTLKFIC
TTGKLPVPWPTLVTTLTYGVQCFSRYPDHMKQHDFFKSAMPEGYV
QERTIFFKDDGNYKTRAEVKFEGDTLVNRIELKGIDFKEDGNILG
HKLEYNYNSHNVYIMADKQKNGIKVNFKIRHNIEDGSVQLADHYQ
QNTPIGDGPVLLPDNHYLSTQSALSKDPNEKRDHMVLLEFVTAAG
ITLGMDELYKGSGGSGGGGSAVGODTQEVIVVPHSLPFKVVVISA
ILALVVLTIISLIILIMLWQKKPR

C-terminal labeling using eSrtA. GFP-LPETGG (50 mM) was incubated with eSrtA (2.5 mM) and a triglycine peptide (GGG, variable concentration) in reaction buffer (50 mM Tris-HCl, pH 7.5, 150 mM NaCl, 10 mM CaCl₂)). Reactions were incubated at room temperature for 30 minutes and then analyzed by LC-TOF MS as described in Mass spectrometry. Percent conversion of GFP-LPETGG (27,814 Da) to cyclized GFP (27,681 Da) or ligated GFP (27,871 Da) was calculated using peak areas from the deconvoluted mass spectra.

Dual N- and C-terminal labeling with eSrtA and M ccB/subtiligase. For one-pot reactions, Gly-Ser-MBP-TeCH (25 mM) was incubated with M ccB (5 mM), subtiligase (5 mM), and AFAGAGS-azidoA la (5 mM, M ccB/subtiligase substrate), 5 mM ATP, eSrtA (2.5 mM), and 5-FAM-LPETGG (2 mM) in reaction buffer (100 mM HEPES, pH 8.0, 150 mM NaCl, 10 mM CaCl₂), 5 mM M gCl2) for 12 h. For telescoping reactions, Gly-Ser-MBP-TeCH (25 mM) was incubated with M ccB (5 mM), subtiligase (5 mM), and AFAGAGS-azidoAla (5 mM, MccB/subtiligase substrate) in reaction buffer for 4 h at room temperature, followed by addition of eSrtA (2.5 mM) and 5-FAM-LPETGG (2 mM) and incubation for an additional 4 h at room temperature.

Chemical synthesis of LRLRGG-Mes thioester peptide. LRLRGG-hydrazide was purchased from GenScript. The hydrazide peptide was converted to the M es thioester as previously described (49, 71). LRLRGG-hydrazine (4 mg, 6 mmol, 1 equivalent) was dissolved in 200 mM sodium phosphate, 6 M guanidinium hydrochloride (GdnHCI, 0.5 mL). The solution was cooled with stirring to −15° C. in a bath of aqueous saturated sodium chloride and ice. A freshly prepared aqueous solution of sodium nitrite (0.5 M, 0.12 mL, 60 mmol, 10 equivalents) was added and the reaction was allowed to proceed for 20 min. A solution of sodium mercaptoethanesulfonate (Mesna, 29.6 mg, 180 mmol, 30 equivalents) in 200 mM sodium phosphate, pH 7.0, 6 M GdnHCI (0.72 mL) was added to the reaction. The mixture was adjusted to pH 7 using 1 N NaOH and reaction was allowed to proceed for 2 h at ambient temperature. The reaction mixture was purified using a semi-preparative ZORBAX Eclipse XDB-C18 column (9.4× 250 mm, 5 μm) coupled to an Agilent 1260 Infinity II HPLC. A 90-min gradient from 100% mobile phase A (0.1% trifluoroacetic acid in water) to 100% B (acetonitrile) at 2 mL/min was used to separate the desired product. Fractions were analyzed by LC-TOF MS and those containing the pure product were pooled and lyophilized. The resultant product was dissolved in water and quantified using the Pierce Quantitative Fluorometric Peptide Assay Kit (Thermo Fisher Scientific). The yield was 80 mL of a 34 mM solution (2.2 mg, 2.7 mmol, 45%).

H. somni M ccB-catalyzed peptide thioester synthesis. For screening the activity of HsM ccB on HsTeCH, HsLACE1-4, and LACE substrate peptide, 20 mL reactions were prepared that contained 100 mM HEPES, pH 8.0, HsM ccB (5-25 mM), HsTeCH, HsLACE1-4, or LACE peptide (250 mM), ATP (5 mM), M gCl2 (5 mM), and thiol (M esna or AcCysNHMe, 25 mM). Reactions were initiated by addition of HsM ccB and incubated for 16-20 h at room temperature. Raw data are available in the Dryad repository at DOI: 10.5061/dryad.c59zw3rkb.

For larger scale synthesis of MLGLRGG-Mes (HsLACE3-M es) (SEQ ID NO: 88), identical reaction conditions and reagent concentrations were used in a volume of 200 mL. Reactions were quenched by addition of trifluoroacetic acid (TFA) to 1% final concentration and were desalted on SOLA HRP C18 solid-phase extraction columns (10 mg format, Thermo Fisher Scientific). Columns were conditioned with 100% acetonitrile (500 mL) and equilibrated with 0.1% TFA (2× 1 mL). The acidified sample was then loaded onto the column. The column was washed with 0.1% TFA (2×1 mL) and eluted with 80% acetonitrile/20% water (2×150 mL). The eluted peptide thioester was dried in a vacuum concentrator (SpeedV ac SPD 130DLX, Thermo Fisher Scientific) and dissolved in water. The recovered peptide was quantified using the Pierce Quantitative Fluorometric Peptide A ssay Kit (Thermo Fisher Scientific). Typical recovery from desalting was 50%.

Lysine acylation using conjugating enzymes (LACE) of GFP-LACE-tag using M ccB-generated thioesters. GFP-LACE-tag (15 mM), U bc9 (60 mM), MLGLRGG-Mes (SEQ ID NO: 88) or other C-terminal thioesters as indicated in the text (150 mM-1500 mM) were incubated in reaction buffer (100 mM HEPES, pH 8.0, 50 mM K CI) at 30° C. for 16 h. For pH optimization, reaction buffer was either 100 mM HEPES, pH 7.6, 50 mM KCl or 100 mM HEPES, pH 8.0, 50 mM KCl. For analyzing the effect of thiol concentration on reaction efficiency, Mesna (0-25 mM) was included in the reaction. Optimal reaction conditions for transfer of MLGLRGG-Mes (SEQ ID NO: 88) to GFP LACE used 100 mM HEPES, pH 8.0, 50 mM KCl and omitted thiol.

What is claimed is:

1. A polypeptide fusion comprising a polypeptide having a C-terminus and a peptide tag fused to the C-terminus of the polypeptide,
wherein the peptide tag comprises a sequence of formula (X)ₙX′, wherein X is any amino acid, n is an integer from 6 to 55, and X′ is an amino acid other than asparagine, and
wherein the polypeptide is not fused to the peptide tag in nature.

2. The polypeptide fusion of claim 1, wherein X′ is selected from alanine, glycine, serine, and threonine.

3. The polypeptide fusion of claim 1, wherein the peptide tag is derived from a peptide selected from SEQ ID NOs: 5-51, wherein the C-terminal asparagine of the peptide is replaced with X′.

4. The polypeptide fusion of claim 1, wherein the peptide tag is selected from SEQ ID NOs: 52-55.

5. A method of modifying a C-terminus of a polypeptide, comprising
providing the polypeptide, wherein the polypeptide is a substrate of an E1-like superfamily enzyme;
reacting the polypeptide, the E1-like superfamily enzyme, and ATP under conditions to O-A M Pylate the C-terminus of the polypeptide; and
reacting the C-terminally O-AM Pylated polypeptide with a nucleophile comprising a functional group to provide a modified polypeptide comprising the C-terminal functional group.

6. The method of claim 5, wherein the polypeptide comprises a C-terminal carboxylate.

7. The method of claim 5, wherein the polypeptide is the polypeptide fusion of claim 1.

8. The method of claim 5, wherein the nucleophile is a thiol, a hydrazine, an alkoxyamine, a hydroxylamine, an amine, or an alcohol.

9. The method of claim 8, wherein the nucleophile is a thiol nucleophile and the C-terminal functional group is a thioester.

10. The method of claim 9, wherein the thiol nucleophile is N-acetyl-L-cysteine, N-acetylcysteamine, sodium 2-mercaptoethane sulfonate (M esna), dithiothreitol (DTT), or L-cysteine (Cys).

11. The method of claim 9, further comprising transthiolating the C-terminal thioester polypeptide.

12. The method of claim 11, wherein the C-terminal thioester polypeptide is transthiolated with dithiothreitol (DTT), betamercaptoethanol (BM E), or 4-mercaptophenylacetic acid (M PAA).

13. The method of claim 5, further comprising reacting the C-terminal functional group with a bioconjugation agent.

14. The method of claim 13, wherein the C-terminal functional group is a thioester, and the bioconjugation agent comprises an azide, a malemide, a para-fluoro compound, an alkene, an alkyne, or a vinyl sulfone.

15. The method of claim 13, wherein the bioconjugation agent further comprises a cargo molecule.

16. The method of claim 13, further comprising reacting the bioconjugation agent with a reactive molecule comprising a cargo molecule to provide a fusion polypeptide labeled with the cargo molecule.

17. The method of claim 15, wherein the cargo molecule comprises biotin, an imaging agent, a pharmaceutical agent, a nanoparticle, a radiolabel, a polymer, or an amino acid.

18. The method of claim 8, further comprising exchanging the thioester with reactive moiety comprising an N-terminal cysteine to form a peptide bond.

19. The method of claim 8, further comprising reacting the C-terminal thioester-modified polypeptide with a target peptide in the presence of a ligase to provide a target peptide fused to the C-terminus of the polypeptide.

20. The method of claim 5, wherein the E1-like superfamily enzyme is selected from the group consisting of Escherichia coli M ccB (SEQ ID NO: 1), Helicobacter pylori M ccB (SEQ ID NO: 2), Lactobacillus johnsonii M ccB (SEQ ID NO: 3), and Histophilus somni M ccB (SEQ ID NO: 4), or a polypeptide with at least 90%, 91%, 92%, 93%, 94%, 95%, 96%, 97%, 98%, or 99% sequence identity to any of the foregoing enzymes.


SEQUENCE LISTING (92 sequences)

SEQ ID NO: 1 - protein, 350 aa, Escherichia coli
MDYILGRYVKIARYGSGGLVGGGGKEQYVEDLALWENIIKTAYCFITPSSYTAALETVNIPEKDFSNCFRFLKENFFIIPSEYNNSTENNRYSRNFLHYQSYGANPVLVQDKLKDAKVVILGCGGIGNHVSVILATSGIGEIILIDNDQIENTNLTRQVLFSENDVGKNKTEVIKRELLKRNSEISVSEIALNINDYTDLHKVPEADIWVVSADHPFNLINWVNKYCVRANQPYINAGYVNDIAVFGPLYVPGKTGCYECQKVVADLYGSEKENIDHKIKLINSRFKPATFAPVNNVAAALCAADVIKFIGKYSEPLSLNKRIGIWSDEIKIHSQNMGRSPVCSVCGNRM

SEQ ID NO: 2 - protein, 280 aa, Helicobacter pylori
MQWYQTSFSACVGQTDTENIIGLGTYQYCVDHNEFEKSLKLLVFLRMKKRMAEIKSFMETSKIEHNIFDKLVANKLITSFILNPNDEQNFKNHLFIDLMSSKPELTIDNFKRTIFIIIGCGGIGNFVSYALASFYPKKLILLDKDTVDFSNLNRQFLFDKNYISQYKTSAIKQALSSRFSINIETVDDFASEDNLEEIFSKHKKENLFGIVSGDNPNTVQLATRFFCKCRIPFLNIGYLNDISLIGPFYIPSLSCCPFCHNSFALDDKKDGDENLDICLI

SEQ ID NO: 3 - protein, 354 aa, Lactobacillus johnsonii
MFYKTSYLATGGCSNHQGILGVGTKQYFVSEADYLKSLKILDFLLNKKTYDEVIKFCEKNNINKSIFDTLVEHNLIVKENLYVEKKDDLNFKNKLYFHALGLNGNALAKEFADTTFVIVGCGGIGNFISFAIGSLSPRKIELIDGDKIEKSNLNRQFLFTENDIGKYKVDVLKKNLVERNNKLSISEYKEYVSKEVLHNIFEQNKKNKTLVILSGDSFSALSLTAKACVKSEIPFLNIGYLNDISAIGPFYIPGISSCPFCHNALSISDDISSGHNESKILEDRINANNEAPSSFTNNALAASMGIADIIEFLSHNYERINSLNKRFGINSATFEKYVLEVNRDRKCEICSHGE

SEQ ID NO: 4 - protein, 347 aa, Histophilus somni
MKYITSKHVFFDYLNENEFVIGIGSNQEITNNKDYFNNCLNLCYFCINPKSISEILSFIKDNNIDILYFDKMKKMKFITKEIIDFNDRYSRNHLYYNALGYKIYDIQNKISKSHILIVGAGGIGNICSYLLGTIGIKKLSIIDDDIVEESNLNRQFLFREKDINKNKVETIKRELLSIRKDIIIDIFPEKLNKSILDKISQIDLVICSADDEYCIDMINEFCCFNKIPLINVGYLNDISVIGPFYIPKLEYSCCLCCDKSIYLENDVIDEKVKKIKSVTKAPSTIINNFFAGAMLGSELIKFFARDYKSMQSINSVIGIHNKNFKYEEIKLAKNYNCKYCGVNNETL

SEQ ID NO: 5 - protein, 19 aa, Arthrobacter sp.
MLKLKRLAKRRSKLNFVFN

SEQ ID NO: 6 - protein, 19 aa, Arthrobacter sp.
VLKLKRLTKRRSKLNFVFN

SEQ ID NO: 7 - protein, 19 aa, Arthrobacter sp.
VLKLKRLAKRRSKLNFVFN

SEQ ID NO: 8 - protein, 19 aa, Bacillus amyloliquefaciens
MLKIRKVKIVRAQNGHYTN

SEQ ID NO: 9 - protein, 7 aa, Bartonella alsatica
MDHIGFN

SEQ ID NO: 10 - protein, 7 aa, Bartonella henselae
MDHPEFN

SEQ ID NO: 11 - protein, 7 aa, Bartonella vinsonii
MDHRGFN

SEQ ID NO: 12 - protein, 7 aa, Borrelia coriaceae
MRGKTLN

SEQ ID NO: 13 - protein, 7 aa, Borreliella garinii
MLGKNCN

SEQ ID NO: 14 - protein, 7 aa, Brenneria salicis
MDHSMNN

SEQ ID NO: 15 - protein, 7 aa, Chlamydiales bacterium
MDTVTTN

SEQ ID NO: 16 - protein, 7 aa, Clostridium paradoxum
MRLIIRN

SEQ ID NO: 17 - protein, 7 aa, Dickeya zeae
MISVSSN

SEQ ID NO: 18 - protein, 7 aa, Escherichia coli
MRTGNAN

SEQ ID NO: 19 - protein, 7 aa, Gardnerella vaginalis
MHVTPSN

SEQ ID NO: 20 - protein, 7 aa, Glaesserella parasuis
MRGRRVN

SEQ ID NO: 21 - protein, 7 aa, Helicobacter pylori
MKLSYRN

SEQ ID NO: 22 - protein, 7 aa, Histophilus somni
MRGRRLN

SEQ ID NO: 23 - protein, 7 aa, Lactobacillus johnsonii
MHRIMKN

SEQ ID NO: 24 - protein, 7 aa, Lactobacillus murinus
MSLMIKN

SEQ ID NO: 25 - protein, 7 aa, Marinomonas sp.
MIARLLN

SEQ ID NO: 26 - protein, 7 aa, Moraxella caprae
MIAMAHN

SEQ ID NO: 27 - protein, 8 aa, Moraxella catarrhalis
MISINPFN

SEQ ID NO: 28 - protein, 7 aa, Murinocardiopsis flavida
MNGDGDN

SEQ ID NO: 29 - protein, 23 aa, Mycobacteroides abscessus
MKTLAVKIRRIVRAQGSQKSGSN

SEQ ID NO: 30 - protein, 7 aa, Nocardiopsis alkaliphila
MNDDGSN

SEQ ID NO: 31 - protein, 7 aa, Nocardiopsis dassonvillei
MNDDGNN

SEQ ID NO: 32 - protein, 7 aa, Nocardiopsis flavescens
VNDDGNN

SEQ ID NO: 33 - protein, 21 aa, Nocardiopsis sp.
MRRISMTPVRKVKGSTTVVAN

SEQ ID NO: 34 - protein, 7 aa, Pectobacterium parmentieri
MEHTTNN

SEQ ID NO: 35 - protein, 7 aa, Photorhabdus laumondii
MISSGSN

SEQ ID NO: 36 - protein, 7 aa, Photorhabdus temperata
MISIASN

SEQ ID NO: 37 - protein, 7 aa, Pilibacter termitis
MAGPLRN

SEQ ID NO: 38 - protein, 7 aa, Serratia symbiotica
MIGGLNN

SEQ ID NO: 39 - protein, 19 aa, Staphylococcus delphini
MLEIQEVKEIEGRSAHVSN

SEQ ID NO: 40 - protein, 19 aa, Staphylococcus gallinarum
MLEIKEVKEIEGRSKNLSN

SEQ ID NO: 41 - protein, 19 aa, Staphylococcus hominis
MLEIKEIKDVQANSVNVSN

SEQ ID NO: 42 - protein, 19 aa, Streptacidiphilus pinicola
MLKIVKKPVVKTDSIMVHN

SEQ ID NO: 43 - protein, 8 aa, Streptobacillus notomytis
VIFTVVKN

SEQ ID NO: 44 - protein, 7 aa, Streptococcus thermophilus
MKGTILN

SEQ ID NO: 45 - protein, 7 aa, Streptococcus azizii
MIGPIQN

SEQ ID NO: 46 - protein, 7 aa, Streptococcus suis
MVGPVQN

SEQ ID NO: 47 - protein, 56 aa, Synechococcus sp.
MTQPNDRQLSNEELSDVAAGLFRRTFFKPRTSRKTLLQPKRLDKVAKNQLWADMMN

SEQ ID NO: 48 - protein, 7 aa, Thermobifida halotolerans
MNGDGSN

SEQ ID NO: 49 - protein, 17 aa, Turneriella parva
MKIEKTAKKITRTGGAN

SEQ ID NO: 50 - protein, 42 aa, Yersinia pseudotuberculosis
MHQSEIKLTKRLKIKRVDVNKVKEQQKKVLECGAATCGGGSN

SEQ ID NO: 51 - protein, 7 aa, Yersinia wautersii
MDHTTSN

SEQ ID NO: 52 - protein, 7 aa, synthetic construct
MRTGNAG

SEQ ID NO: 53 - protein, 7 aa, synthetic construct
MKLSYRG

SEQ ID NO: 54 - protein, 7 aa, synthetic construct
MHRIMKG

SEQ ID NO: 55 - protein, 7 aa, synthetic construct
MRGRRLG

SEQ ID NO: 56 - protein, 6 aa, synthetic construct
  MOD_RES 6: note = 3-azido-L-alanine
CGAGSA

SEQ ID NO: 57 - protein, 8 aa, synthetic construct
  MOD_RES 8: note = Azidolysine
AFAGAGSK

SEQ ID NO: 58 - protein, 6 aa, synthetic construct
LPETGG

SEQ ID NO: 59 - protein, 8 aa, synthetic construct
  MOD_RES 8: note = 3-azido-L-alanine
AFAGAGSA

SEQ ID NO: 60 - protein, 6 aa, synthetic construct
  MOD_RES 1: note = 5-Carboxyfluorescein (5-FAM) attached
LPETGG

SEQ ID NO: 61 - protein, 6 aa, synthetic construct
LRLRGG

SEQ ID NO: 62 - protein, 7 aa, Histophilus somni
MRGRRGG

SEQ ID NO: 63 - protein, 7 aa, Histophilus somni
MRGLRGG

SEQ ID NO: 64 - protein, 7 aa, Histophilus somni
MLGLRGG

SEQ ID NO: 65 - protein, 7 aa, Histophilus somni
MLRLRGG

SEQ ID NO: 66 - protein, 6 aa, synthetic construct
  MOD_RES 6: note = Mesna thioesterification
LRLRGG

SEQ ID NO: 67 - protein, 5 aa, synthetic construct
LPXTG

SEQ ID NO: 68 - protein, 11 aa, synthetic construct
GGGSMRTGNAG

SEQ ID NO: 69 - protein, 373 aa, synthetic construct
  REGION 1..21: note = N-terminal His tag and TEV protease cleavage site sequence
MGHHHHHHDYDIPTTENLYFQGSMDYILGRYVKIARYGSGGLVGGGGKEQYVEDLALWENIIKTAYCFITPSSYTAALETVNIPEKDFSNCFRFLKENFFIIPSEYNNSTENNRYSRNFLHYQSYGANPVLVQDKLKDAKVVILGCGGIGNHVSVILATSGIGEIILIDNDQIENTNLTRQVLFSENDVGKNKTEVIKRELLKRNSEISVSEIALNINDYTDLHKVPEADIWVVSADHPFNLINWVNKYCVRANQPYINAGYVNDIAVFGPLYVPGKTGCYECQKVVADLYGSEKENIDHKIKLINSRFKPATFAPVNNVAAALCAADVIKFIGKYSEPLSLNKRIGIWSDEIKIHSQNMGRSPVCSVCGNRM

SEQ ID NO: 70 - protein, 303 aa, synthetic construct
  REGION 1..21: note = N-terminal His tag and TEV protease cleavage site sequence
MGHHHHHHDYDIPTTENLYFQGSMQWYQTSFSACVGQTDTENIIGLGTYQYCVDHNEFEKSLKLLVFLRMKKRMAEIKSFMETSKIEHNIFDKLVANKLITSFILNPNDEQNFKNHLFIDLMSSKPELTIDNFKRTIFIIIGCGGIGNFVSYALASFYPKKLILLDKDTVDFSNLNRQFLFDKNYISQYKTSAIKQALSSRFSINIETVDDFASEDNLEEIFSKHKKENLFGIVSGDNPNTVQLATRFFCKCRIPFLNIGYLNDISLIGPFYIPSLSCCPFCHNSFALDDKKDGDENLDICLI

SEQ ID NO: 71 - protein, 377 aa, synthetic construct
  REGION 1..21: note = N-terminal His tag and TEV protease cleavage site sequence
MGHHHHHHDYDIPTTENLYFQGSMFYKTSYLATGGCSNHQGILGVGTKQYFVSEADYLKSLKILDFLLNKKTYDEVIKFCEKNNINKSIFDTLVEHNLIVKENLYVEKKDDLNFKNKLYFHALGLNGNALAKEFADTTFVIVGCGGIGNFISFAIGSLSPRKIELIDGDKIEKSNLNRQFLFTENDIGKYKVDVLKKNLVERNNKLSISEYKEYVSKEVLHNIFEQNKKNKTLVILSGDSFSALSLTAKACVKSEIPFLNIGYLNDISAIGPFYIPGISSCPFCHNALSISDDISSGHNESKILEDRINANNEAPSSFTNNALAASMGIADIIEFLSHNYERINSLNKRFGINSATFEKYVLEVNRDRKCEICSHGE

SEQ ID NO: 72 - protein, 370 aa, synthetic construct
  REGION 1..21: note = N-terminal His tag and TEV protease cleavage site sequence
MGHHHHHHDYDIPTTENLYFQGSMKYITSKHVFFDYLNENEFVIGIGSNQEITNNKDYFNNCLNLCYFCINPKSISEILSFIKDNNIDILYFDKMKKMKFITKEIIDFNDRYSRNHLYYNALGYKIYDIQNKISKSHILIVGAGGIGNICSYLLGTIGIKKLSIIDDDIVEESNLNRQFLFREKDINKNKVETIKRELLSIRKDIIIDIFPEKLNKSILDKISQIDLVICSADDEYCIDMINEFCCFNKIPLINVGYLNDISVIGPFYIPKLEYSCCLCCDKSIYLENDVIDEKVKKIKSVTKAPSTIINNFFAGAMLGSELIKFFARDYKSMQSINSVIGIHNKNFKYEEIKLAKNYNCKYCGVNNETL

SEQ ID NO: 73 - protein, 273 aa, synthetic construct
  REGION 1..21: note = N-terminal His tag and TEV protease cleavage site sequence
  REGION 263..273: note = Linker and MccB recognition sequence
MGHHHHHHDYDIPTTENLYFQGSMVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYKGGGGMRTGNAG

SEQ ID NO: 74 - protein, 422 aa, synthetic construct
  REGION 1..21: note = N-terminal His tag and TEV protease cleavage site sequence
  REGION 412..422: note = Linker and MccB recognition sequence
MGHHHHHHDYDIPTTENLYFQGSMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRGGGGGMRTGNAG

SEQ ID NO: 75 - protein, 387 aa, synthetic construct
  REGION 377..387: note = Linker and MccB recognition sequence
MHHHHHHKEETPETPETDSEEEVTIKANLIFANGSTQTAEFKGTFEKATSEAYAYADTLKKDNGEYTVDVADKGYTLNIKFAGKEKTPEEPKEEVTIKANLIYADGKTQTAEFKGTFEEATAEAYRYADALKKDNGEYTVDVADKGYTLNIKFAGKEKTPEEPKEEVTIKANLIYADGKTQTAEFKGTFEEATAEAYRYADLLAKENGKYTVDVADKGYTLNIKFAGKEKTPEEPKEEVTIKANLIYADGKTQTAEFKGTFAEATAEAYRYADLLAKENGKYTADLEDGGYTINIRFAGKKVDEKPEEKEQVTIKENIYFEDGTVQTATFKGTFAEATAEAYRYADLLSKEHGKYTADLEDGGYTINIRFAGGGGSGGGSMRTGNAG

SEQ ID NO: 76 - protein, 355 aa, synthetic construct
  REGION 1..21: note = N-terminal His tag and TEV protease cleavage site sequence
  REGION 345..355: note = Linker and MccB recognition sequence
MGHHHHHHDYDIPTTENLYFQGSMEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHNGGGGMRTGNAG

SEQ ID NO: 77 - protein, 272 aa, synthetic construct
  REGION 1..21: note = N-terminal His tag and TEV protease cleavage site sequence
  REGION 263..272: note = Linker and eSrtA recognition sequence
MGHHHHHHDYDIPTTENLYFQGSMVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYKGGGGLPETGG

SEQ ID NO: 78 - protein, 377 aa, synthetic construct
  REGION 1..120: note = N-terminal His tag and SUMO fusion sequence
  REGION 364..377: note = sortase recognition sequence
MGHHHHHHDYDIPTTENLYFQGSSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGGGGLPETGGAAASRSGC

SEQ ID NO: 79 - protein, 510 aa, synthetic construct
  REGION 244..250: note = MccB recognition sequence
  REGION 251..510: note = Mxe GyrA intein
MVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYKGGGGMRTGNAGCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHATGLTGLNSGLTTNPGVSAWQVNTAYTAGQLVTYNGKTYKCLQPHTSLAGWEPSNVPALWQLQ

SEQ ID NO: 80 - protein, 181 aa, synthetic construct
  REGION 1..21: note = N-terminal His tag and TEV protease cleavage site sequence
MGHHHHHHDYDIPTTENLYFQGSMSGIALSRLAQERKAWRKDHPFGFVAVPTKNPDGTMNLMNWECAIPGKKGTPWEGGLFKLRMLFKDDYPSSPPKCKFEPPLFHPNVYPSGTVCLSILEEDKDWRPAITIKQILLGIQELLNEPNIQDPAQAEAYTIYCQNRVEYEKRVRAQAKKFAPS

SEQ ID NO: 81 - protein, 276 aa, synthetic construct
  REGION 1..21: note = N-terminal His tag and TEV protease cleavage site sequence
MGHHHHHHDYDIPTTENLYFQGMRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSGPRKVIKMESEEGSGSVQLADHYQQNTPIGDGPVLLPDNHYLSTQSVLSKDPNEKRDHMVLLEFVTAAGITHGMDELYK

SEQ ID NO: 82 - protein, 236 aa, synthetic construct
MKSLLPTAAAGLLLLAAQPAMASDIQMTQSPSSLSASVGDRVTITCRASQSVSSAVAWYQQKPGKAPKLLIYSASSLYSGVPSRFSGSRSGTDFTLTISSLQPEDFATYYCQQSWGLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 261 aa, synthetic construct
  REGION 250..261: note = Linker and MccB recognition sequence
MKKNIAFLLASMFVFSIATNAYAEISEVQLVESGGGLVQPGGSLRLSCAASGFNISYYSIHWVRQAPGKGLEWVASIYPYYSSTSYADSVKGRFTISADTSKNTAYLQMNSLRAEDTAVYYCARAGWVASSGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGGMRTGNAG

SEQ ID NO: 84 - protein, 390 aa, synthetic construct
  PROPEP 1..108
MRGKKVWISLLFALALIFTMAFGSTSSAQAAGKSNGEKKYIVGFKQTMSTMSAAKKKDVISEKGGKVQKQFKYVDAASATLNEKAVKELKKDPSVAYVEEDHVAHAYAQSVPYGVSQIKAPALHSQGYTGSNVKVAVIDSGIDSSHPDLKVAGGASFVPSETNPFQDNNSHGTHVAGTVAALDNSIGVLGVAPSASLYAVKVLGADGSGQYSWIISGIEWAIANNMDVINLALGGPSGSAALKAAVDKAVASGVVVVAAAGNEGTSGSSSTVGYPGKYPSVIAVGAVDSSNQRASFSSVGPELDVMAPGVSIQSTLPGNRYGAYSGTCMASAHVAGAAALILSKHPNWTNTQVRSSLENTTTKLGDSFYYGKGLINVQAAAQLEHHHHHH

SEQ ID NO: 85 - protein, 156 aa, synthetic construct
MQAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVKLEHHHHHH

SEQ ID NO: 86 - protein, 7 aa, synthetic construct
PDGFRTM

SEQ ID NO: 87 - protein, 339 aa, synthetic construct
  REGION 1..20: note = N-terminal IgK leader sequence
  REGION 21..36: note = V5 tag
  DOMAIN 291..339: note = C-terminal PDGF receptor transmembrane domain
METDTLLLWVLLLWVPGSTGGKPIPNPLLGLDSTGSGGGASMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGGSGGGGSAVGQDTQEVIVVPHSLPFKVVVISAILALVVLTIISLIILIMLWQKKPR

SEQ ID NO: 88 - protein, 7 aa, synthetic construct
  MOD_RES 7: note = Mesna thioesterification
MLGLRGG

SEQ ID NO: 89 - protein, 7 aa, synthetic construct
  MOD_RES 7: note = Mesna thioesterification
MRGLRGG

SEQ ID NO: 90 - protein, 7 aa, synthetic construct
  MOD_RES 7: note = Mesna thioesterification
MRGRRGG

SEQ ID NO: 91 - protein, 7 aa, synthetic construct
  MOD_RES 7: note = Mesna thioesterification
MRGRRLG

SEQ ID NO: 92 - protein, 11 aa, synthetic construct
PRKVIKMESEE